SEQUENCE LISTING (1 sequence)

SEQ ID NO: 1 - RNA, 101 nt, synthetic construct
  source 1..101: mol_type = other RNA
agacgcaactgaatgaaacattcccaccttagagtgactgtgcctgcttcggcaggacaggtgcatgtcaaccgctgtcactccgtaactagtcgcgtcac

(19) United States
(12) Patent Application Publication
(10) Pub. No.: US 2026/0327485 A1
(43) Pub. Date: Oct. 22, 2026

(54) MICROENVIRONMENT VISCOSITY SENSITIVE FLUORESCENT DYE, PREPARATION METHOD THEREFORE, AND USE THEREOF

(71) Applicant: SHANGHAI JIAOTONG UNIVERSITY, Shanghai (CN)

(72) Inventors: Linyong ZHU, Shanghai (CN); Xinyi HUANG, Shanghai (CN); Li JIANG, Shanghai (CN); Qiuning LIN, Shanghai (CN)

(21) Appl. No.: 19/482,342

(22) PCT Filed: May 8, 2024
(86) PCT No.: PCT/CN2024/091649

§ 371 Date: Nov. 7, 2025

(30) Foreign Application Priority Data
May 9, 2023 (CN) 202310513329.7

(51) Int. Cl. C09K 11/06; A61K 49/00; G01N 21/64

(52) U.S. Cl. CPC C09K 11/06; A61K 49/0021; G01N 21/6428; C09K 2211/1048; C09K 2211/1051; C09K 2211/1062; C09K 2211/1066; C09K 2211/1096

(57) ABSTRACT

Microenvironment viscositysensitive fluorescent dye, preparation method therefore and use thereof. The fluorescent molecules described in this invention exhibits negligible fluorescence emission in its free state but demonstrates activated fluorescence in high-viscosity microenvironments such as glycerol. It is a new microenvironmental viscosity-sensitive fluorescent dye with obvious viscosity responsiveness, whose fluorescence intensity varies with the viscosity of the microenvironment, and a high viscosity results in a strong fluorescence intensity. Furthermore, the fluorescent molecules exhibit extended emission wavelengths and enhanced brightness while maintaining superior photostability under high-intensity laser excitation, effectively resisting photobleaching. Additionally, this class of dye molecules demonstrates prolonged fluorescence lifetimes that exhibit microenvironment-dependent variations.

### TECHNICAL FIELD

The present invention relates to a fluorescent dye sensitive to microenvironmental viscosity, preparation method therefore and use thereof.

### BACKGROUND ART

Fluorescence imaging-based techniques have become an indispensable tool in biomedical research, and the development of fluorescent dyes has become an important part of chemical biology. Among them, the development of small-molecule fluorescent dyes is particularly important. Due to their small molecular structure, rapid penetration of the cell membrane, high sensitivity, and fast response time, they have more advantages than fluorescence protein-based probes in optical imaging and analytical sensing. Currently, fluorescent dyes have been widely used in cell staining, the construction of environmental sensors, and the fluorescent labeling of biomolecules. Therefore, a large number of fluorescent dyes have been developed over the past few decades. Most of these fluorescent dyes are developed based on the “parent nuclei” of classical dyes (such as coumarin, fluorescein, BODIPY or cyanine) (L. D. Lavis, ACS Chem. Biol. 2008, 3, 3, 142-155; L. D. Lavis, ACS Chem. Biol., 2014, 9, 855-866.). However, most of them are always-on fluorescent dyes, which have high background fluorescence during fluorescence imaging in living cells. In addition, most of the fluorescent dyes developed based on these classical parent nuclei have relatively short wavelengths and poor tissue penetration. Among them, although cyanine dyes have relatively long wavelengths, they have poor biological stability, so it is difficult to apply them to fluorescence imaging in vivo.

In response to the current problem of high background fluorescence of fluorescent dyes, activatable fluorescent dyes have emerged (H. Tian, Chem. Soc. Rev., 2019, 48, 683-722. W. Huang, Chem. Soc. Rev., 2022, DOI: 10.1039/d2cs00562j.). In principle, they are non-fluorescent or have weak fluorescence before binding to the target substance for detection, and their fluorescence is turned on or significantly enhanced after binding to the target substance. Currently, there are mainly two types of activatable fluorescent dyes that are widely used: always-on fluorescent dyes modified with quenching groups and environment-sensitive fluorescent dyes. Always-on fluorescent dyes modified with quenching groups refer to compounds formed by covalently binding an always-on fluorescent dye such as coumarin, fluorescein, and rhodamine, etc. with a quenching group through a connecting chain (A. Jaschke, Angew. Chem. Int. Ed. Engl., 2013, 52, 13401-13404.). Among them, the fluorescence of the dye molecules is quenched through the occurrence of the FRET or PET effect between the dye molecules and the quenching group; and when this compound binds to the target molecule to be tested, the energy transfer effect is disrupted and the fluorescence recovers. Environment-sensitive fluorescent dyes refer to fluorescent dyes whose fluorescence changes with the different microenvironments in which the dye molecules are located, and are divided into fluorescent dyes with solvatochromic effects and viscosity-sensitive fluorescent dyes (M. Ryckelynck, Int. J. Mol. Sci., 2017, 19.). Environment-sensitive dye molecules have attracted much attention because they have a smaller molecular structure, lower background fluorescence, and higher sensitivity compared to always-on fluorescent dyes modified with quenching groups.

Although various currently developed environment-sensitive fluorescent dyes, such as CCVJ, HBI, and Thioflavin T, etc. (B. Shen, J Mater Chem B, 2020, 8, 9642-9651; G.-i. Konishi, Journal of Materials Chemistry C, 2016, 4, 2731-2743.), have been developed, they have short emission wavelengths and poor photostability. It is difficult to apply them to long-term dynamic tracking at the cellular level, and even more difficult to achieve fluorescence imaging in vivo. Therefore, it is crucial to develop environment-sensitive fluorescent dyes with long emission wavelengths, good photostability, and low background fluorescence. Currently, some breakthroughs have been made in the development of long-wavelength environment-sensitive fluorescent dyes, and the longest emission wavelength can reach 700 nm (Z. Nie, J. Am. Chem. Soc., 2021, DOI: 10.1021/jacs.1c04577.). However, these fluorescent dyes have short excitation wavelengths, and it is difficult to achieve red light excitation and red light emission. This means that although these molecules can achieve long-wavelength emission, they still cannot avoid the interference of in vivo autofluorescence (background fluorescence) caused by short-wavelength excitation. In addition, these dyes have poor photostability and low fluorescence quantum yields, and are not suitable for long-term tracking of biological substances and in vivo imaging. Therefore, it is of great significance to develop environment-sensitive fluorescent dyes with both long excitation and long emission wavelengths, good photostability, and high fluorescence quantum yields.

In addition, based on the characteristic that the fluorescence lifetime of a fluorescent dye is only related to its own structure and the microenvironment it is in (such as polarity, viscosity, etc.), and is independent of the laser intensity and whether there is light quenching (K. Suhling, J. Am. Chem. Soc. 2008, 130, 21, 6672-6673), the fluorescence lifetime is a more accurate means of indicating the structure of biomolecules or the interactions between biomolecules. In principle, for environment-sensitive dye molecules, their fluorescence lifetime can directly reflect the microenvironment in which the dye is located. However, the fluorescence lifetimes of currently developed environment-sensitive fluorescent dye molecules are generally short, and are only about 1 ns even in the microenvironment with the longest fluorescence lifetime. With such a low fluorescence lifetime, it is difficult for ordinary instruments to achieve accurate detection. Therefore, if an environment-sensitive fluorescent dye with a longer fluorescence lifetime can be developed, it will provide a necessary tool for accurate imaging and detection of biological substances both in vitro and in vivo.

### SUMMARY OF THE INVENTION

The fluorescent molecules described in this invention exhibits negligible fluorescence emission in its free state but demonstrates activated fluorescence in high-viscosity microenvironments such as glycerol. It is a new microenvironmental viscosity-sensitive fluorescent dye with obvious viscosity responsiveness, whose fluorescence intensity varies with the viscosity of the microenvironment, and a high viscosity results in a strong fluorescence intensity. Furthermore, the fluorescent molecules exhibit extended emission wavelengths and enhanced brightness while maintaining superior photostability under high-intensity laser excitation, effectively resisting photobleaching. Additionally, this class of dye molecules demonstrates prolonged fluorescence lifetimes that exhibit microenvironment-dependent variations. The structure and interactions of biological substances can be studied by monitoring the changes in the fluorescence lifetime.

Specifically, the present invention provides a fluorescent dye having a structure represented by formula (I),

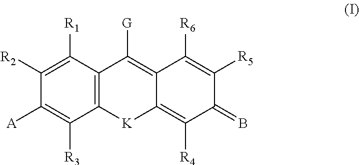

Wherein:
A is —N(X₁)(X₂) or HO—, B is ═N⁺(X₃)(X₄) or ═O, or X₁, X₂ is linked to R₂ and/or R₃, or X₃, X₄ is linked to R₄ and/or R₅ as a heterocyclic group;
X₁, X₂, X₃, and X₄ are each independently selected from the group consisting of hydrogen, alkyl, modified alkyl, aryl, heteroaryl, unsaturated or saturated heterocyclic groups, or optionally, X₁ and X₂, or X₃ and X₄ may be connected to form a saturated or unsaturated, unsubstituted or R¹⁻¹-substituted heterocyclic or heteroaromatic ring. Additionally, X₁, X₂ together with R₂ and/or R₃, or X₃, X₄ together with R₄ and/or R₅, may optionally be connected to form a saturated or unsaturated, unsubstituted or R¹⁻¹-substituted heterocyclic ring;
and/or R₁, R₂, R₃, R₄, R₅, and R₆ may optionally be independently selected from the group consisting of hydrogen, halogen atoms, nitro groups, or alkyl groups.

When K is selected from S, Se, Te, Te═O, unsubstituted or singly or doubly R¹⁻¹-substituted carbon, unsubstituted or singly or doubly R¹⁻¹-substituted Ge,

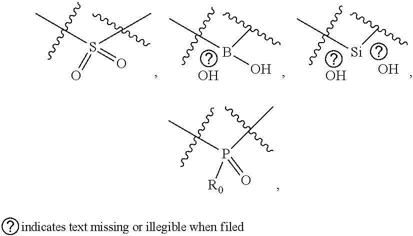

(wherein R₀ is independently and optionally selected from

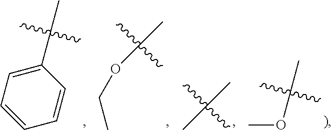

G is selected from the group consisting of formula (I-1-1), formula (I-1-2), formula (I-1-3), or formula (I-1-4).

When K is selected from O or unsubstituted or singly or doubly R¹⁻¹-substituted silicon, and A is not —N(X₁)(X₂), G is selected from the group consisting of formula (I-1-1), formula (I-1-2), formula (I-1-3), or formula (I-1-4).

When K is selected from O or unsubstituted or singly or doubly R¹⁻¹-substituted silicon, A is —N(X₁)(X₂), and B is ═N⁺(X₃)(X₄), G is selected from the group consisting of formula (I-1-2) or formula (I-1-3).

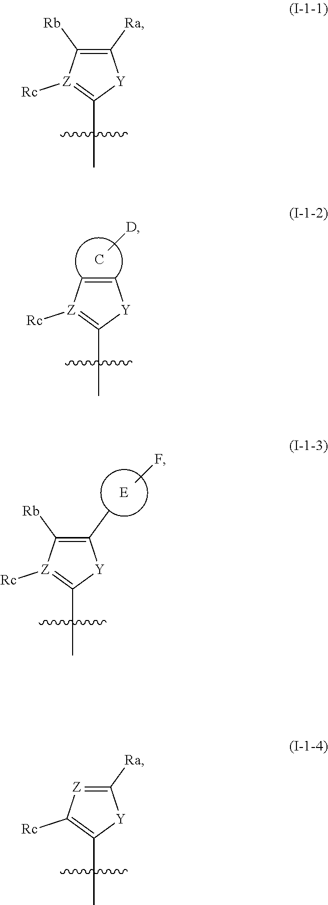

Wherein:
Each R¹⁻¹ is independently selected from the group consisting of halogen atoms or alkyl groups;
Each Y is independently selected from the group consisting of O, S, NH, Se, or Te;
Each Z is independently selected from the group consisting of C or N;
Each Rₐ, R_{b}, and R_{c} is independently selected from the group consisting of hydrogen, sulfonic acid groups, haloacyl groups, cyano groups, cyanate esters, isothiocyanates, carboxyl groups, ester groups, amide groups, nitro groups, halogen atoms, alkyl groups, haloalkyl groups, or modified alkyl groups.

The C ring of formula (I-1-2), the E ring of formula (I-1-3) are each independently selected from at least one of an aromatic ring or an aromatic heterocycle;
Each hydrogen atom in formula (I-1-2) and in formula (I-1-3) is optionally independently substituted with an atom selected from a halogen atom, or an alkyl group;
D in formula (I-1-2), F in formula (I-1-3) are each independently selected from hydrogen, cyano, amino, isothiocyano, carboxy, sulfone, phosphite, ester, amide, nitro, halogen atom, alkyl, aryl, heteroaryl, or modified alkyl;
Wherein:

The term “alkyl” refers to a C₁-C₁₀ straight-chain or branched-chain alkyl group; preferably, it is a C₁-C₇ straight-chain or branched-chain alkyl group; more preferably, it is a C₁-C₅ straight-chain or branched-chain alkyl group; most preferably, it is selected from the group consisting of methyl, ethyl, n-propyl, isopropyl, n-butyl, isobutyl, tert-butyl, sec-butyl, n-pentyl, 1-methylbutyl, 2-methylbutyl, 3-methylbutyl, isopentyl, 1-ethylpropyl, neopentyl, n-hexyl, 1-methylpentyl, 2-methylpentyl, 3-methylpentyl, isohexyl, 1,1-dimethylbutyl, 2,2-dimethylbutyl, 3,3-dimethylbutyl, 1,2-dimethylbutyl, 1,3-dimethylbutyl, 2,3-dimethylbutyl, 2-ethylbutyl, n-heptyl, 2-methylhexyl, 3-methylhexyl, 2,2-dimethylpentyl, 3,3-dimethylpentyl, 2,3-dimethylpentyl, 2,4-dimethylpentyl, 3-ethylpentyl, or 2,2,3-trimethylbutyl.

The term “modified alkyl” refers to an alkyl group in which any carbon atom is substituted by at least one group selected from the group consisting of halogen atoms, —O—, —OH, —CO—, —NO₂, —CN, —S—, —SO₂—, —(S═O)—, optionally halogen-substituted aryl groups, amino groups, secondary amino groups, tertiary amino groups, heterocyclic groups, and saturated or unsaturated monocyclic or heterocyclic groups. The modified alkyl group contains 1 to 30 carbon atoms (preferably 1 to 20 carbon atoms, more preferably up to 10 carbon atoms), and any carbon-carbon single bond therein may optionally and independently be replaced by a carbon-carbon double bond or a carbon-carbon triple bond.

The substitution of the aforementioned carbon atom means that the carbon atom or the carbon atom together with the hydrogen atoms bonded to it is replaced by the corresponding group.

The term “heterocyclic group” refers to a 4-10 membered monocyclic or polycyclic heterocyclic group containing at least one heteroatom selected from N, O, S, or Si in the ring.

The term “aryl” or “aromatic ring” refers to a 5-10 membered monocyclic or fused bicyclic aromatic group.

The term “heteroaryl” or “aromatic heterocycle” refers to a 5-10 membered monocyclic or fused bicyclic heteroaromatic group containing at least one heteroatom selected from N, O, S, or Si in the ring.

The halogen atoms in the so-called “halogen atom”, “haloalkyl group”, “haloacyl group” and “aryl group substituted by halogen atoms” are each independently selected from F, Cl, Br and I.
The term “ester group” refers to the —(C═O)OR¹⁻² group;
The term “amide group” refers to the —CONR¹⁻² group;
The term “sulfonyl group” refers to the —SO₂R¹⁻² group;
The term “sulfoxide group” refers to the —SOR¹⁻² group;
The term “secondary amino group” refers to the —NHR¹⁻² group;
The term “tertiary amino group” refers to the —NR¹⁻²R¹⁻³ group;
Each of R¹⁻² and R¹⁻³ is independently selected from H, alkyl, modified alkyl, or a halogen atom.

Optionally, in the above-mentioned fluorescent dye,
X₁ and X₂ may optionally be connected to R₂ and/or R₃ to form a saturated or unsaturated heterocyclic group, which is selected from any of the following groups:

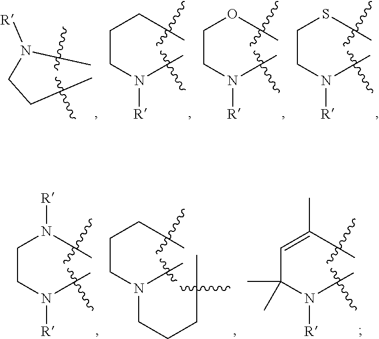

X₃ and X₄ may optionally be connected to R₄ and/or R₅ to form a saturated or unsaturated heterocyclic group, which is selected from any of the following groups:

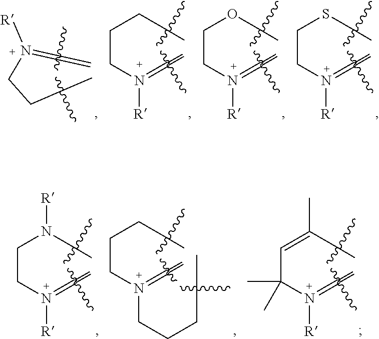

Preferably, the heterocyclic group formed by X₁, X₂, and R₂ and/or R₃, or by X₃, X₄, and R₄ and/or R₅, has a symmetrical structure;

Preferably, each R′ is independently selected from hydrogen or an alkyl group;

Preferably, X₁ and X₂ may optionally interconnect to form a saturated or unsaturated heterocyclic group selected from any of the groups represented by the following formulas, wherein the groups may optionally be substituted with a halogen atom;

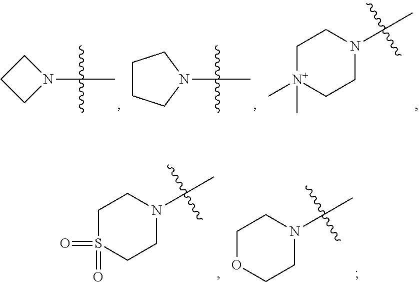

Preferably, X₃ and X₄ may optionally interconnect to form a saturated or unsaturated heterocyclic group selected from any of the groups represented by the following formulas, wherein the groups may optionally be substituted with a halogen atom;

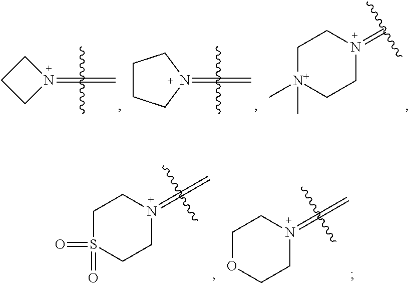

Preferably, the heterocyclic groups formed by X₁ and X₂, and by X₃ and X₄, have a symmetrical structure;

Preferably, K is selected from oxygen, sulfur, selenium, tellurium, or the following groups:

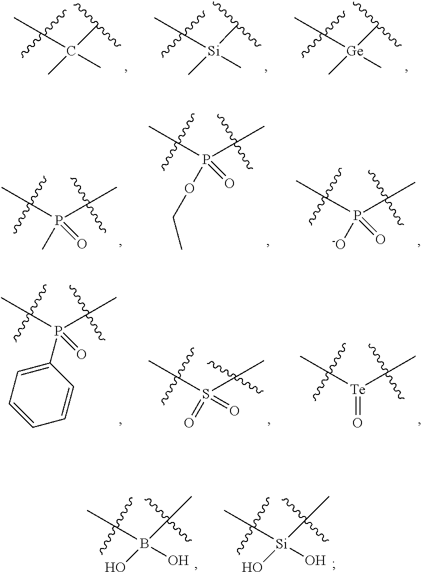

Optionally, in the above-mentioned fluorescent dye,

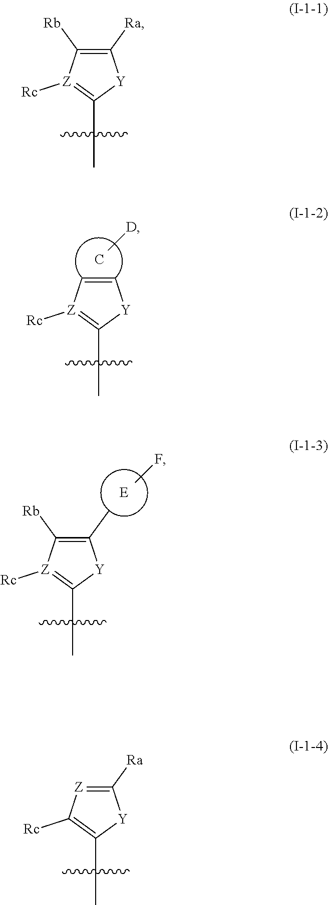

Preferably, the groups represented by formulae (I-1-1) and (I-1-4) are selected from any of the following groups:

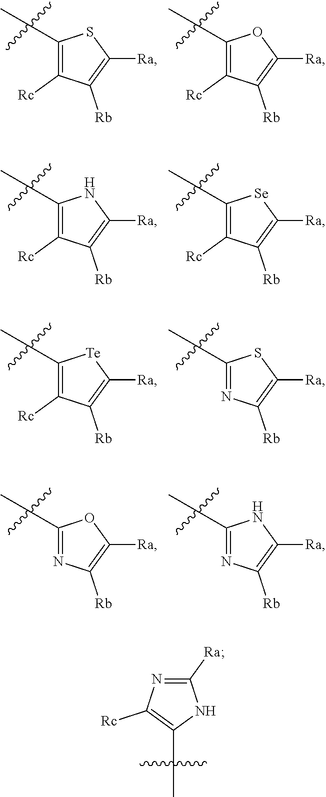

Preferably, the group represented by formula (I-1-2) can be selected from any of the following groups:

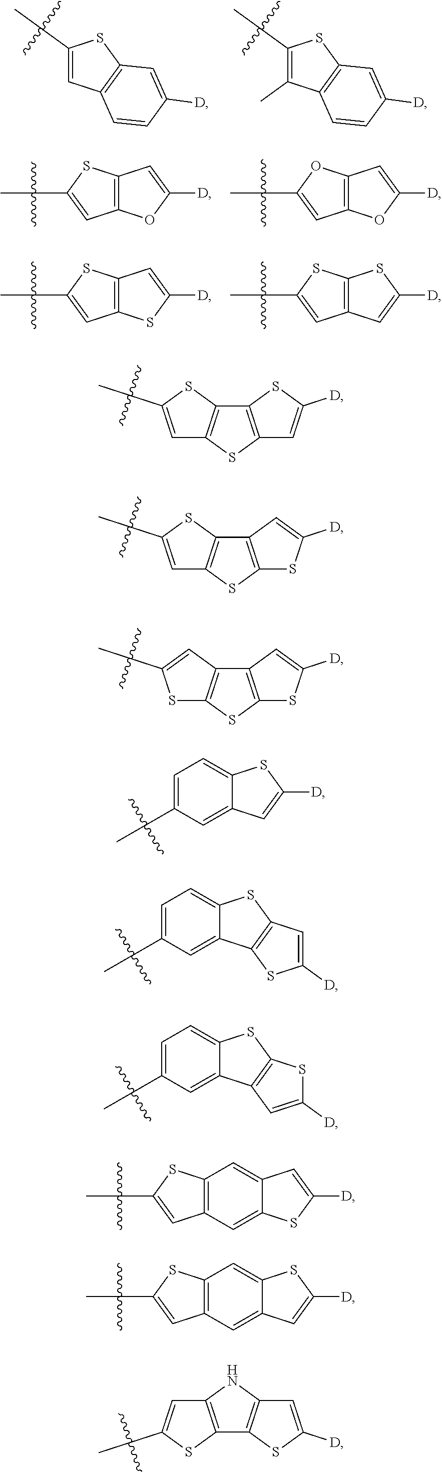

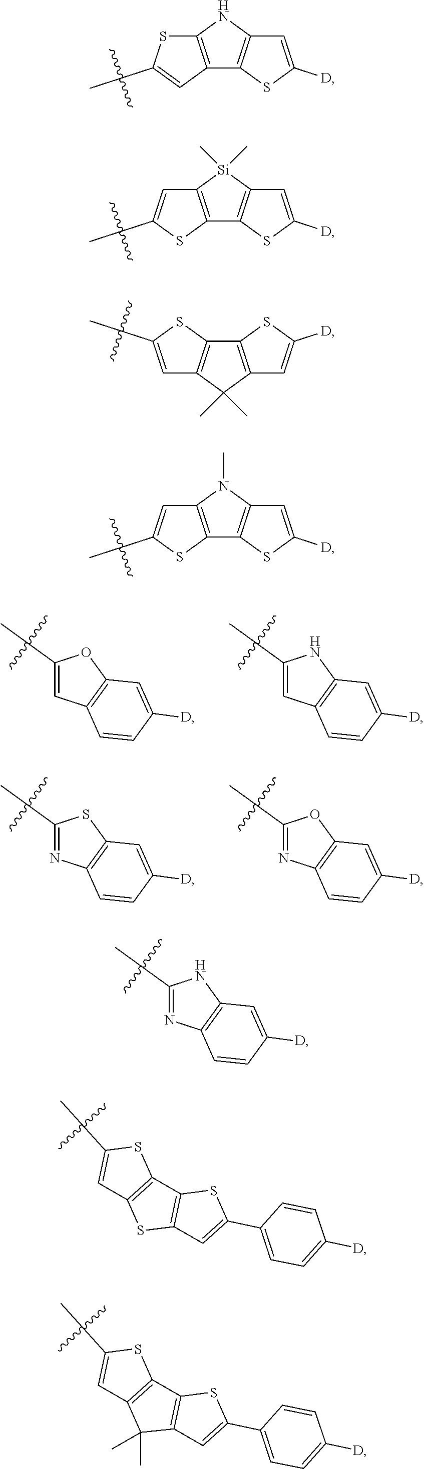

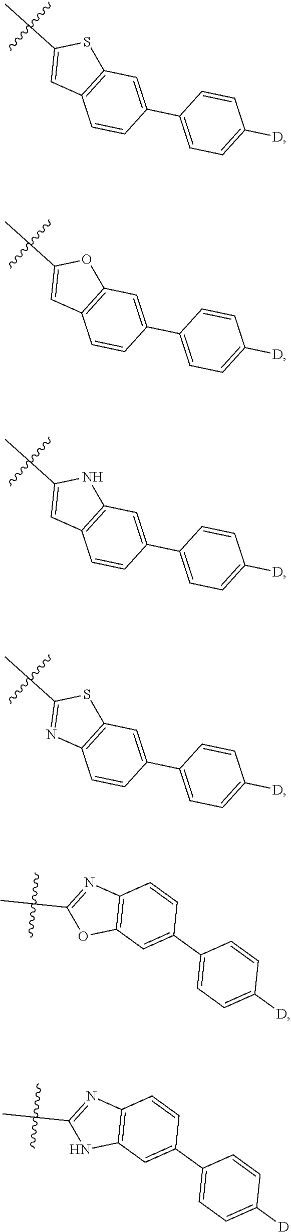

Optionally, the group represented by formula (I-1-3) can be selected from any of the following groups:

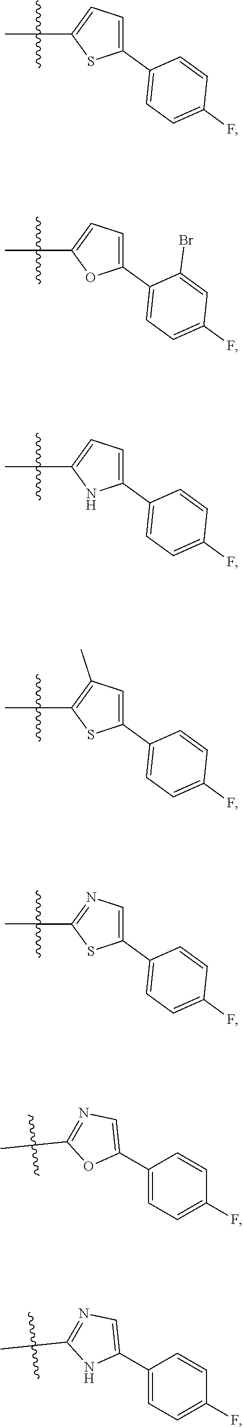

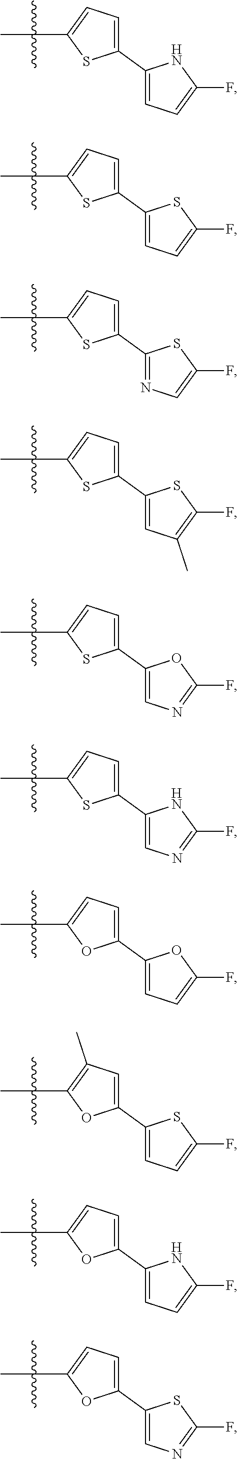

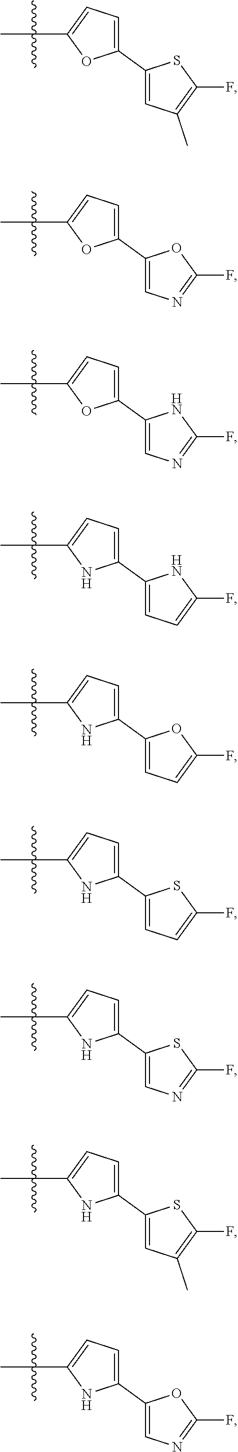

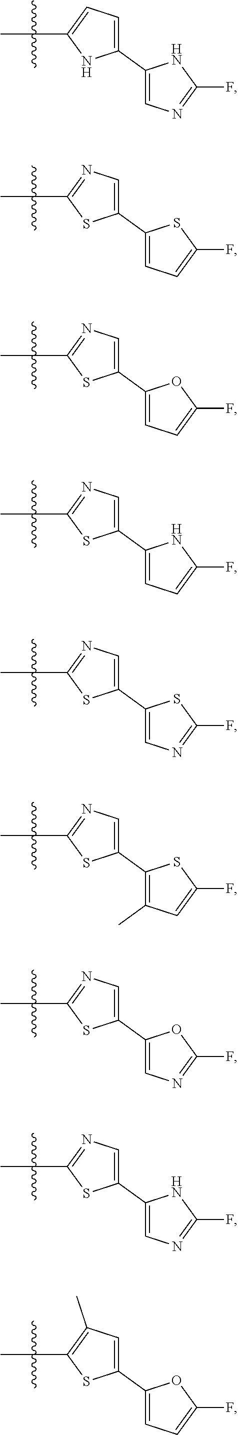

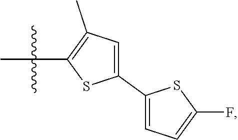 [SMILES: *c1sc(-c2ccc(F)s2)cc1C]

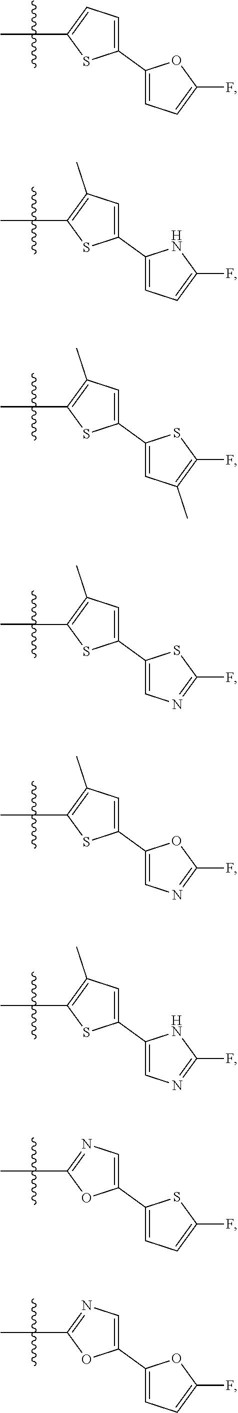

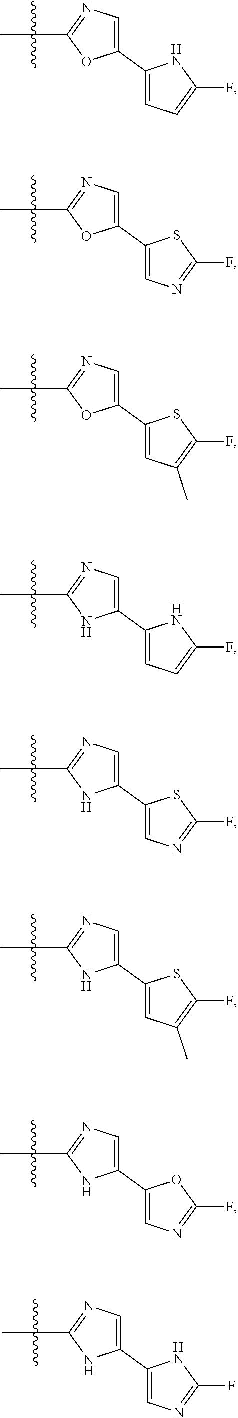

Optionally, the fluorescent dye represented by formula (I) of the present invention is selected from the following compounds:

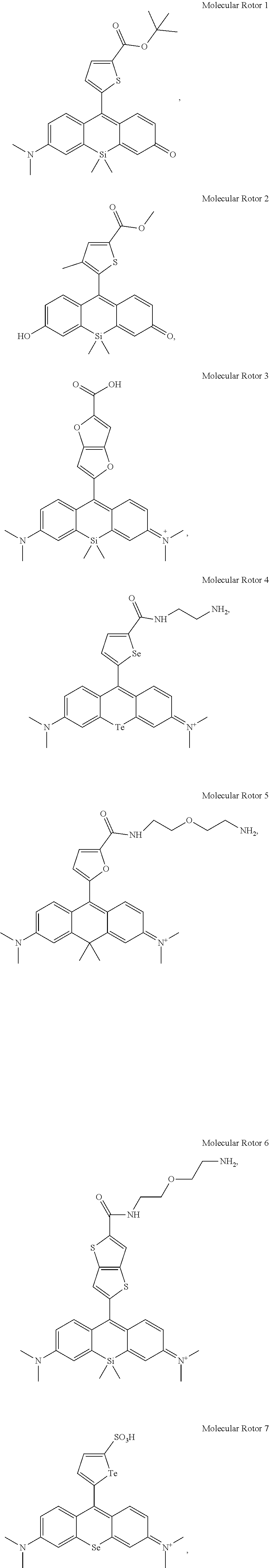

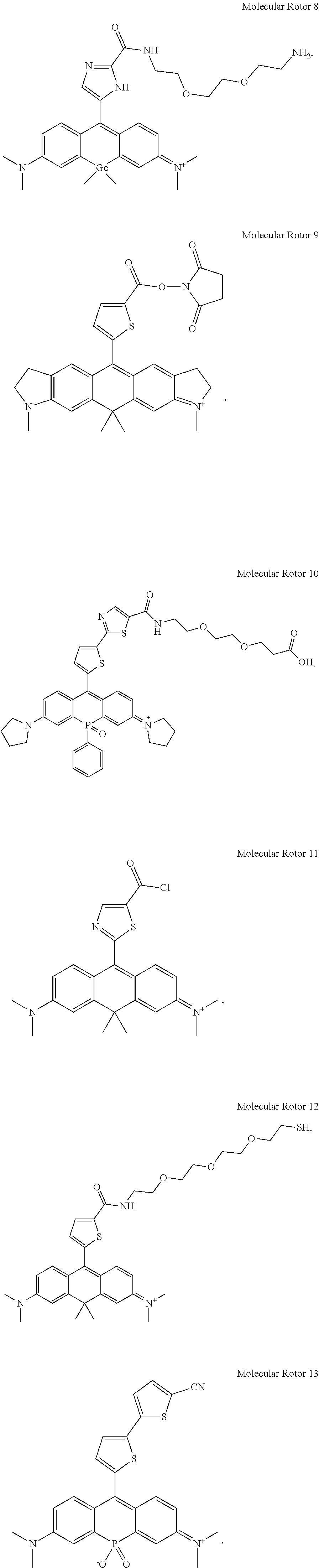

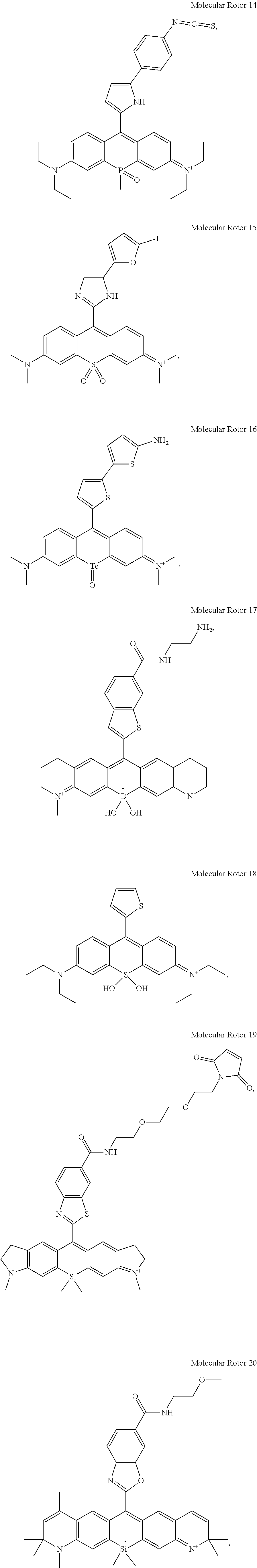

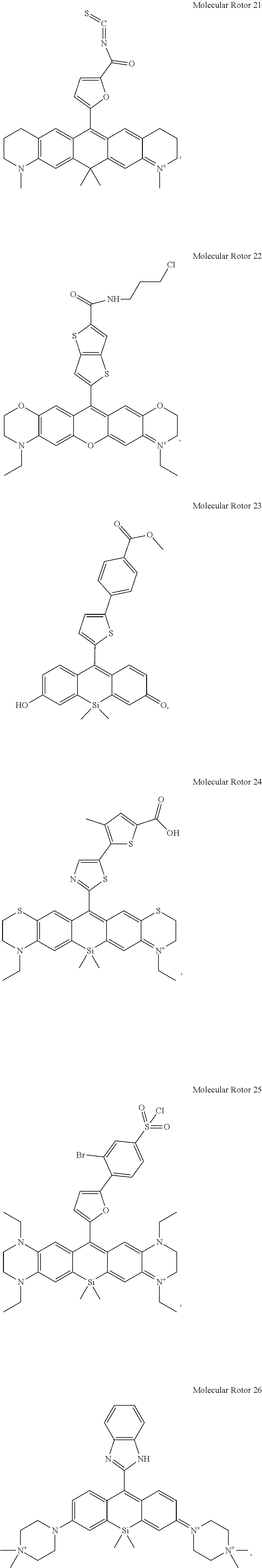

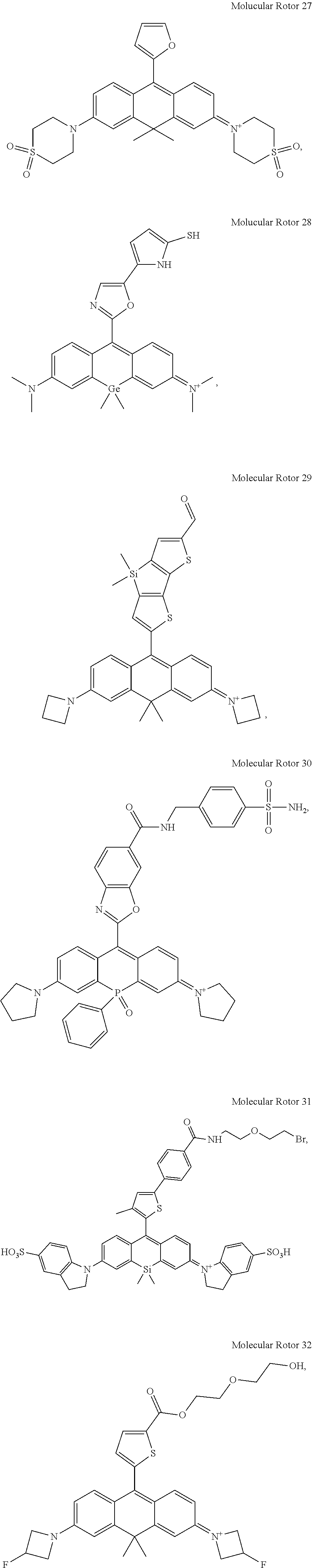

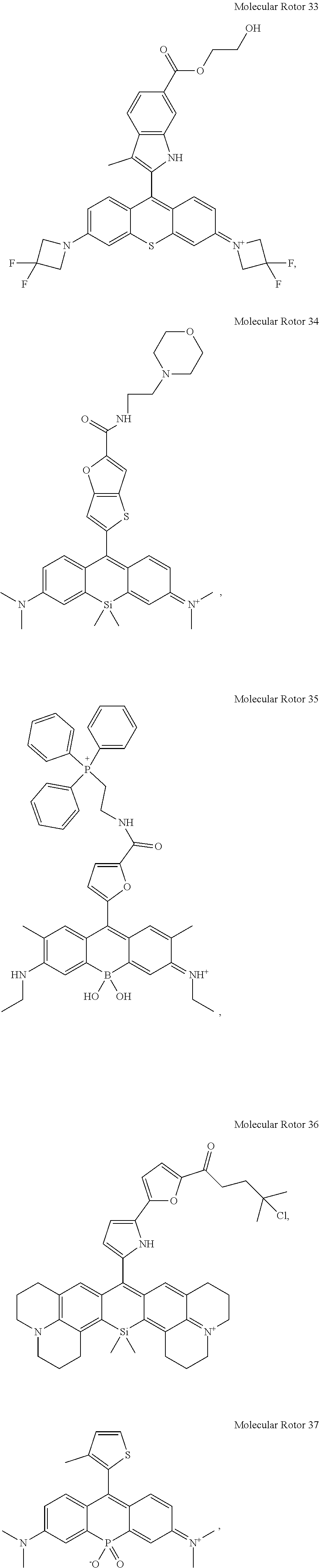

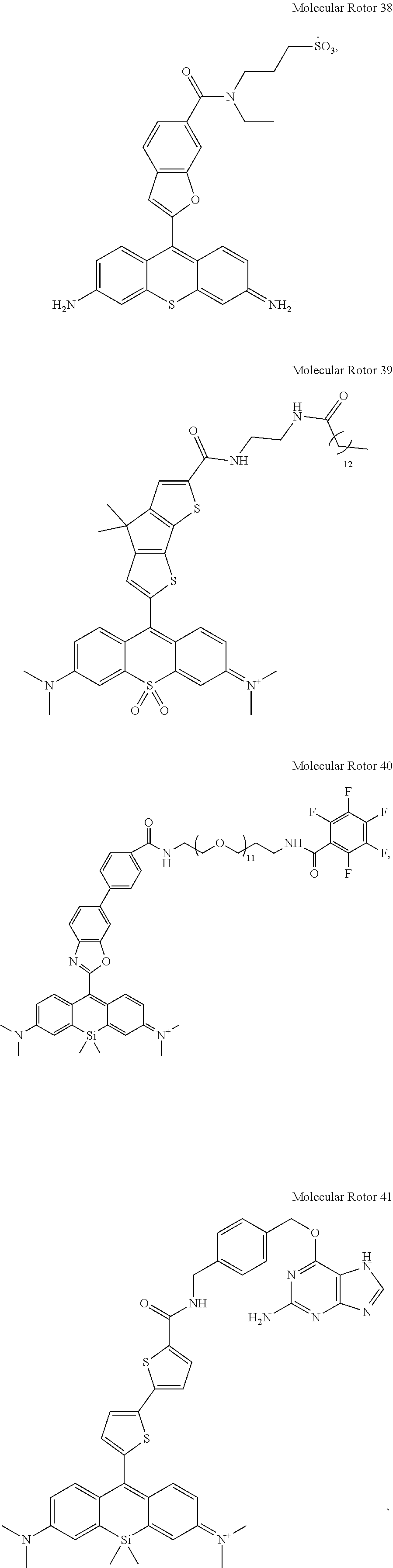

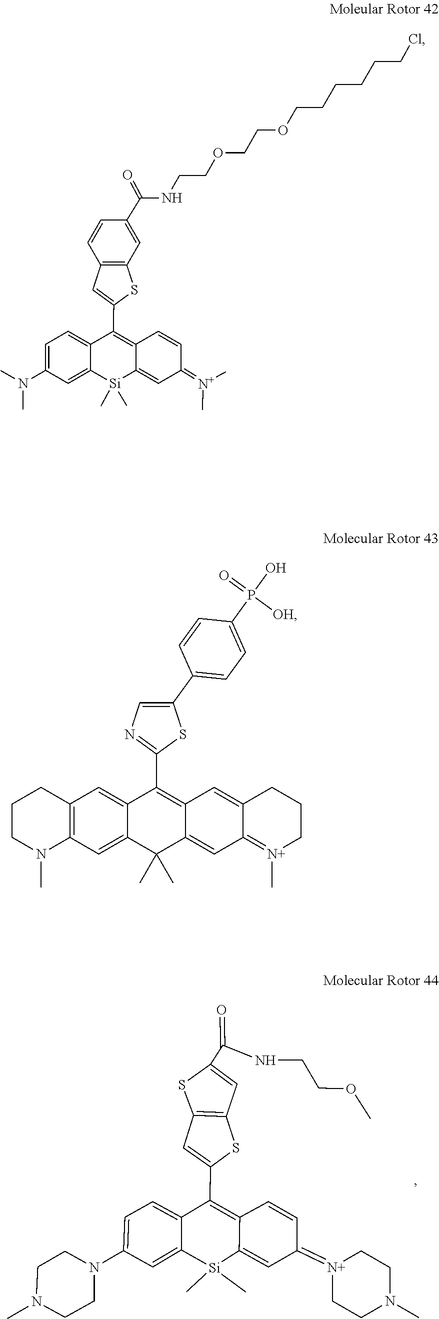

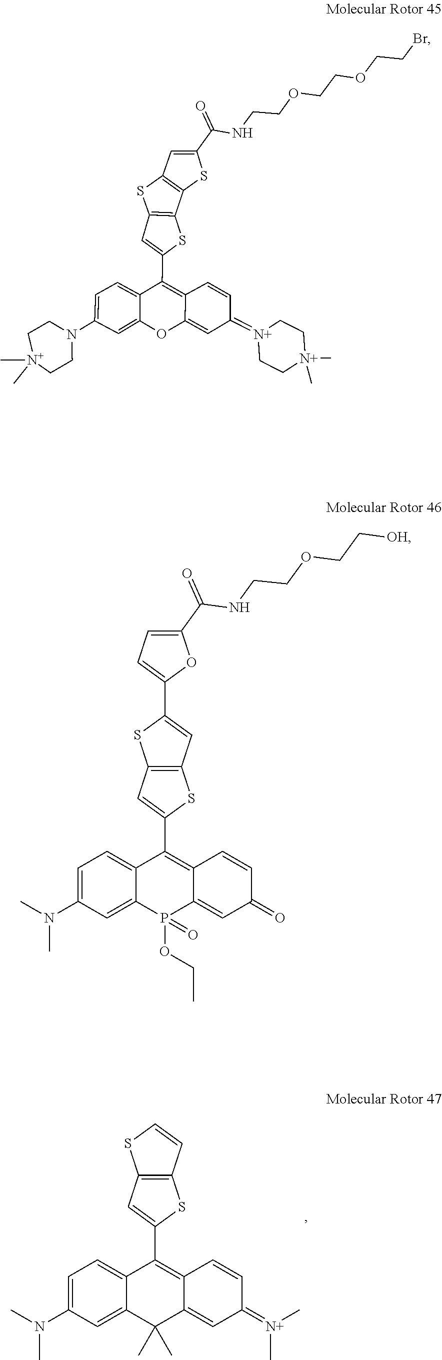

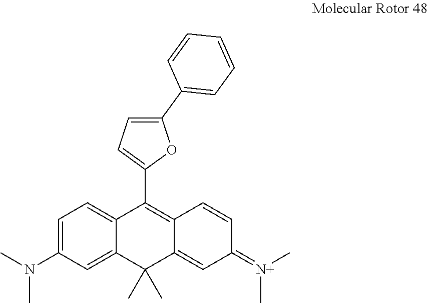

The viscosity responsiveness of the above-mentioned fluorescent dye means that the fluorescence intensity of the dye molecules responds to the viscosity of the solution, and the fluorescence intensity increases as the viscosity of the solution increases. Optionally, the fluorescent dye with viscosity responsiveness refers to an organic dye molecule for which, under the conditions of the same concentration and excitation wavelength at 25° C., the ratio of the maximum fluorescence emission intensity of the dye in glycerol to that in methanol is greater than 10, preferably greater than 100, and more preferably greater than 1000. The concentration range of the viscosity-responsive dye is between 1×10⁻⁷ M-1×10⁻⁵ M.

The structure of the above-mentioned fluorescent dye is adjustable, and it has a long fluorescence emission wavelength, which can reach up to 900 nm at the longest. According to the needs, the operator can select dyes of different types or with different emission/excitation wavelengths.

The fluorescence intensity of the above-mentioned fluorescent dye increases with the increase of the environmental viscosity. It is sensitive to the viscosity response and has viscosity responsiveness.

The above-mentioned fluorescent dye has excellent photostability. It is not prone to photobleaching under laser irradiation and can be used for long-term dynamic tracking of target molecules.

The above-mentioned fluorescent dye can be used for the specific labeling of target substances such as proteins or nucleic acids. The fluorescence of the fluorescent dye can be activated after it binds to the target substance. It has good fluorescent molecular switch properties, and the fluorescence activation multiple is high, and the fluorescence activation brightness is also high.

The above-mentioned fluorescent dye labels the substance to be tested very quickly.

There is a good linear relationship between the fluorescence intensity of the above-mentioned fluorescent dye and the concentration of the substance to be tested, which can be used for the quantitative detection of the substance to be tested. The substances to be tested include but are not limited to proteins, nucleic acids, metabolic molecules, etc.

The above-mentioned fluorescent dye can achieve specific labeling of biological substances inside cells and realize specific fluorescence lighting. At the same time, the fluorescence of the probe is not affected by the intracellular environment.

The above-mentioned fluorescent dye can serve as a powerful tool for the labeling of subcellular organelles, such as the labeling of the nucleus, mitochondria, Golgi apparatus, endoplasmic reticulum, whole cells, cytoskeleton, extracellular membrane, lysosome, intracellular membrane, etc.

The spectra of the fluorescent groups of the above-mentioned fluorescent dyes will not interfere with each other. The fluorescent dyes of different color systems of the present invention can perform multi-color labeling on samples and can simultaneously perform orthogonal labeling imaging.

The above-mentioned fluorescent dye has a relatively long fluorescence lifetime, and the fluorescence lifetime changes with the change of the microenvironment in which the dye is located.

In the second aspect of the present invention, a preparation method of the fluorescent dye represented by formula (I) as described above is provided. The method comprises reacting the brominated aromatic ring compound represented by formula (II-1-1), or formula (II-1-2), or formula (II-1-3), or formula (II-1-4) with the ketone represented by (II-2) to obtain the fluorescent dye represented by formula (I):

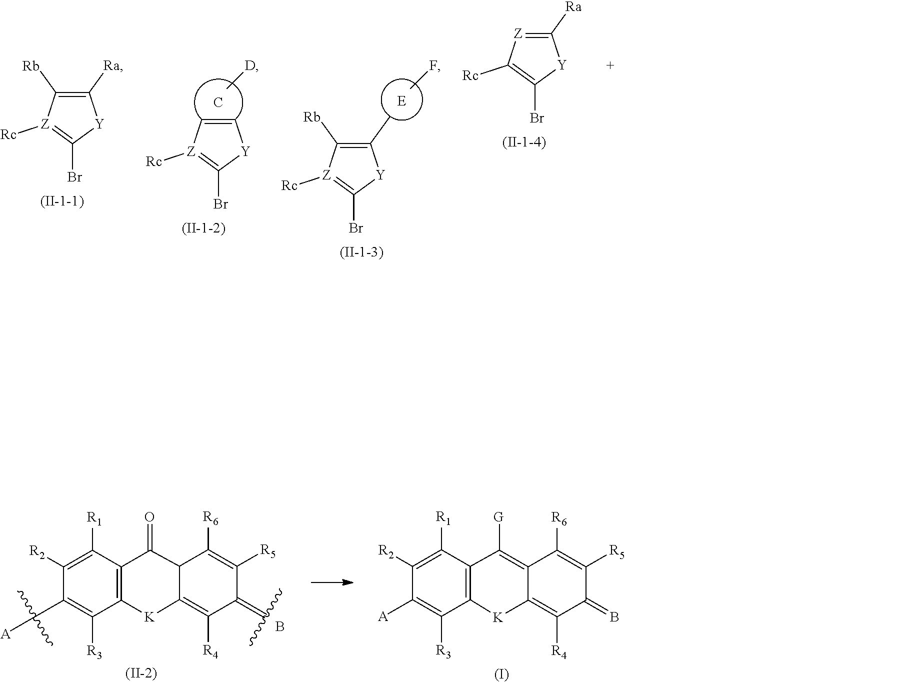

In the third aspect of the present invention, a method for specifically labeling a biological substance with fluorescence activation is provided, which comprises the following steps: After contacting the above-mentioned fluorescent dye with abiological substance (including but not limited to proteins, RNA or DNA), the dye undergoes covalent or non-covalent binding with the biological substance, thereby specifically activating the fluorescence of the dye molecules and achieving specific fluorescent labeling of the biological substance. Those skilled in the art can use the instrument equipment with corresponding configuration to track and monitor the target substance labeled with the dye. According to the needs, the used instrument equipment includes the equipment and facilities capable of testing or displaying fluorescence, such as a fluorescence spectrometer, a fluorescence microscope, a confocal fluorescence microscope, a microplate reader, a flow cytometer and other equipment.

Optionally, the reaction medium for the labeling reaction is selected from a pure solution of the substance to be tested (such as pure proteins, pure DNA and RNA, etc.), a cell lysate or the in-situ medium in which the substance to be tested is located; optionally, the in-situ medium is an intracellular medium, an organelle medium, a living tissue medium, blood or a body fluid.

In the fourth aspect of the present invention, a probe kit is provided, which is characterized by comprising the above-mentioned fluorescent dye.

Optionally, the probe kit further contains a biocompatible medium; optionally, the biocompatible medium is selected from at least one of dimethyl sulfoxide, a buffer agent, and physiological saline; optionally, the buffer agent includes phosphate buffered saline.

In the fifth aspect of the present invention, a pharmaceutical preparation is provided, which is characterized by comprising the above-mentioned fluorescent dye.

Optionally, the pharmaceutical preparation is a pharmaceutical composition made by mixing the fluorescent dye with other compounds, which is suitable for administration to animals or humans. The pharmaceutical composition further includes a physiologically acceptable carrier for animals or humans.

Optionally, the physiologically acceptable carrier includes water and saline, and may also include pharmaceutical reagents such as buffers, as well as other pharmaceutical reagents, such as compatible preservatives used in pharmaceutical dosage forms. Preferably, the carrier is a liquid, and more preferably an aqueous solution.

In the sixth aspect of the present invention, the in vitro application of the above-mentioned fluorescent dye is also provided. The in vitro application includes the use of the fluorescent dye in the preparation of pharmaceutical preparations for in vitro disease detection, including but not limited to the way of labeling the fluorescent dye molecules on antibodies and detecting various diseases through antigen-antibody interactions; or using the fluorescent dye molecules as targets for the screening of aptamers, and using the screened aptamers for the fluorescent imaging of target nucleic acids and as probes for the detection of various disease markers.

In the seventh aspect of the present invention, the in vivo application of the above-mentioned fluorescent dye is also provided. The in vivo application includes the use of the above-mentioned fluorescent dye in the preparation of an optical imaging system for in vivo fluorescence imaging detection or treatment; optionally, the in vivo fluorescence imaging detection or treatment includes performing fluorescence-guided surgery or ex vivo tissue staining on humans or animals through fluorescence imaging or fluorescence lifetime imaging.

The fluorescence imaging carried out in animal experimental studies includes but is not limited to in vivo imaging, brain imaging, etc. The fluorescence imaging carried out on humans includes but is not limited to fluorescence-guided surgery and ex vivo tissue staining.

The above-mentioned in vivo application should include a complete set of optical imaging systems that can support in vivo fluorescence imaging, including but not limited to an in vivo imaging instrument and various optical fibers, cavity mirrors, endoscopes, etc. used in conjunction with human fluorescence imaging.

In the eighth aspect of the present invention, a method for in vitro disease detection is provided, including the way of labeling fluorescent dye molecules on antibodies and detecting various diseases through antigen-antibody interactions; or using the fluorescent dye molecules as targets for the screening of aptamers, and using the screened aptamers for the fluorescent imaging of target nucleic acids and as probes for the detection of various disease markers.

In the ninth aspect of the present invention, a method for in vivo fluorescence imaging detection or treatment is provided, including performing fluorescence-guided surgery or ex vivo tissue staining on humans or animals through fluorescence imaging or fluorescence lifetime imaging;

Optionally, the optical imaging system includes an imaging instrument or various optical fibers, cavity mirrors, or endoscopes used in conjunction with human or animal fluorescence imaging.

### DESCRIPTION OF THE DRAWINGS

FIG. 1 shows the fluorescence emission intensity diagram of molecular rotor 2 (1×10⁻⁵ M) under different viscosity conditions;

FIG. 2 shows the linear relationship diagram between the viscosity conditions and the fluorescence intensity of molecular rotor 2 (1×10⁻⁵ M);

FIG. 3 shows the fluorescence emission intensity diagram of molecular rotor 9 (1×10⁻⁵ M) under different viscosity conditions;

FIG. 4 shows the linear relationship diagram between the viscosity conditions and the fluorescence intensity of molecular rotor 9 (1×10⁻⁵ M);

FIG. 5 shows the fluorescence emission intensity diagram of molecular rotor 12 (1×10⁻⁵ M) under different viscosity conditions;

FIG. 6 shows the linear relationship diagram between the viscosity conditions and the fluorescence intensity of molecular rotor 12 (1×10⁻⁵ M);

FIG. 7 shows the fluorescence emission intensity diagram of molecular rotor 19 (1×10⁻⁵ M) under different viscosity conditions;

FIG. 8 shows the linear relationship diagram between the viscosity conditions and the fluorescence intensity of molecular rotor 19 (1×10⁻⁵ M);

FIG. 9 shows the fluorescence emission intensity diagram of molecular rotor 40 (1×10⁻⁵ M) under different viscosity conditions;

FIG. 10 shows the linear relationship diagram between the viscosity conditions and the fluorescence intensity of molecular rotor 40 (1×10⁻⁵ M);

FIG. 11 shows the photostability test of molecular rotors 6, 12, 20, 40, 42 and comparative molecules 1, 2;

FIG. 12 shows the fluorescence lifetime diagram of molecular rotor 6 (1×10⁻⁵ M) under different viscosity conditions;

FIG. 13 shows the fluorescence lifetime diagram of molecular rotor 12 (1×10⁻⁵ M) under different viscosity conditions;

FIG. 14 shows the fluorescence lifetime diagram of molecular rotor 19 (1×10⁻⁵ M) under different viscosity conditions;

FIG. 15 shows the fluorescence lifetime diagram of molecular rotor 20 (1×10⁻⁵ M) under different viscosity conditions;

FIG. 16 shows the fluorescence lifetime diagram of molecular rotor 41 (1×10⁻⁵ M) under different viscosity conditions;

FIG. 17 shows the fluorescence lifetime diagram of comparative molecule 3 (1×10⁻⁵ M) under different viscosity conditions;

FIG. 18 shows the fluorescence lifetime diagram of comparative molecule 4 (1×10⁻⁵ M) under different viscosity conditions;

FIG. 19 shows the comparison diagram of the fluorescence background in PBS of molecular rotors 6, 12, 20, 40, 42 and comparative molecules 1, 2 (1×10⁻⁶ M).

FIG. 20 shows the diagram of the specific localization of molecular rotors 34, 35, 39 and 40 in subcellular organelles (such as lysosomes, mitochondria, cell membranes, endoplasmic reticulum, etc.);

FIG. 21 shows the fluorescence imaging diagram of the target proteins in living cells after the specific binding of molecular rotors 22, 41 and 42 with the protein tags Halo-Tag or SNAP-Tag.

FIG. 22 shows the fluorescence imaging diagram of low-abundance RNA by the specific binding of molecular rotors 5, 6, 12 and 19 with RNA aptamers.

FIG. 23 shows the fluorescence imaging diagram of tumors in vivo using molecular rotors 20, 36 and 43.

### DETAILED EMBODIMENTS

The following provides a detailed description of the specific embodiments of the present invention. It should be understood that the specific embodiments described herein are only for exemplary illustration of the present invention and are not intended to limit the present invention.

### Example 1

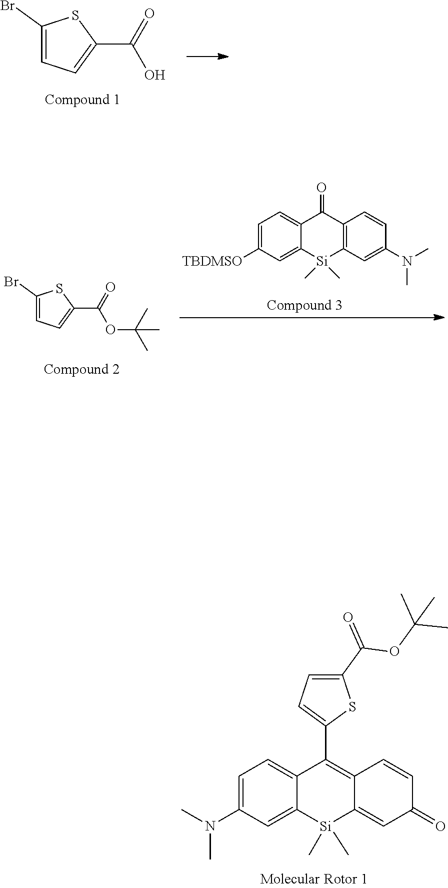 [SMILES: O=C(O)c1ccc(Br)s1 ; CC(C)(C)OC(=O)c1ccc(Br)s1 ; CN(C)c1ccc2c(c1)[Si](C)(C)c1cc(O[Si](C)(C)C(C)(C)C)ccc1C2=O ; CN(C)c1ccc2c(c1)[Si](C)(C)C1=CC(=O)C=CC1=C2c1ccc(C(=O)OC(C)(C)C)s1]

Synthesis of Compound 2: Dissolve Compound 1 (2.0 g, 9.6 mmol) in 80 mL of tert-butanol solution, add 4-dimethylaminopyridine (DMAP) (1.2 g, 9.6 mmol). After stirring the reaction system at room temperature for 10 minutes, add di-tert-butyl dicarbonate (6.3 g, 29 mmol) to the above system, and then continue to stir the reaction system at room temperature for 2 hours. After the reaction is completed, remove the solvent by filtration under reduced pressure, and purify the residue by column chromatography to obtain Compound 2 (2.1 g, yield 85%). ¹H-NMR (400 MHz, DMSO-d₆): δ=7.99 (d, J=1.6 Hz, 1H), 7.65 (d, J=1.6 Hz, 1H), 1.51 (s, 9H).

Compound 3 was synthesized by referring to the literature Angew. Chem. Int. Ed. 2018,57,15702-15706. ¹H-NMR (400 MHz, CDCl₃): δ=8.40 (d, J=3.7 Hz, 1H), 8.37 (d, J=3.7 Hz, 1H), 7.04 (d, J=2.9 Hz, 1H), 6.98 (dd, J=8.8 Hz, 2.2 Hz, 1H), 6.84 (dd, J=9.5 H, 2.9 Hz, 1H), 6.78 (d, J=2.9 Hz, 1H), 3.10 (s, 6H), 1.01 (s, 9H), 0.46 (s, 6H), 0.26 (s, 6H).

Synthesis of Molecular Rotor 1: In a 100 mL three-necked flask, dissolve Compound 2 (0.2 g, 0.76 mmol) in THF (50 mL), and cool the system to −78° C. under nitrogen protection. After 20 minutes, add 1.6 M n-butyllithium (0.47 mL, 0.76 mmol) dropwise to the reaction system, and keep the mixture stirring at −78° C. for another 30 minutes. Then, dissolve Compound 3 (31 mg, 0.0075 mmol) in THF and add it dropwise to the reaction system. After the dropwise addition is completed, gradually restore the reaction system to room temperature and keep stirring at room temperature for another 2 hours. After the reaction is finished, add 2 M HCl solution to quench the reaction. Extract with DCM, combine the organic phases, dry over anhydrous sodium sulfate, remove the solvent by distillation under reduced pressure, and purify the residue by column chromatography to obtain the product (23 mg, yield 66%). ¹H-NMR (400 MHz, CDCl₃): δ=7.9 (d, J=7.4 Hz, 1H), 7.34-7.36 (m, 2H), 7.12 (d, J=7.6 Hz, 1H), 7.09 (d, J=7.6 Hz, 1H), 6.78 (d, J=9.3 Hz, 1H), 6.58 (d, J=2.4 Hz, 1H), 6.33 (d, J=2.9 Hz, 1H), 3.09 (s, 6H), 1.56 (s, 9H), 0.48 (s, 6H).

### Example 2

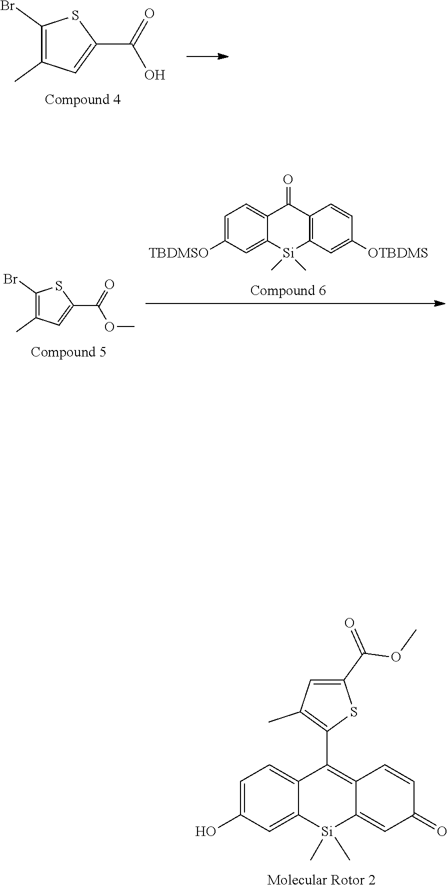

Synthesis of Compound 5: Dissolve Compound 4 (2.0 g, 9.1 mmol) in 50 mL of methanol solution, add concentrated sulfuric acid (1.5 mL) dropwise, and stir under reflux with heating overnight. The next day, remove the solvent by distillation under reduced pressure, dilute the obtained solid with water (20 mL), basify it with a saturated sodium carbonate solution, extract with ethyl acetate (EA), combine the organic phases, dry over anhydrous sodium sulfate, remove the solvent by distillation under reduced pressure, and purify by column chromatography to obtain the product (2.1 g, yield 82%). ¹H-NMR (400 MHz, CDCl₃): δ=7.54 (s, 1H), 3.87 (s, 3H), 2.57 (s, 3H).

Compound 6 was synthesized by referring to the literature Chem. Commun., 2011,47, 4162-4164. ¹H-NMR (400 MHz, CDCl₃): δ=8.37 (d, J=8.8 Hz, 2H), 7.04 (d, J=2.9 Hz, 2H), 6.98 (dd, J=2.2, 8.8 Hz, 2H), 1.01 (s, 18H), 0.46 (s, 6H), 0.26 (s, 12H).

Synthesis of Molecular Rotor 2: In a 100 mL three-necked flask, dissolve Compound 5 (0.2 g, 0.72 mmol) in THF (50 mL), and cool the system to −78° C. under nitrogen protection. After 20 minutes, add 1.6 M n-butyllithium (0.45 mL, 0.72 mmol) dropwise to the reaction system, and keep the mixture stirring at −78° C. for another 30 minutes. Then, dissolve Compound 6 (36 mg, 0.0072 mmol) in THF and add it dropwise to the reaction system. After the dropwise addition is completed, gradually restore the reaction system to room temperature and keep stirring at room temperature for another 2 hours. After the reaction is finished, add 2 M HCl solution to quench the reaction. Extract with DCM, combine the organic phases, dry over anhydrous sodium sulfate, remove the solvent under reduced pressure, and purify by column chromatography to obtain the product (23 mg, yield 72%). ¹H-NMR (400 MHz, CDCl₃): δ=7.46-7.27 (m, 2H), 7.12 (d, J=7.6 Hz, 1H), 7.09 (d, J=7.6 Hz, 1H), 6.85 (d, J=9.3 Hz, 1H), 6.78 (d, J=2.4 Hz, 1H), 6.63 (d, J=2.9 Hz, 1H), 3.96 (s, 3H), 2.01 (s, 3H), 0.48 (s, 6H).

### Example 3

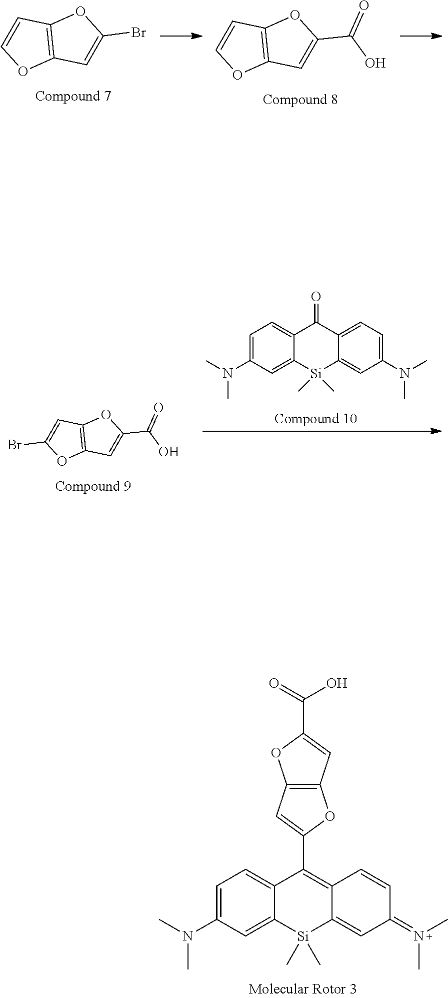

Compound 7 was synthesized by referring to the literature Org. Lett. 2009, 11, 14, 3144-3147. ¹H-NMR (400 MHz, CDCl₃): δ=7.87 (d, J=4.2 Hz, 1H), 7.13-7.08 (m, 2H).

Compound 8 was synthesized by referring to the literature Angew. Chem. Int. Ed. 2018, 57, 6910. ¹H-NMR (400 MHz, CDCl₃): δ=7.86 (d, J=4.2 Hz, 1H), 7.14-7.06 (m, 2H).

Synthesis of Compound 9: Add Compound 8 (2 g, 13 mmol) and 50 mL of acetonitrile into a 100 mL dry single-necked flask. After dissolution, under nitrogen protection, add N-bromosuccinimide (NBS) (2.6 g, 14.5 mmol) and ammonium acetate (51 mg, 0.66 mmol), and stir at room temperature. After the reaction is completed, the obtained solution is removed by distillation under reduced pressure, extracted with an ethyl acetate/water (EA/H₂O) system, washed with saturated sodium chloride (NaCl), dried over anhydrous sodium sulfate (Na₂SO₄), and the organic solvent is removed by distillation under reduced pressure. The obtained crude product can be purified by column chromatography to obtain the product with a yield of 69%. ¹H-NMR (400 MHz, CDCl₃): δ=7.84 (d, J=4.8 Hz, 1H), 6.99 (dd, J=4.8, 0.4 Hz, 1H).

Compound 10 was synthesized by referring to the literature J. Am. Chem. Soc. 2019, 141, 18, 7562-7571, with a yield of 39%. ¹H-NMR (400 MHz, CDCl₃): δ=8.40 (d, J=9.0 Hz, 2H), 6.84 (d, J=9.0, 2.8 Hz, 2H), 6.79 (d, J=2.8 Hz, 2H), 3.09 (s, 12H), 0.49 (s, 6H).

Synthesis of Molecular Rotor 3: Refer to the synthesis method of Molecular Rotor 1, with a yield of 39%. ¹H-NMR (400 MHz, CD₃OD): δ=12.86 (s, 1H), 7.16 (s, 2H), 6.88 (t, J=7.5 Hz, 2H), 6.81 (t, J=7.5 Hz, 1H), 6.47 (d, J=7.5 Hz, 2H), 6.08 (d, J=9.5 Hz, 1H), 3.66 (q, J=6.5 Hz, 12H), 0.66 (s, 6H).

### Example 4

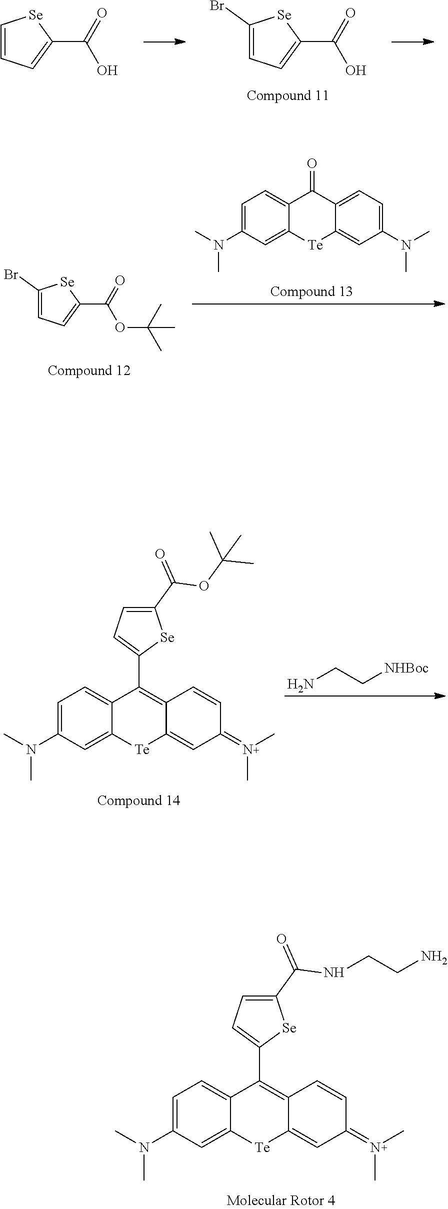

Synthesis of Compound 11: Refer to the synthesis method of Compound 9, with a yield of 56%. ¹H-NMR (400 MHz, CDCl₃): δ=7.89 (d, J=1.6 Hz, 1H), 7.45 (d, J=1.6 Hz, 1H).

Synthesis of Compound 12: Refer to the synthesis method of Compound 2, with a yield of 76% ¹H-NMR (400 MHz, CDCl₃): δ=7.86 (d, J=1.6 Hz, 1H), 7.43 (d, J=1.6 Hz, 1H), 1.53 (s, 9H).

Compound 13 was synthesized by referring to the literature Dalton Trans., 2019, 48, 5665-5673. ¹H-NMR (400 MHz, CDCl₃): δ=8.56 (d, J=9.1 Hz, 2H), 6.78 (d, J=2.5 Hz, 2H), 6.74 (dd, J=2.6, 9.1 Hz, 2H), 3.05 (s, 12H).

Synthesis of Compound 14: Refer to the synthesis method of Molecular Rotor 1, with a yield of 65%. ¹H-NMR (400 MHz, CDCl₃): δ=7.98(d, J=, 7.4 Hz, 1H), 7.39 (m, 2H), 7.21 (d, J=7.6 Hz, 1H), 7.09 (d, J=7.6 Hz, 1H), 6.88 (d, J=9.3 Hz, 1H), 6.82 (d, J=2.4 Hz, 1H), 6.63 (d, J=2.9 Hz, 1H), 3.09 (s, 12H), 1.56 (s, 9H).

Synthesis of Molecular Rotor 4: Dissolve Compound 14 (50 mg, 0.082 mmol) in 8 mL of anhydrous DCM, then add 2 mL of trifluoroacetic acid. After stirring at room temperature for 2 hours, remove the solvent by distillation under reduced pressure. Then dissolve the obtained solid in anhydrous DMF, and add N-Boc-ethylenediamine (14 mg, 0.09 mmol), the condensing agent PyBop (64 mg, 0.12 mmol), and 0.1 mL of triethylamine, and stir at room temperature for 1 hour. After the reaction is completed, remove the solvent by distillation under reduced pressure, extract with DCM, combine the organic phases, dry over anhydrous sodium sulfate, remove the solvent by distillation under reduced pressure. After purification by column chromatography, dissolve the obtained solid in 8 mL of anhydrous DCM, add 2 mL of trifluoroacetic acid, stir at room temperature for 2 hours, and remove the solvent by distillation under reduced pressure to obtain the product (40 mg, yield 82%). ¹H-NMR (400 MHz, DMSO-d₆): δ=7.98 (d, J=7.4 Hz, 1H), 7.39 (m, 2H), 7.21 (d, J=7.6 Hz, 1H), 7.09 (d, J=7.6 Hz, 1H), 6.88 (d, J=9.3 Hz, 1H), 6.82 (d, J=2.4 Hz, 1H), 6.63 (d, J=2.9 Hz, 1H), 3.46 (t, J=7.8 Hz, 2H), 3.09 (s, 12H), 2.46 (t, J=7.7 Hz, 2H).

### Example 5

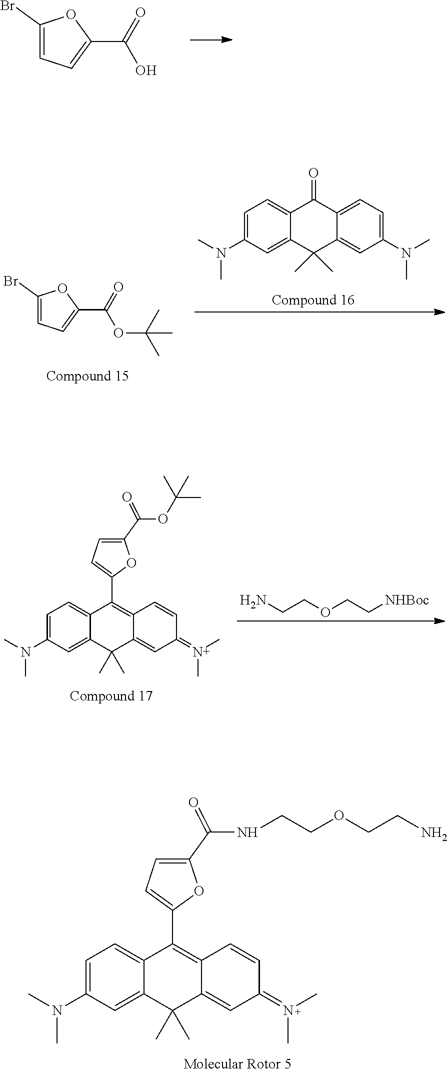

Synthesis of Compound 15: Refer to the synthesis method of Compound 2, with a yield of 65%. ¹H-NMR (400 MHz, DMSO-d₆): δ=7.88 (d, J=1.6 Hz, 1H), 7.56 (d, J=1.6 Hz, 1H), 1.51 (s, 9H).

Compound 16 was synthesized by referring to the literature J. Am. Chem. Soc. 2019, 141, 18, 7562-7571, with a yield of 29%. ¹H-NMR (400 MHz, CDCl₃): δ=8.27 (dd, J=7.9, 0.6 Hz, 2H), 6.72-6.79 (m, 4H), 3.09 (s, 12H), 1.72 (s, 6H).

Synthesis of Compound 17: Refer to the synthesis method of Molecular Rotor 1, with a yield of 55%. ¹H-NMR (400 MHz, CDCl₃): δ=7.68 (d, J=6.9 Hz, 1H), 7.29 (m, 2H), 7.10 (d, J=7.5 Hz, 1H), 7.02 (d, J=6.6 Hz, 1H), 6.78 (d, J=8.3 Hz, 1H), 6.68 (d, J=2.5 Hz, 1H), 6.43 (d, J=2.6 Hz, 1H), 3.09 (s, 12H), 1.56 (s, 9H), 1.38 (s, 6H).

Synthesis of Molecular Rotor 5: Dissolve Compound 17 (40 mg, 0.087 mmol) in 8 mL of anhydrous dichloromethane (DCM), then add 2 mL of trifluoroacetic acid. After stirring at room temperature for 2 hours, remove the solvent by distillation under reduced pressure. Then dissolve the obtained solid in anhydrous N,N-dimethylformamide (DMF), and add tert-butyl [2-(2-aminoethoxy)ethyl]carbamate (20 mg, 0.096 mmol), the condensing agent PyBop (68 mg, 0.13 mmol), and 0.1 mL of triethylamine, and stir at room temperature for 1 hour. After the reaction is completed, remove the solvent by distillation under reduced pressure, extract with DCM, combine the organic phases, dry over anhydrous sodium sulfate, remove the solvent by distillation under reduced pressure. After purification by column chromatography, dissolve the obtained solid in 8 mL of anhydrous DCM, add 2 mL of trifluoroacetic acid, stir at room temperature for 2 hours, and remove the solvent by distillation under reduced pressure to obtain the product (29 mg, yield 67%). ¹H-NMR (400 MHz, CDCl₃): δ=7.68 (d, J=6.9 Hz, 1H), 7.29 (m, 2H), 7.10 (d, J=7.5 Hz, 1H), 7.02 (d, J=6.6 Hz, 1H), 6.78 (d, J=8.3 Hz, 1H), 6.68 (d, J=2.5 Hz, 1H), 6.43 (d, J=2.6 Hz, 1H), 3.66 (t, J=7.8 Hz, 4H), 3.26 (t, J=6.9 Hz, 4H), 3.09 (s, 12H), 1.38 (s, 6H).

### Example 6

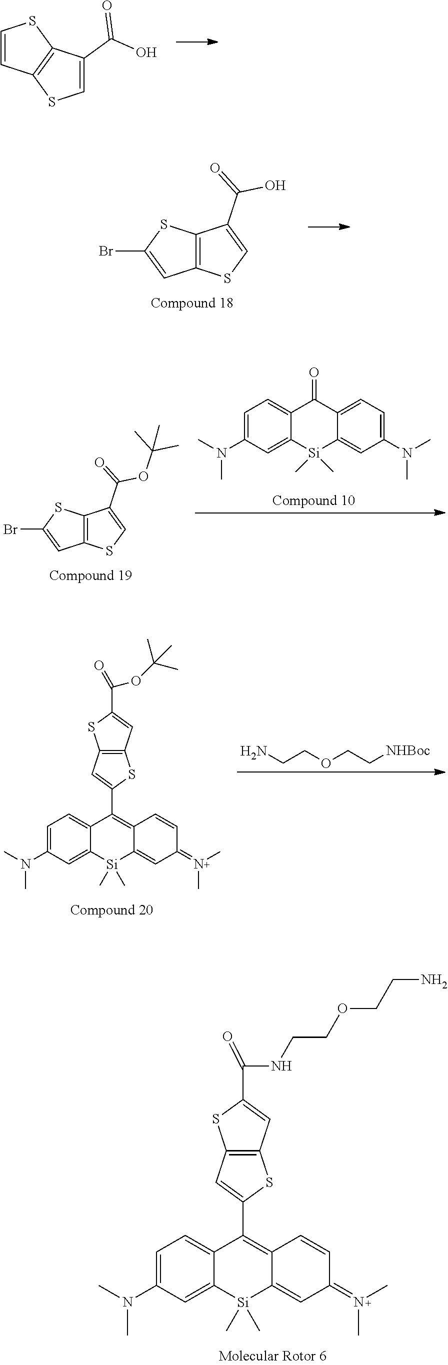

Synthesis of Compound 18: Refer to the synthesis method of Compound 9, with a yield of 92%. ¹H-NMR (400 MHz, DMSO-D₆): 8=10.63 (s, 1H), 8.46 (s, 1H), 7.82 (s, 1H).

Synthesis of Compound 19: Refer to the synthesis method of Compound 2, with a yield of 95%. ¹H-NMR (400 MHz, DMSO-D₆): 8=8.44 (s, 1H), 7.84 (s, 1H), 1.42 (s, 9H).

Synthesis of Compound 20: Refer to the synthesis method of Molecular Rotor 1, with a yield of 73%. ¹H-NMR (400 MHz, CDCl₃): δ=8.35 (dd, J=7.9, 1.7 Hz, 1H), 7.40 (d, J=7.9 Hz, 1H), 7.30 (d, J=2.8 Hz, 2H), 6.93 (d, J=9.4 Hz, 2H), 6.74 (dd, J=9.5 Hz, 2H), 3.26 (s, 12H), 1.56 (s, 9H), 1.38 (s, 6H).

Synthesis of Molecular Rotor 6: Refer to the synthesis method of Molecular Rotor 5, with a yield of 73%. ¹H-NMR (400 MHz, CDCl₃): δ=8.35 (dd, J=7.9, 1.7 Hz, 1H), 7.40 (d, J=7.9 Hz, 1H), 7.30 (d, J=2.8 Hz, 2H), 6.93 (d, J=9.4 Hz, 2H), 6.74 (dd, J=9.5 Hz, 2H), 3.75 (t, J=9.6 Hz, 4H), 3.36 (t, J=9.4 Hz, 4H), 3.00 (s, 12H), 1.32 (s, 6H).

### Example 7

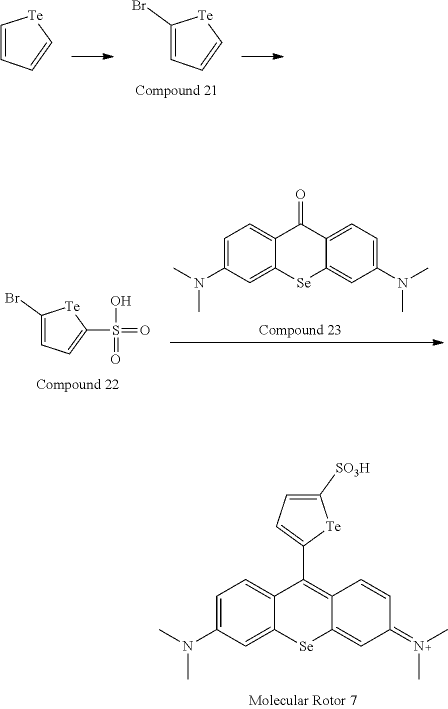

Synthesis of Compound 21: Add tellurophene (1 g, 5.6 mmol) and 40 mL of acetonitrile into a 100 mL dry single-necked flask. After the tellurophene is dissolved, under nitrogen protection, add N-bromosuccinimide (NBS) (1.1 g, 6.2 mmol) and ammonium acetate (43 mg, 0.56 mmol), and stir at room temperature. After the reaction is completed, the obtained solution is removed by distillation under reduced pressure, extracted with an ethyl acetate/water (EA/H₂O) system, washed with saturated sodium chloride (NaCl), dried over anhydrous sodium sulfate (Na₂SO₄), and the organic solvent is removed by distillation under reduced pressure. The obtained crude product can be purified by column chromatography to obtain the product (1.2 g, yield 80.6%). ¹H-NMR (400 MHz, CDCl₃): δ=6.98 (s, J=9.2 Hz, 1H), 6.81 (dd, J=8.6 Hz, 1H), 6.74 (d, J=8.4 Hz, 1H).

Synthesis of Compound 22: In an ice bath and dry environment, slowly add trifluoroacetic anhydride (30 mL, 0.21 mol) to a sulfuric acid (9.81 g, 0.1 mol) solution and stir for 3 hours to completely dissolve the sulfuric acid. After the solution turns light brown, add Compound 21 (25.9 g, 0.1 mol) dropwise to the above mixture. After stirring for 10 minutes, add cold water to the system to precipitate the solid. After removing the liquid by distillation under reduced pressure, the crude product is obtained. The obtained product can be purified by column chromatography to obtain the product (34.8 g, yield 98%). ¹H-NMR (400 MHz, CDCl₃): δ=7.03 (s, J=7.0 Hz, 1H), 6.96 (dd, J=8.2 Hz, 1H).

Synthesis of Compound 23: Refer to the literature J. Org. Chem. 2003, 68, 3344-3347 for synthesis, with a yield of 23%. ¹H-NMR (400 MHz, CDCl₃): δ=8.42 (d, J=9.2 Hz, 2H), 6.81 (dd, J=2.4, 9.2 Hz, 2H), 6.74 (d, J=2.4 Hz, 2H), 3.11 (s, 12H).

Synthesis of Molecular Rotor 7: Refer to the synthesis method of Molecular Rotor 1, with a yield of 52%. ¹H-NMR (400 MHz, CDCl₃): δ=8.67 (s, J=8.5 Hz, 1H), 8.56 (d, J=9.1 Hz, 2H), 7.60 (s, J=6.5 Hz, 1H), 6.83 (dd, J=2.6, 9.2 Hz, 2H), 6.76 (d, J=2.4 Hz, 2H), 3.07 (s, 12H).

### Example 8

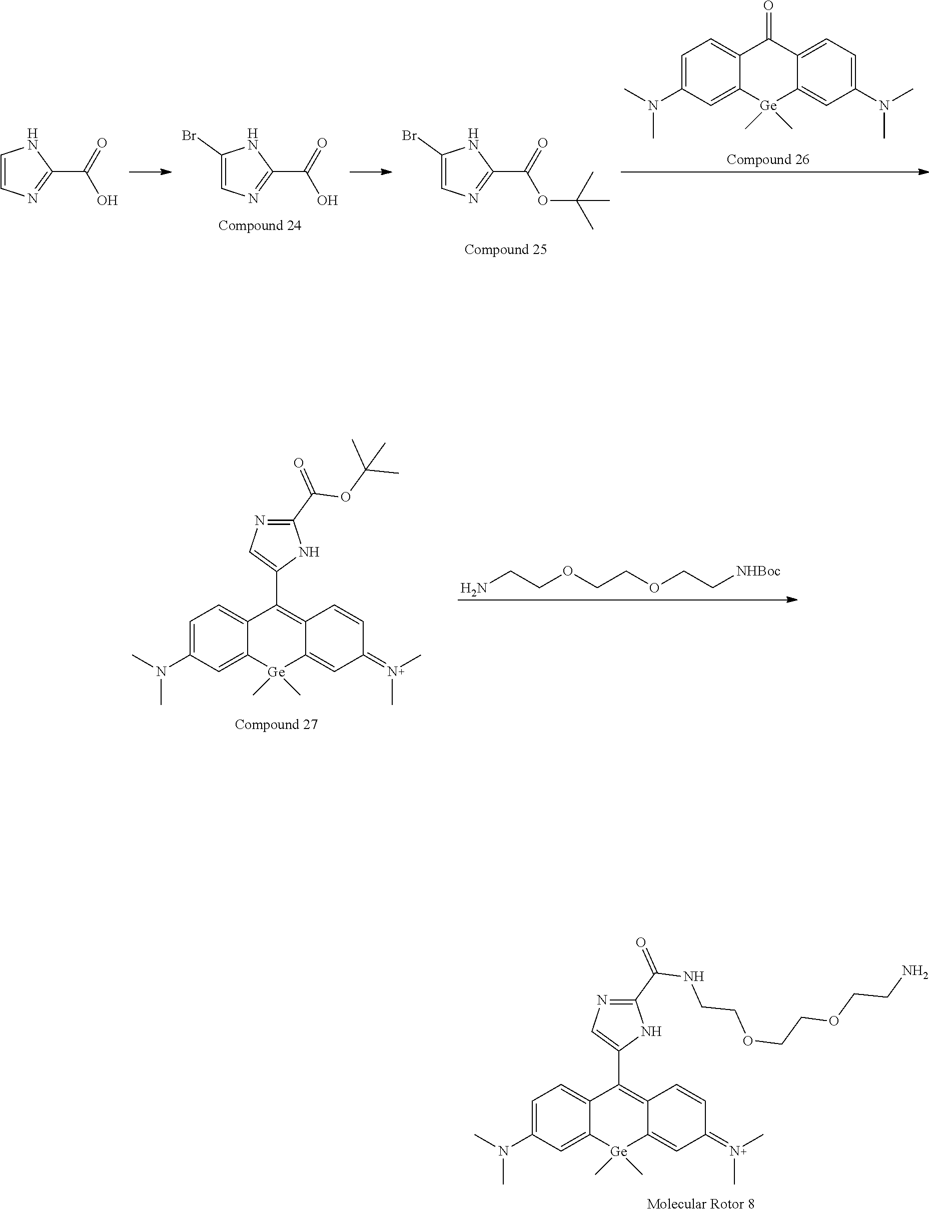

Synthesis of Compound 24: Refer to the synthesis method of Compound 9, with a yield of 65%. ¹H-NMR (400 MHz, DMSO-d₆): δ=12.97 (s, 1H), 12.75 (s, 1H), 7.82 (s, 1H).

Synthesis of Compound 25: Refer to the synthesis method of Compound 2, with a yield of 76%. ¹H-NMR (400 MHz, DMSO-d₆): δ=12.86 (s, 1H), 7.86 (s, 1H), 1.51 (s, 9H).

Synthesis of Compound 26: Refer to the literature Chem. Eur. J. 2017,23,12114-12119 for synthesis, with a yield of 72%. ¹H-NMR (400 MHz, CDCl₃): δ=8.42 (d, J=9.0 Hz, 2H), 6.80 (dd, J=9.1, 2.8 Hz, 2H), 6.72 (d, J=2.8 Hz, 2H), 3.08 (s, 12H), 0.60 (s, 6H).

Synthesis of Compound 27: Refer to the synthesis method of Molecular Rotor 1, with a yield of 34%. ¹H-NMR (400 MHz, CDCl₃): δ=7.85 (dd, J=6.8, 1.9 Hz, 1H), 7.54 (d, J=7.6, 2.2 Hz, 1H), 7.06 (d, J=2.8 Hz, 2H), 6.73 (d, J=9.4 Hz, 2H), 6.64 (dd, J=9.2 Hz, 1H), 3.06 (s, 12H), 1.56 (s, 9H), 0.68 (s, 6H).

Synthesis of Molecular Rotor 8: Dissolve Compound 27 (50 mg, 0.096 mmol) in 8 mL of anhydrous DCM, then add 2 mL of trifluoroacetic acid. After stirring at room temperature for 2 hours, remove the solvent by distillation under reduced pressure. Then dissolve the obtained solid in anhydrous DMF, and add tert-butyl 2-(2-(2-aminoethoxy)ethoxy)ethylcarbamate (26 mg, 0.011 mmol), the condensing agent PyBop (75 mg, 0.14 mmol), and 0.1 mL of triethylamine, and stir at room temperature for 1 hour. After the reaction is completed, remove the solvent by distillation under reduced pressure, extract with DCM, combine the organic phases, dry over anhydrous sodium sulfate, remove the solvent by distillation under reduced pressure. After purification by column chromatography, dissolve the obtained solid in 8 mL of anhydrous DCM, add 2 mL of trifluoroacetic acid, stir at room temperature for 2 hours, and remove the solvent by distillation under reduced pressure to obtain the product (40 mg, yield 70%). ¹H-NMR (400 MHz, CDCl₃): δ=7.85 (dd, J=6.8, 1.9 Hz, 1H), 7.54 (d, J=7.6, 2.2 Hz, 1H), 7.06 (d, J=2.8 Hz, 2H), 6.73 (d, J=9.4 Hz, 2H), 6.64 (dd, J=9.2 Hz, 1H), 3.75 (t, J=9.6 Hz, 4H), 3.66 (t, J=9.4 Hz, 4H), 3.35 (t, J=8.7 Hz, 2H), 3.26 (t, J=9.4 Hz, 2H), 3.06 (s, 12H), 0.68 (s, 6H).

### Example 9

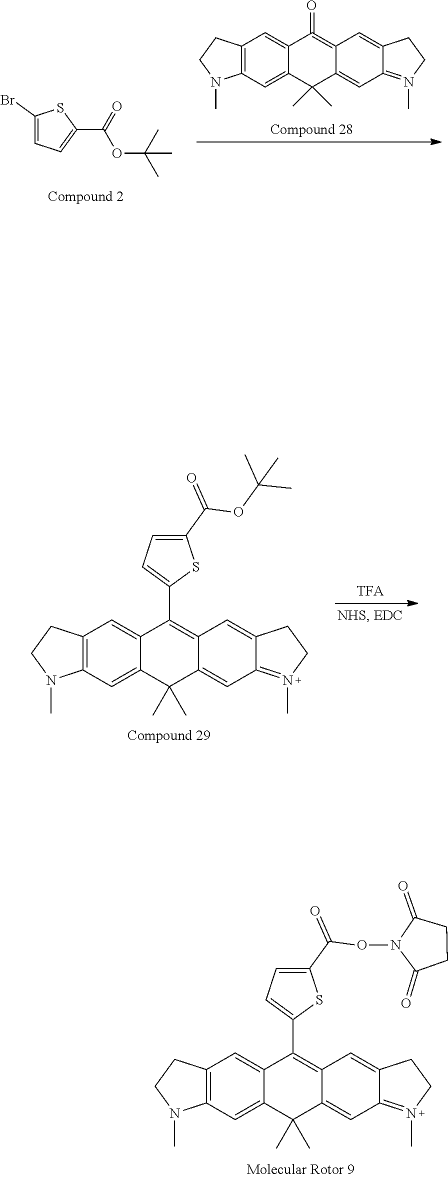

Synthesis of Compound 28: Refer to the literatures J. Am. Chem. Soc. 2012, 134, 5029-5031 and J. Am. Chem. Soc. 2019, 141, 7562-7571 for synthesis, with a yield of 22%. ¹H-NMR (400 MHz, CDCl₃): δ=7.42 (d, J=9.0 Hz, 2H), 6.80 (dd, J=9.1, 2.8 Hz, 2H), 3.47 (t, J=8.1 Hz, 4H), 3.05 (t, J=8.1 Hz, 4H), 2.90 (s, 6H), 0.44 (s, 6H).

Synthesis of Compound 29: Refer to the synthesis method of Molecular Rotor 1, with a yield of 82%. ¹H-NMR (400 MHz, CDCl₃): δ=7.9 (d, J=7.4 Hz, 1H), 7.34-7.36 (m, 2H), 6.80 (dd, J=9.1, 2.8 Hz, 2H), 6.06 (s, 2H), 3.34 (t, J=7.8 Hz, 4H), 3.09 (t, J=8.0 Hz, 4H), 2.90 (s, 6H), 1.56 (s, 9H), 0.44 (s, 6H).

Synthesis of Molecular Rotor 9: Dissolve Compound 29 (30 mg, 0.06 mmol) in 8 mL of anhydrous DCM, then add 2 mL of trifluoroacetic acid. After stirring at room temperature for 2 hours, remove the solvent by distillation under reduced pressure. Then dissolve the obtained solid in anhydrous DCM, add N-hydroxysuccinimide (NHS) (10 mg, 0.09 mmol), 1-Ethyl-3-(3-dimethylaminopropyl)carbodiimide (EDC) (17 mg, 0.09 mmol), and 0.1 mL of triethylamine, and stir at room temperature for 2 hours. After the reaction is completed, remove the solvent by distillation under reduced pressure, and purify by column chromatography to obtain the product (25 mg, yield 76%). ¹H-NMR (400 MHz, CDCl₃): δ=7.9 (d, J=7.4 Hz, 1H), 7.34-7.36 (m, 2H), 6.80 (dd, J=9.1, 2.8 Hz, 2H), 6.06 (s, 2H), 3.34 (t, J=7.8 Hz, 4H), 3.09 (t, J=8.0 Hz, 4H), 2.90 (s, 6H), 2.56 (t, J=7.8 Hz, 4H), 0.44 (s, 6H).

### Example 10

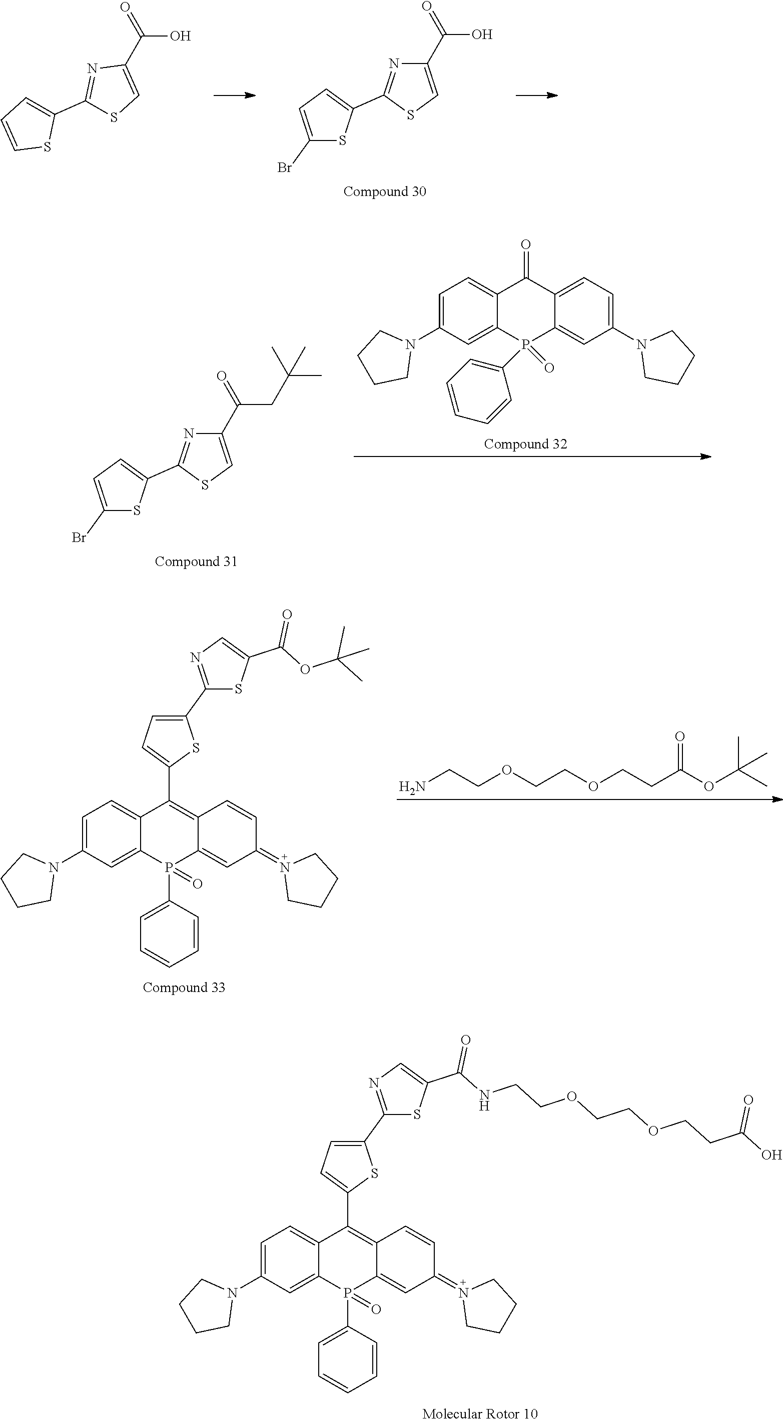

Synthesis of Compound 30: Refer to the synthesis method of Compound 9, with a yield of 72%. ¹H-NMR (400 MHz, DMSO-D₆): 8=12.63 (s, 1H), 9.46 (s, 1H), 7.82 (d, J=7.6, 2.3 Hz, 1H), 6.95 (d, J=8.6, 2.4 Hz, 1H).

Synthesis of Compound 31: Refer to the synthesis method of Compound 2, with a yield of 88%. ¹H-NMR (400 MHz, DMSO-D₆): 8=9.44 (s, 1H), 7.79 (d, J=7.6, 2.3 Hz, 1H), 6.96 (d, J=8.6, 2.4 Hz, 1H), 1.42 (s, 9H).

Synthesis of Compound 32: Refer to the literature Chem. Eur J. 2020, 26, 7912-7917 for synthesis, with a yield of 61%. ¹H-NMR (400 MHz, CDCl₃): δ=8.31 (dd, J=8.9, 5.8 Hz, 2H), 7.64-7.58 (m, 2H), 7.37-7.28 (m, 3H), 7.00 (dd, J=14.7, 2.4 Hz, 2H), 6.72 (dd, J=9.2, 2.4 Hz, 2H), 3.43-3.32 (m, 8H), 2.03-1.99 (m, 8H).

Synthesis of Compound 33: Refer to the synthesis method of Molecular Rotor 1, with a yield of 78%. ¹H-NMR (400 MHz, CDCl₃): δ=8.40 (dd, J=8.8, 5.8 Hz, 2H), 7.87 (s, 1H), 7.61 (m, 2H), 7.39 (s, 1H), 7.34 (m, 3H), 7.02 (dd, J=14.6, 2.4 Hz, 2H), 6.72 (dd, J=9.2, 2.4 Hz, 2H), 3.40 (m, 8H), 2.01 (m, 8H).

Synthesis of Molecular Rotor 10: Dissolve Compound 33 (25 mg, 0.041 mmol) in 8 mL of anhydrous DCM, then add 2 mL of trifluoroacetic acid. After stirring at room temperature for 2 hours, remove the solvent by distillation under reduced pressure. Then dissolve the obtained solid in anhydrous DMF, and add tert-butyl 9-amino-4,7-dioxononanoate (11 mg, 0.045 mmol), the condensing agent PyBop (32 mg, 0.062 mmol), and 0.1 mL of triethylamine, and stir at room temperature for 1 hour. After the reaction is completed, remove the solvent by distillation under reduced pressure, extract with DCM, combine the organic phases, dry over anhydrous sodium sulfate, remove the solvent by distillation under reduced pressure. After purification by column chromatography, dissolve the obtained solid in 8 mL of anhydrous DCM, add 2 mL of trifluoroacetic acid, stir at room temperature for 2 hours, and remove the solvent by distillation under reduced pressure to obtain the product (19.3 mg, yield 66%). ¹H-NMR (400 MHz, CDCl₃): δ=8.35 (s, 1H), 8.25 (s, 1H), 8.02 (d, J=7.6 Hz, 1H), 7.34 (m, 3H), 7.08 (dd, J=14.6, 2.4 Hz, 2H), 6.76 (dd, J=9.2, 2.4 Hz, 2H), 7.22 (d, J=7.3 Hz, 1H), 7.16 (s, 2H), 6.62 (s, 2H), 3.83 (t, J=8.1 Hz, 4H), 3.23 (s, 2H), 2.93 (t, J=8.1 Hz, 4H), 2.10 (s, 2H), 3.40 (m, 8H), 2.01 (m, 8H).

### Example 11

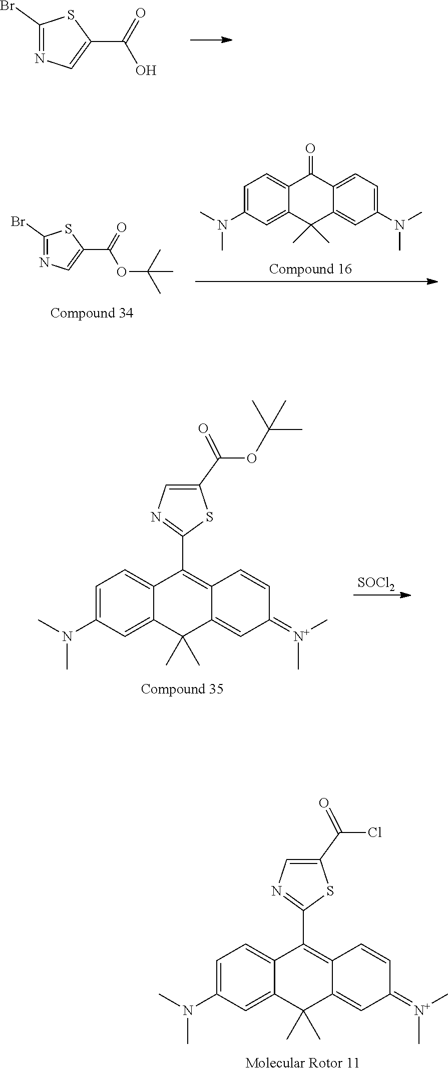

Synthesis of Compound 34: Refer to the synthesis method of Compound 2, with a yield of 65%. ¹H-NMR (400 MHz, DMSO-d₆): δ=7.82 (s, 1H), 1.51 (s, 9H).

Synthesis of Compound 35: Refer to the synthesis method of Molecular Rotor 1, with a yield of 78%. ¹H-NMR (400 MHz, CDCl₃): δ=8.25 (d, J=8.4 Hz, 1H), 7.68 (d, J=8.4 Hz, 1H), 7.61 (t, J=7.2 Hz, 1H), 7.03 (d, J=7.2 Hz, 2H), 6.92 (s, 2H), 1.63 (s, 9H), 1.37 (s, 12H), 0.45 (s, 6H).

Synthesis of Molecular Rotor 11: Dissolve Compound 35 (257 mg, 0.54 mmol) in 8 mL of anhydrous DCM, then add 2 mL of trifluoroacetic acid. After stirring at room temperature for 2 hours, remove the solvent by distillation under reduced pressure. Then dissolve the obtained solid in 100 mL of anhydrous DCM, add a catalytic amount of DMF (3 drops), cool the reaction system to 0° C. and stir for 5 minutes. Then, add thionyl chloride (77 mg, 6.5 mmol) dropwise and keep stirring at 85° C. for 5 hours. After the obtained mixture is rotary evaporated to remove the solvent, the product can be obtained, with a yield of 79%. ¹H-NMR (400 MHz, CDCl₃): δ=8.36 (d, J=8.4 Hz, 1H), 7.78 (d, J=8.4 Hz, 1H), 7.72 (t, J=7.2 Hz, 1H), 7.14 (d, J=7.2 Hz, 2H), 6.88 (s, 2H), 1.39 (s, 12H), 0.44 (s, 6H).

### Example 12

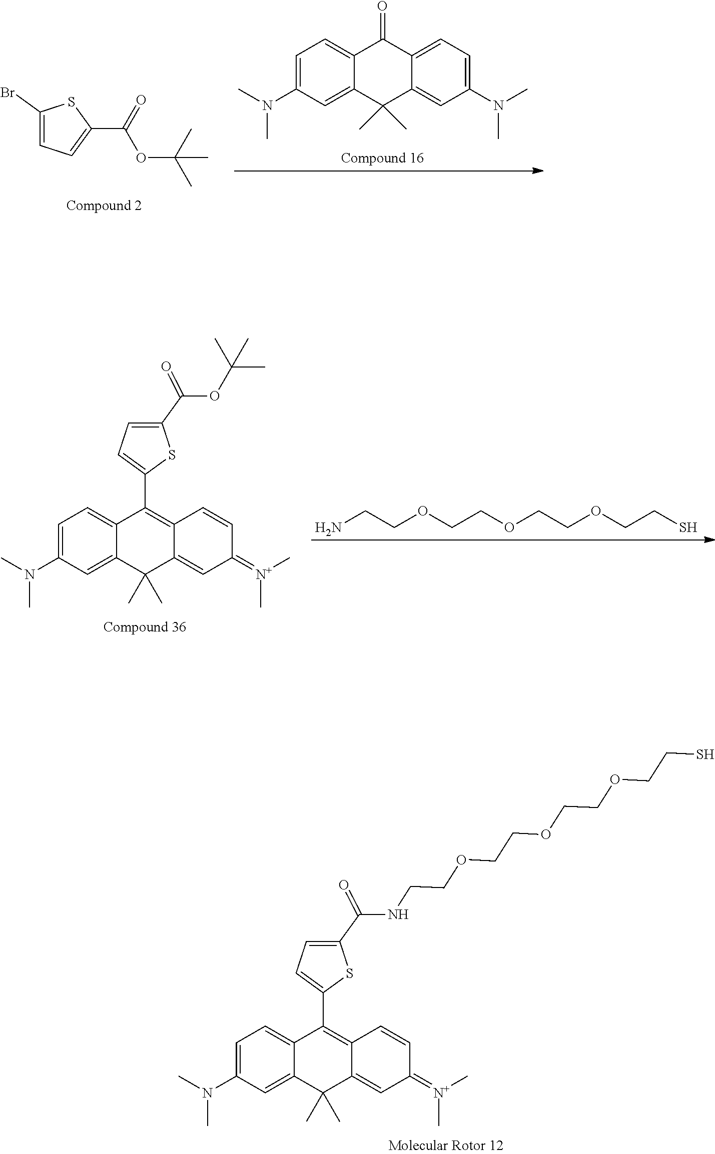

Synthesis of Compound 36: Refer to the synthesis method of Molecular Rotor 1, with a yield of 55%. ¹H-NMR (400 MHz, CDCl₃): δ=7.80 (d, J=6.9 Hz, 1H), 7.30 (m, 2H), 7.21 (d, J=7.5 Hz, 1H), 7.13 (d, J=6.6 Hz, 1H), 6.89 (d, J=8.3 Hz, 1H), 6.79 (d, J=2.5 Hz, 1H), 6.54 (d, J=2.6 Hz, 1H), 3.10 (s, 12H), 1.67 (s, 9H), 1.49 (s, 6H).

Synthesis of Molecular Rotor 12: Dissolve Compound 36 (40 mg, 0.084 mmol) in 8 mL of anhydrous dichloromethane (DCM), then add 2 mL of trifluoroacetic acid. Stir at room temperature for 2 hours, and then remove the solvent by distillation under reduced pressure. After that, dissolve the obtained solid in anhydrous N,N-dimethylformamide (DMF), add mercapto-triethylene glycol-amine (19 mg, 0.093 mmol), the condensing agent PyBop (66 mg, 0.13 mmol), and 0.1 mL of triethylamine, and stir at room temperature for 1 hour. After the reaction is completed, remove the solvent by distillation under reduced pressure, extract with DCM, combine the organic phases, dry over anhydrous sodium sulfate, remove the solvent by distillation under reduced pressure. After purification by column chromatography, the product can be obtained (32, yield 62%). ¹H-NMR (400 MHz, CDCl₃): δ=8.36 (s, 1H), 8.25 (d, J=7.5 Hz, 1H), 7.89 (d, J=6.9 Hz, 1H), 7.30 (s, 1H), 7.21 (d, J=7.5 Hz, 1H), 7.13 (d, J=6.6 Hz, 1H), 6.89 (d, J=8.3 Hz, 1H), 6.79 (d, J=2.5 Hz, 1H), 6.54 (d, J=2.6 Hz, 1H), 3.83 (t, J=8.1 Hz, 4H), 3.23 (s, 2H), 3.10 (s, 12H), 2.93 (t, J=8.1 Hz, 4H), 2.10 (s, 2H), 1.49 (s, 6H).

### Example 13

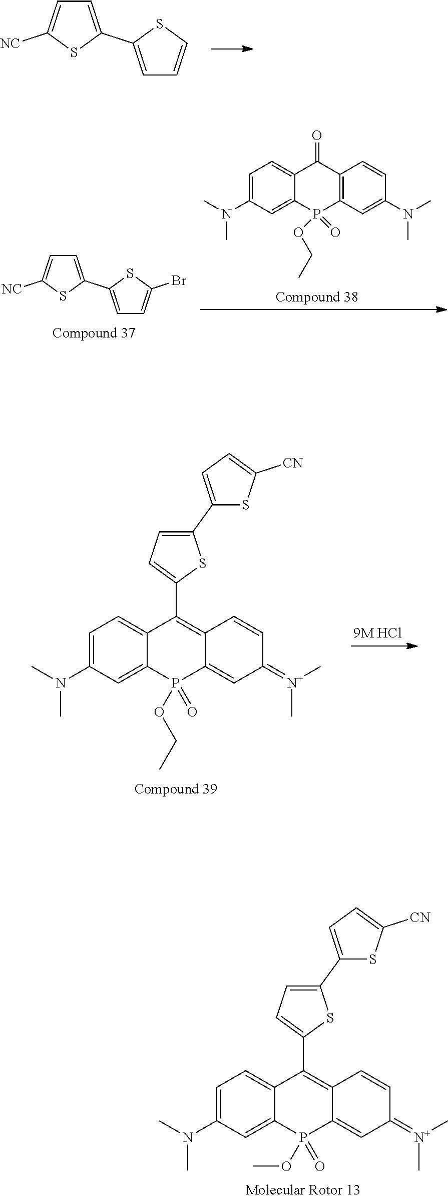

Synthesis of Compound 37: Dissolve 5-(thiophen-2-yl)thiophene-2-carbonitrile (4.77 g, 30 mmol) in 20 mL of N,N-dimethylformamide (DMF), add N-bromosuccinimide (NBS) (5.34 g, 30 mmol) in batches, and then stir overnight. After the reaction is completed, pour the above reaction system into cold water. Filter and collect the formed solid, wash it with clean water and dry it to obtain Compound 37 with a yield of 95%. ¹H-NMR (400 MHz, DMSO-d₆): δ=7.88 (d, J=3.9 Hz, 1H), 7.41 (d, J=3.9 Hz, 1H), 7.34 (d, J=3.9 Hz, 1H), 7.26 (d, J=3.9 Hz, 1H).

Synthesis of Compound 38: Refer to the literature Chem. Commun., 2016, 52, 12290-12293 for synthesis, with a yield of 35%. ¹H-NMR (400 MHz, CDCl₃): δ=8.28 (tq, J=6.8, 2.6, 2.0 Hz, 2H), 7.23 (ddd, J=15.2, 4.1, 2.6 Hz, 2H), 6.91 (ddd, J=9.1, 4.1, 2.6 Hz, 2H), 3.90 (ddd, J=9.9, 5.6, 2.8 Hz, 2H), 3.21-3.09 (m, 12H), 1.24-1.11 (m, 3H).

Synthesis of Compound 39: Refer to the synthesis method of Molecular Rotor 1, with a yield of 49%. ¹H-NMR (400 MHz, DMSO-d₆): δ=7.65-7.37 (m, 5H), 7.18 (t, J=7.8 Hz, 1H), 7.04 (td, J=10.3, 6.8 Hz, 2H), 6.83 (dd, J=9.7, 2.8 Hz, 2H), 4.09 (dp, J=9.6, 7.2 Hz, 2H), 3.38 (s, 12H), 1.24 (dt, J=18.1, 7.0 Hz, 3H).

Synthesis of Molecular Rotor 13: Dissolve the above-obtained Compound 39 (20 mg, 0.038 mmol) in a hydrochloric acid (HCl) (9 M, 2 mL) solution, and heat the reaction system under reflux and stir overnight. After the reaction is completed, remove the solvent by distillation under reduced pressure, and recrystallize the obtained solid to obtain the pure product (16 mg, yield 87%). ¹H-NMR (400 MHz, DMSO-d₆): δ=7.53-7.34 (m, 3H), 7.24 (d, J=14.7 Hz, 2H), 7.12 (d, J=7.3 Hz, 1H), 6.90-6.70 (m, 4H), 3.30 (s, 12H).

### Example 14

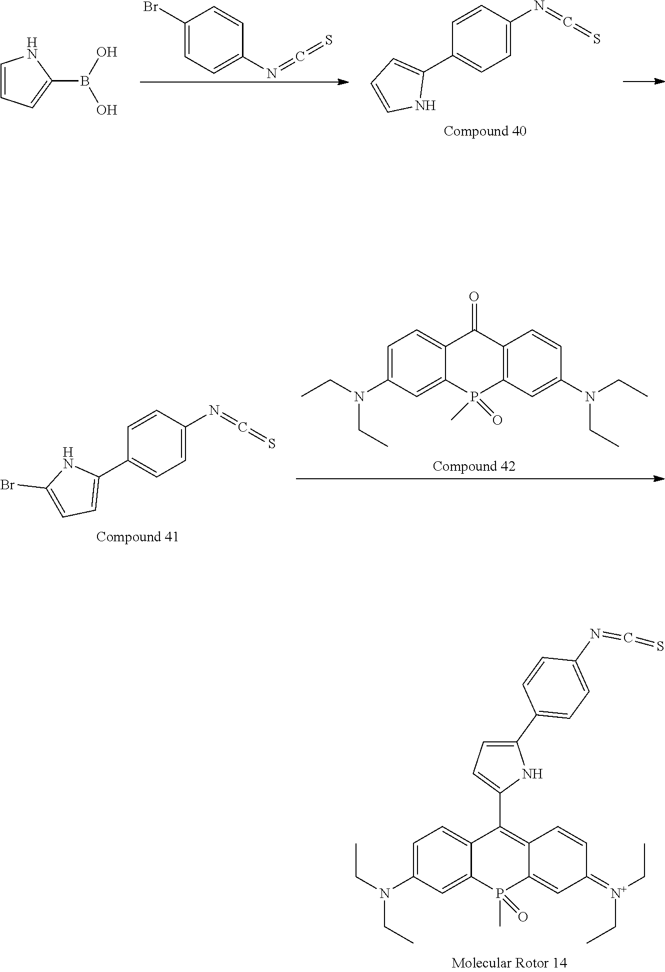 [SMILES: OB(O)c1ccc[nH]1 ; S=C=Nc1ccc(Br)cc1 ; S=C=Nc1ccc(-c2ccc[nH]2)cc1 ; S=C=Nc1ccc(-c2ccc(Br)[nH]2)cc1 ; CCN(CC)c1ccc2c(c1)P(C)(=O)c1cc(N(CC)CC)ccc1C2=O ; CCN(CC)c1ccc2c(c1)P(C)(=O)C1=CC(=[N+](CC)CC)C=CC1=C2c1ccc(-c2ccc(N=C=S)cc2)[nH]1]

Synthesis of Compound 40: In a 100 mL three-necked flask, add 2-pyrrole boronic acid (0.93 g, 8.4 mmol), 4-bromophenyl isothiocyanate (1.5 g, 7 mmol), sodium carbonate (Na₂CO₃) (1.86 g, 17.5 mmol), and a mixed solvent of 50 mL (toluene:ethanol:water=1:1:1). Then add tetrakis(triphenylphosphine)palladium (0.4 g, 0.35 mmol), and heat the reaction system to 80° C. under nitrogen protection and stir under reflux for 6 hours. After the reaction is completed, cool the reaction system to room temperature. Quench the reaction with ammonium chloride (NH₄Cl), extract with ethyl acetate (EA), combine the organic phases, dry over anhydrous sodium sulfate, remove the solvent by distillation under reduced pressure, and purify by column chromatography to obtain the product (1.23 g, yield 88%). ¹H-NMR (400 MHz, CDCl₃): δ=7.58-7.56 (m, 1H), 7.41-7.34 (m, 3H), 7.29-7.26 (m, 2H), 7.13 (t, J=4.4 Hz, 1H).

Synthesis of Compound 41: Refer to the synthesis method of Compound 9, with a yield of 74%. ¹H-NMR (400 MHz, CDCl₃): δ=7.47-7.37 (m, 3H), 7.27 (d, J=7.8 Hz, 1H), 7.16 (s, 1H), 7.14 (d, J=8.0 Hz, 1H).

Synthesis of Compound 42: Refer to the literature Chem. Eur J. 2015, 21, 16754-16758 for synthesis, with a yield of 38%. ¹H-NMR (400 MHz, CDCl₃): δ=8.29 (d, J=9.0 Hz, 1H), 8.26 (d, J=9.0 Hz, 1H), 7.26 (d, J=3.0 Hz, 1H), 7.21 (d, J=2.7 Hz, 1H), 6.90 (dd, J=9.0, 2.4 Hz, 2H), 3.51 (q, J=7.2 Hz, 8H), 1.88 (d, J=13.4 Hz, 3H), 1.25 (t, J=7.2 Hz, 12H).

Synthesis of Molecular Rotor 14: Refer to the synthesis method of Molecular Rotor 1, with a yield of 20%. ¹H-NMR (400 MHz, CDCl₃): δ=7.78-7.75 (m, 2H), 7.66-7.64 (m, 3H), 7.42-7.38 (m, 2H), 7.26 (d, J=2.4 Hz, 1H), 6.99 (d, J=2.4 Hz, 2H), 6.87 (d, J=2.4 Hz, 2H), 3.88-3.78 (m, 8H), 2.06 (d, J=13.6 Hz, 3H), 1.38 (t, J=6.0 Hz, 12H).

### Example 15

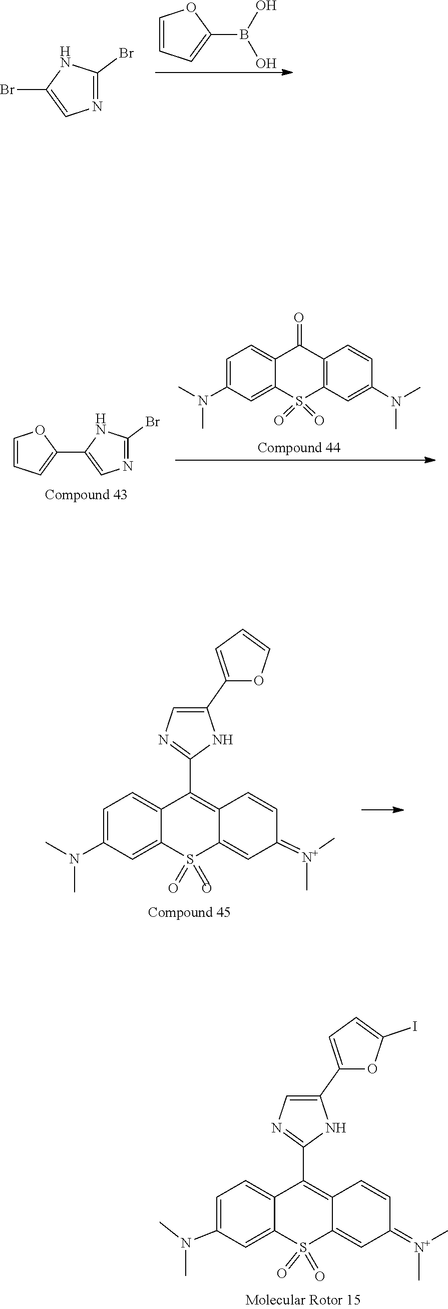

Synthesis of Compound 43: Refer to the synthesis method of Compound 40, with a yield of 84%. ¹H-NMR (400 MHz, DMSO-d₆): δ=12.25 (s, 1H), 7.71 (s, 1H), 7.58 (s, 1H), 7.34 (s, 1H), 6.51-6.49 (m, 1H).

Synthesis of Compound 44: Refer to the literature ACS Appl. Mater Interfaces 2016, 8, 22953-22962 for synthesis, with a yield of 38%. ¹H-NMR (400 MHz, DMSO-d₆): δ=8.00 (d, J=9.0 Hz, 2H), 7.09 (m, 4H), 3.14 (s, 12H).

Synthesis of Compound 45: Refer to the synthesis method of Molecular Rotor 1, with a yield of 20%. ¹H-NMR (400 MHz, CDCl₃): δ=7.65 (m, 3H), 7.4 (d, J=4.8 Hz, 2H), 7.37 (t, J=7.8 Hz, 2H), 7.22 (t, J=7.8 Hz, 2H), 7.08 (s, 1H), 3.58 (s, 12H).

Synthesis of Molecular Rotor 15: Add Compound 45 (20 mg, 0.044 mmol) and 50 mL of acetonitrile into a 100 mL dry single-necked flask. After dissolution, under nitrogen protection, add N-iodosuccinimide (NIS) (11 mg, 0.049 mmol) and ammonium acetate (0.17 mg, 0.002 mmol), and stir at room temperature. After the reaction is completed, the obtained solution is removed by distillation under reduced pressure, extracted with an ethyl acetate/water (EA/H₂O) system, washed with saturated sodium chloride (NaCl), dried over anhydrous sodium sulfate (Na₂SO₄), and the organic solvent is removed by distillation under reduced pressure. The obtained crude product is purified by column chromatography, with a yield of 89%. ¹H-NMR (400 MHz, CDCl₃): δ=7.59 (d, J=2.4 Hz, 2H), 7.28 (s, 1H), 7.14 (m, 3H), 7.05 (dd, J=2.4, 7.8 Hz, 2H), 6.07 (d, J=12.6 Hz, 1H), 3.55 (s, 12H).

### Example 16

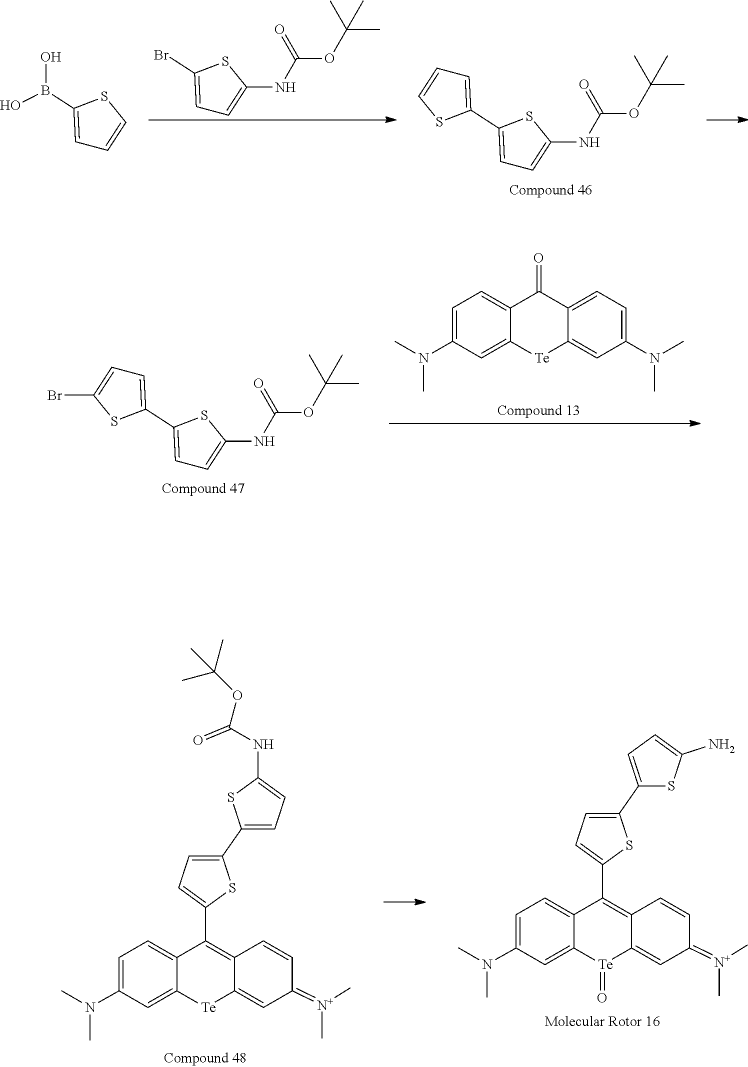

Synthesis of Compound 46: Refer to the synthesis method of Compound 40, with a yield of 85%. ¹H-NMR (400 MHz, CDCl₃): δ=7.86 (s, 1H), 7.66 (d, J=3.9 Hz, 1H), 7.35 (d, J=4.4 Hz, 1H), 7.25 (d, J=3.9 Hz, 1H), 7.08 (t, J=4.4 Hz, 1H), 1.53 (s, 9H).

Synthesis of Compound 47: Refer to the synthesis method of Compound 9, with a yield of 78%. ¹H-NMR (400 MHz, CDCl₃): δ=7.66 (d, J=3.9 Hz, 1H), 7.22 (d, J=3.8 Hz, 1H), 7.19 (d, J=3.9 Hz, 1H), 7.02 (d, J=3.8 Hz, 1H), 1.54 (s, 9H).

Synthesis of Compound 48: Refer to the synthesis method of Molecular Rotor 1, with a yield of 65%. ¹H-NMR (400 MHz, CDCl₃): δ=7.90 (d, J=2.9 Hz, 2H), 7.55-7.38 (m, 5H), 7.14 (d, J=7.3 Hz, 1H), 6.91 (dd, J=10.3, 2.9 Hz, 2H), 3.23 (s, 12H), 1.60 (s, 9H).

Synthesis of Molecular Rotor 16: Dissolve Compound 48 in 8 mL of anhydrous DCM, then add 2 mL of trifluoroacetic acid. Stir at room temperature for 2 hours, and then remove the solvent by distillation under reduced pressure. Then dissolve the solid in 2 mL of methanol, and slowly add an aqueous hydrogen peroxide solution (30-35%) dropwise in batches. Stir vigorously at room temperature. When the color of the solution changes, immediately dilute the reaction mixture with 3 mL of a 0.1% TFA aqueous solution. Then, the product can be obtained by purification through column chromatography, with a yield of 20%. ¹H-NMR (400 MHz, CDCl₃): δ=8.14 (d, J=2.9 Hz, 2H), 7.55-7.37 (m, 3H), 7.26 (d, J=9.5 Hz, 2H), 7.14 (d, J=8.0 Hz, 1H), 6.92 (dd, J=9.5, 2.9 Hz, 2H), 3.44 (s, 12H).

### Example 17

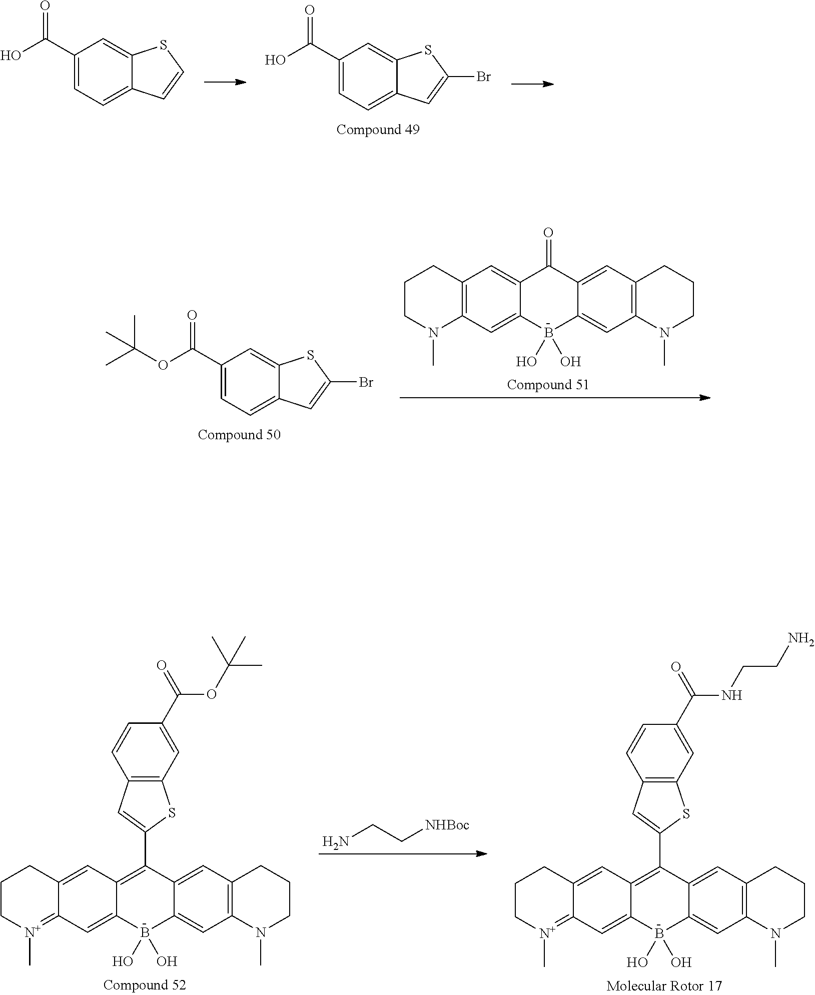

Synthesis of Compound 49: Refer to the synthesis method of Compound 9, with a yield of 89%. ¹H-NMR (400 MHz, DMSO-d₆): δ=13.01 (s, 1H), 8.64 (s, 1H), 8.01 (d, J=5.6 Hz, 1H), 7.98-7.92 (m, 1H), 7.55 (d, J=5.6 Hz, 1H).

Synthesis of Compound 50: Refer to the synthesis method of Compound 2, with a yield of 92%. ¹H-NMR (400 MHz, DMSO-d₆): δ=8.62 (s, 1H), 8.01 (d, J=5.6 Hz, 1H), 7.95 (m, 1H), 7.52 (d, J=5.6 Hz, 1H), 1.54 (s, 9H).

Synthesis of Compound 51: Refer to the literatures Angew. Chem. Int. Ed. 2017, 56, 4197-4200 and J. Am. Chem. Soc. 2012, 134, 5029-5031 for synthesis, with a yield of 18%. ¹H-NMR (400 MHz, CDCl₃): δ=7.41 (m, 2H), 7.22 (s, 2H), 3.60 (t, J=5.1 Hz, 4H), 3.33 (s, 6H), 2.48 (t, J=5.9 Hz, 4H), 1.91 (dt, J=5.9, 5.1 Hz, 4H).

Synthesis of Compound 52: Refer to the synthesis method of Molecular Rotor 1, with a yield of 65%. ¹H-NMR (400 MHz, CD₃OD): δ=7.43-7.26 (m, 3H), 7.22 (d, J=7.5 Hz, 1H), 7.08 (d, J=2.8 Hz, 2H), 6.42 (d, J=2.8 Hz, 1H), 6.39 (d, J=2.8 Hz, 1H), 3.64 (t, J=5.2 Hz, 4H), 3.35 (s, 6H), 2.49 (t, J=5.8 Hz, 4H), 1.92 (dt, J=5.9, 5.1 Hz, 4H), 1.54 (s, 9H).

Synthesis of Molecular Rotor 17: Refer to the synthesis method of Molecular Rotor 4, with a yield of 56%. ¹H-NMR (400 MHz, CD₃OD): δ=7.45-7.28 (m, 3H), 7.20 (d, J=7.5 Hz, 1H), 7.06 (d, J=2.8 Hz, 2H), 6.40 (d, J=2.8 Hz, 1H), 6.36 (d, J=2.8 Hz, 1H), 3.65 (t, J=5.2 Hz, 4H), 3.46 (t, J=7.8 Hz, 2H), 3.35 (s, 6H), 2.49 (t, J=5.8 Hz, 4H), 2.46 (t, J=7.7 Hz, 2H), 1.92 (dt, J=5.9, 5.1 Hz, 4H), 1.54 (s, 9H).

### Example 18

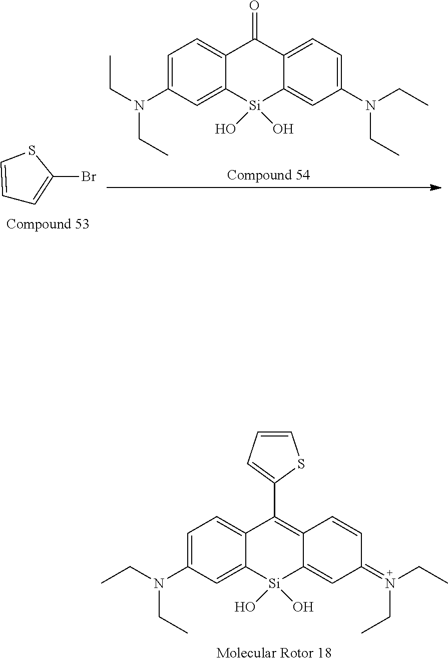

Synthesis of Compound 54: Refer to the literature Angew. Chem. Int. Ed. 2017, 56, 4197-420 for synthesis, with a yield of 20%. ¹H-NMR (400 MHz, CDCl₃): δ=8.26 (d, J=9.0 Hz, 2H), 7.21 (d, J=2.7 Hz, 2H), 6.90 (d, J=2.4 Hz, 2H), 3.51 (q, J=7.2 Hz, 8H), 1.25 (t, J=7.2 Hz, 12H).

Synthesis of Molecular Rotor 18: Refer to the synthesis method of Molecular Rotor 1, with a yield of 72%. ¹H-NMR (400 MHz, CDCl₃): δ=7.62 (d, J=3.0 Hz, 1H), 7.36 (d, J=7.0 Hz, 1H), 7.25 (s, 1H), 7.03 (d, J=7.4 Hz, 1H), 6.95 (s, 1H), 6.92 (s, 1H), 6.46 (d, J=2.9 Hz, 1H), 6.43 (d, J=3.0 Hz, 1H), 3.48 (q, J=7.2 Hz, 8H), 1.26 (t, J=7.2 Hz, 12H).

### Example 19

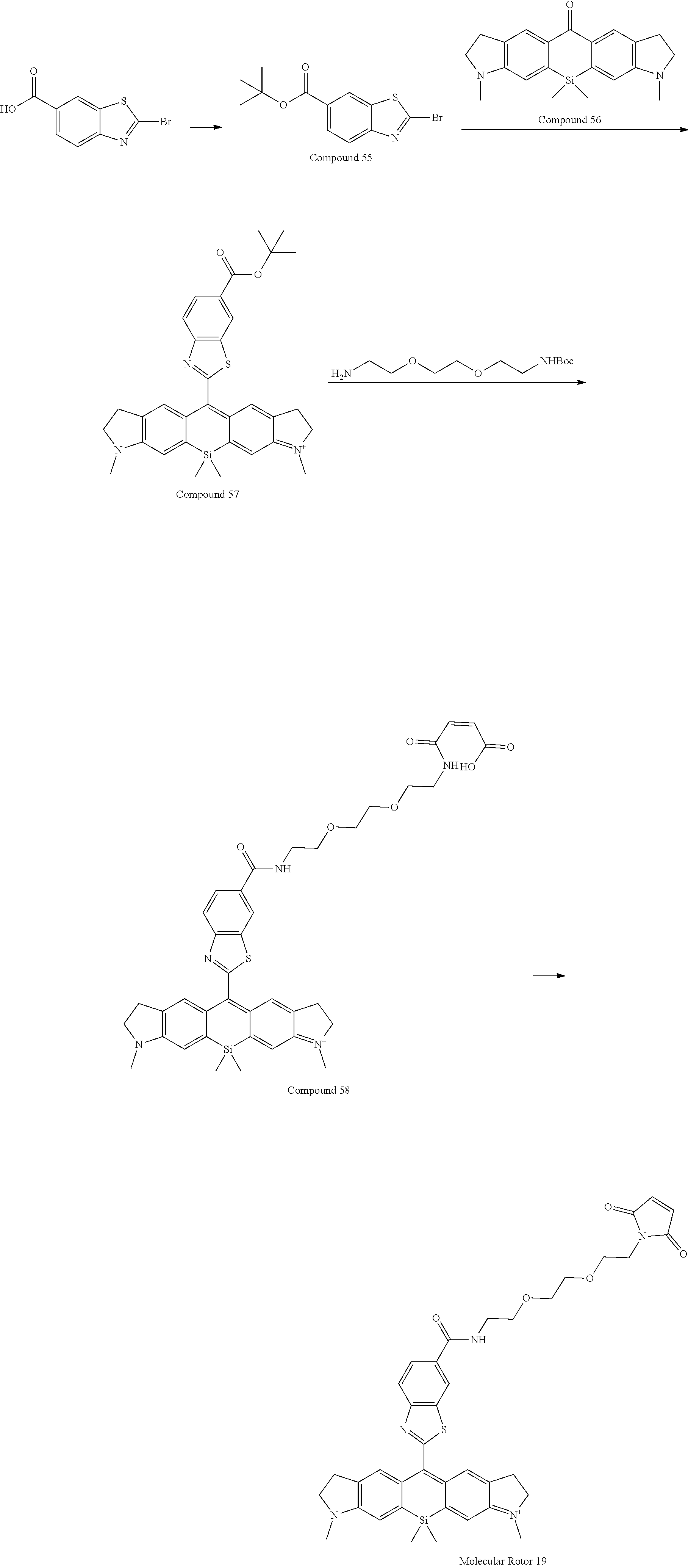

Synthesis of Compound 55: Refer to the synthesis method of Compound 2, with a yield of 87%. ¹H-NMR (400 MHz, DMSO-d₆): δ=8.62 (s, 1H), 8.01 (d, J=5.6 Hz, 1H), 7.95 (m, 1H), 1.54 (s, 9H).

Synthesis of Compound 56: Refer to the literature J. Am. Chem. Soc. 2012, 134, 5029-5031 for synthesis, with a yield of 17%. ¹H-NMR (400 MHz, CDCl₃): δ=8.21 (s, 2H), 6.51 (s, 2H), 3.47 (t, J=8.1 Hz, 4H), 3.05 (t, J=8.1 Hz, 4H), 2.90 (s, 6H), 0.44 (s, 6H).

Synthesis of Compound 57: Refer to the synthesis method of Molecular Rotor 1, with a yield of 36%. ¹H-NMR (400 MHz, CD₃OD): δ=8.05 (s, 1H), 8.02 (d, J=7.3 Hz, 2H), 7.22 (d, J=7.3 Hz, 1H), 7.16 (s, 1H), 6.62 (s, 2H), 3.83 (t, J=8.1 Hz, 4H), 3.23 (s, 6H), 2.93 (t, J=8.1 Hz, 4H), 1.50 (s, 9H), 0.58 (s, 3H), 0.56 (s, 3H).

Synthesis of Compound 58: Refer to the synthesis method of Molecular Rotor 8. Then dissolve the obtained molecule (0.2 g, 0.34 mmol) in 50 mL of DCM, add a catalytic amount of 4-dimethylaminopyridine (DMAP) and maleic anhydride (33 mg, 0.34 mmol), and stir the reaction system for 2 hours. After the reaction is completed, extract with DCM/aqueous sodium bicarbonate (NaHCO₃) solution, and acidify the aqueous phase to pH=3 with dilute hydrochloric acid. Combine the organic phases, dry over anhydrous sodium sulfate (Na₂SO₄), and remove the organic solvent by distillation under reduced pressure. The obtained crude product is purified by column chromatography, with a yield of 47%. ¹H-NMR (400 MHz, CD₃OD): δ=9.78 (s, 1H), 8.05 (s, 1H), 8.02 (d, J=7.3 Hz, 2H), 7.22 (d, J=7.3 Hz, 1H), 7.16 (s, 1H), 6.62 (s, 2H), 6.44-6.26 (dd, J=13.1 Hz, 2H), 3.83 (t, J=8.1 Hz, 4H), 3.68-3.55(m, 10H), 3.32 (t, J=5.1 Hz, 2H), 3.23 (s, 6H), 2.93 (t, J=8.1 Hz, 4H), 0.57 (s, 6H).

Synthesis of Molecular Rotor 19: Add Compound 58 (0.18 g, 0.27 mmol) and sodium acetate (0.11 g, 1.3 mmol) to 10 mL of acetic anhydride. After stirring at 120° C. for 45 minutes, remove the solvent by distillation under reduced pressure, extract with DCM, combine the organic phases, and dry over anhydrous sodium sulfate (Na₂SO₄). The obtained crude product is purified by column chromatography, with a yield of 81%. ¹H-NMR (400 MHz, CD₃OD): δ=8.10 (s, 1H), 8.05 (d, J=7.3 Hz, 2H), 7.31 (d, J=7.3 Hz, 1H), 7.27 (s, 1H), 6.69 (s, 2H), 6.25 (dd, J=13.1 Hz, 2H), 3.83 (t, J=8.1 Hz, 4H), 3.75-3.47 (m, 10H), 3.32 (t, J=5.1 Hz, 2H), 3.23 (s, 6H), 2.93 (t, J=8.1 Hz, 4H), 0.57 (s, 6H).

### Example 20

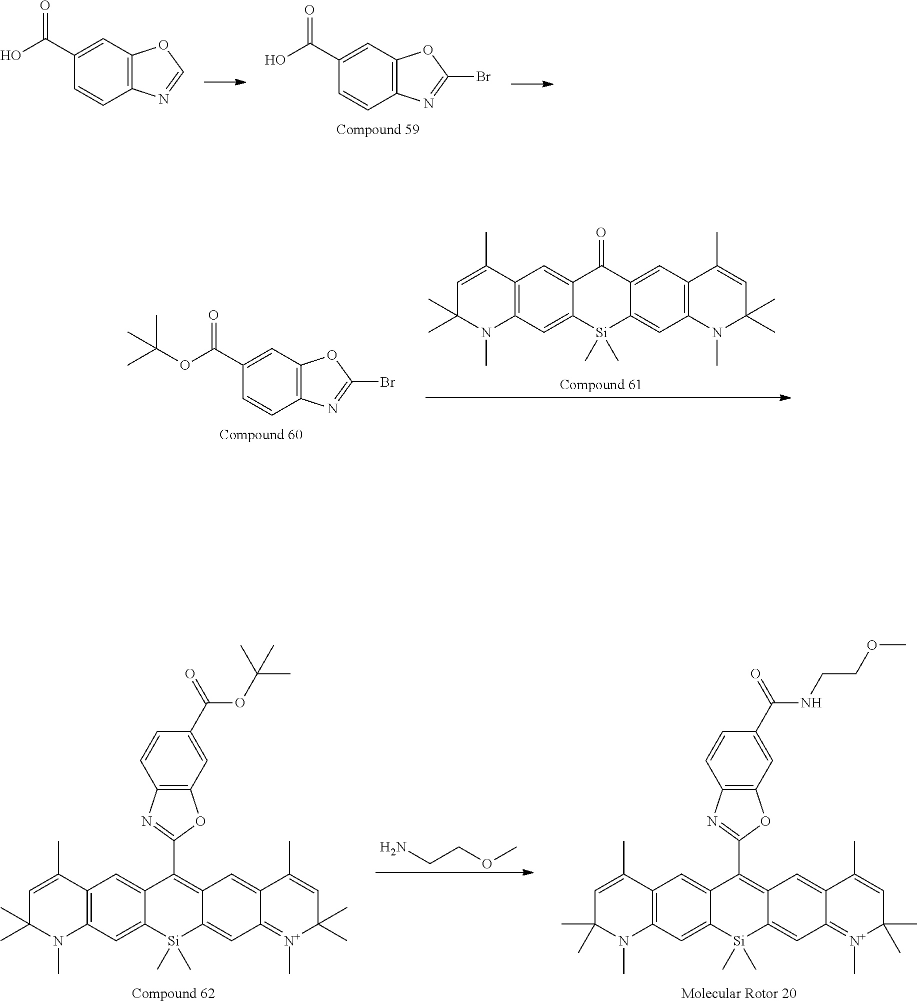

Synthesis of Compound 59: Refer to the synthesis method of Compound 9, with a yield of 92%. ¹H-NMR (400 MHz, CDCl₃): δ=13.02 (s, 1H), 8.30-8.28 (m, 1H), 8.10 (dd, J=8.4, 1.6 Hz, 1H), 7.89 (d, J=8.4 Hz, 1H).

Synthesis of Compound 60: Refer to the synthesis method of Compound 2, with a yield of 95%. ¹H-NMR (400 MHz, CDCl₃): δ=8.23-8.16 (m, 2H), 7.79 (d, J=1.8 Hz, 1H), 1.89 (s, 9H).

Synthesis of Compound 61: Refer to the literature J. Am. Chem. Soc. 2012, 134, 5029-5031 for synthesis, with a yield of 37%. ¹H-NMR (400 MHz, CD₃OD): δ=7.41 (s, 2H), 7.17 (s, 2H), 5.59 (s, 2H), 3.28 (s, 6H), 2.05 (s, 6H), 1.52 (s, 12H), 0.53 (s, 6H).

Synthesis of Compound 62: Refer to the synthesis method of Molecular Rotor 1, with a yield of 50%. ¹H-NMR (400 MHz, CD₃OD): δ=7.52-7.37 (m, 2H), 7.14 (d, J=7.3 Hz, 1H), 6.75 (s, 2H), 5.51 (s, 2H), 3.29 (s, 6H), 2.05 (s, 3H), 1.58 (s, 9H), 1.53 (s, 6H), 1.49 (s, 6H), 0.61 (s, 3H), 0.59 (s, 3H).

Synthesis of Molecular Rotor 20: Dissolve Compound 62 (50 mg, 0.078 mmol) in 8 mL of anhydrous DCM, then add 2 mL of trifluoroacetic acid. Stir at room temperature for 2 hours, and then remove the solvent by distillation under reduced pressure. Then dissolve the obtained solid in anhydrous DMF, add 2-methoxyethylamine (6.5 mg, 0.086 mmol), the condensing agent PyBop (61 mg, 0.12 mmol), and 0.1 mL of triethylamine, and stir at room temperature for 1 hour. After the reaction is completed, remove the solvent by distillation under reduced pressure, extract with DCM, combine the organic phases, dry over anhydrous sodium sulfate, remove the solvent by distillation under reduced pressure. After purification by column chromatography, the product can be obtained (31 mg, yield 62%). ¹H-NMR (400 MHz, CD₃OD): δ=7.38-7.23 (m, 2H), 7.04 (s, 2H), 7.00-6.96 (m, 1H), 6.59 (s, 2H), 3.72 (t, J=8.3 Hz, 4H), 3.67 (t, J=8.3 Hz, 2H), 3.53 (s, 3H) 3.13 (s, 6H), 3.07 (t, J=8.2 Hz, 2H), 2.04 (s, 3H), 1.59 (s, 9H), 1.54 (s, 6H), 1.38 (s, 6H), 0.48 (s, 3H), 0.45 (s, 3H).

### Example 21

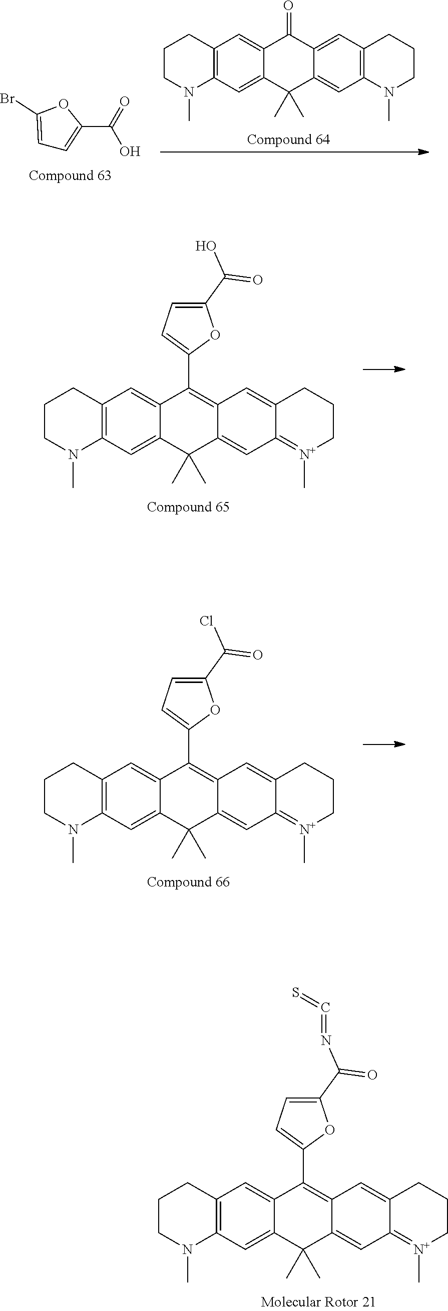

Synthesis of Compound 64: Refer to the literature J. Am. Chem. Soc. 2012, 134, 5029-5031 for synthesis, with a yield of 53%. ¹H-NMR (400 MHz, CD₃OD): δ=7.15 (s, 2H), 6.75 (s, 2H), 3.18 (t, J=5.9 Hz, 4H), 2.85 (s, 6H), 2.56 (t, J=6.6 Hz, 4H), 1.90 (dt, J=6.6, 5.9 Hz, 4H), 0.44 (s, 6H).

Synthesis of Compound 65: Refer to the synthesis method of Molecular Rotor 1, with a yield of 16%. ¹H-NMR (400 MHz, CD₃OD): δ=12.08 (s, 1H), 7.49 (s, 1H) 7.22 (s, 2H), 7.08 (d, J=7.3 Hz, 1H), 6.68 (s, 2H), 3.60 (t, J=5.1 Hz, 4H), 3.33 (s, 6H), 2.48 (t, J=5.9 Hz, 4H), 1.91 (dt, J=5.9, 5.1 Hz, 4H), 0.57 (s, 3H), 0.55 (s, 3H).

Synthesis of Compound 66: Refer to the synthesis method of Molecular Rotor 11, with a yield of 68%. ¹H-NMR (400 MHz, CD₃OD): δ=12.05 (s, 1H), 7.50 (s, 1H) 7.31 (s, 2H), 7.10 (d, J=7.3 Hz, 1H), 6.72 (s, 2H), 3.59 (t, J=5.1 Hz, 4H), 3.29 (s, 6H), 2.52 (t, J=5.9 Hz, 4H), 1.91 (dt, J=5.9, 5.1 Hz, 4H), 0.56 (s, 3H), 0.54 (s, 3H).

Synthesis of Molecular Rotor 21: Dissolve Compound 66 (80 mg, 0.17 mmol) in 20 mL of anhydrous tetrahydrofuran (THF), and add it dropwise to a pressure-resistant flask containing potassium thiocyanate (25 mg, 0.25 mmol). Heat the mixture under reflux at 60° C. for about 2 hours. After the reaction is completed, remove the solvent by rotary evaporation, extract with DCM, combine the organic phases, dry over anhydrous sodium sulfate, remove the solvent by distillation under reduced pressure. After purification by column chromatography, the product can be obtained (60 mg, yield 72%). ¹H-NMR (400 MHz, CD₃OD): δ=12.08 (s, 1H), 7.68 (s, 1H) 7.30 (s, 2H), 7.21 (d, J=7.3 Hz, 1H), 6.70 (s, 2H), 3.59 (t, J=5.1 Hz, 4H), 3.31 (s, 6H), 2.49 (t, J=5.9 Hz, 4H), 1.89 (dt, J=5.9, 5.1 Hz, 4H), 0.58 (s, 3H), 0.54 (s, 3H).

### Example 22

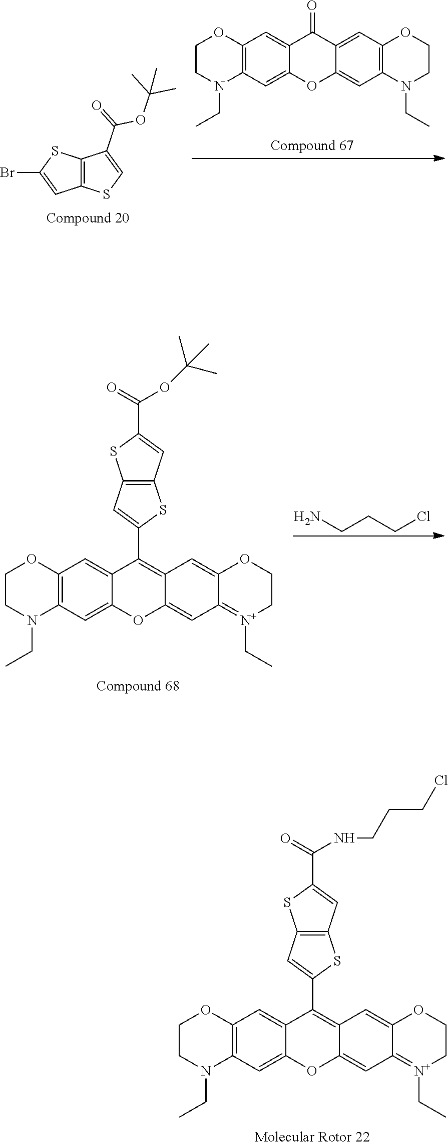

Synthesis of Compound 67: The compound was synthesized by referring to the method reported in J. Am. Chem. Soc. 2012, 134, 5029-5031, with a yield of 27%. ¹H-NMR (400 MHz, DMSO-D₆): 8=7.23 (d, J=6.56 Hz, 2H), 6.87 (s, 2H), 3.60 (m, 4H), 3.24 (m, 4H), 3.01 (m, 4H), 1.21 (t, J=6.99 Hz, 6H).

Synthesis of Compound 68: Referring to the synthesis method of Molecular Rotor 1, the compound was obtained with a yield of 36%. ¹H-NMR (400 MHz, DMSO-D₆): 8=8.23 (d, J=7.01 Hz, 1H), 7.91 (t, J=7.52 Hz, 1H), 7.82 (t, J=7.66 Hz, 1H), 7.48 (d, J=7.56 Hz, 1H), 6.78 (s, 2H), 3.66 (m, 4H), 3.29 (m, 4H), 3.06 (m, 4H), 1.56 (s, 9H), 1.24 (t, J=7.05 Hz, 6H).

Synthesis of Molecular Rotor 22: Dissolve Compound 68 (35 mg, 0.059 mmol) in 8 mL of anhydrous dichloromethane (DCM), then add 2 mL of trifluoroacetic acid. Stir the mixture at room temperature for 2 hours, and then remove the solvent by distillation under reduced pressure. Subsequently, dissolve the obtained solid in anhydrous N,N-dimethylformamide (DMF), and add 3-chloropropylamine (6 mg, 0.065 mmol), the condensing agent PyBop (46 mg, 0.089 mmol), and 0.1 mL of triethylamine. Stir the reaction mixture at room temperature for 1 hour. After the reaction is completed, remove the solvent by distillation under reduced pressure, extract the mixture with DCM, combine the organic phases, dry over anhydrous sodium sulfate, and remove the solvent by distillation under reduced pressure. The product can be obtained after purification by column chromatography (24 mg, yield 66%). ¹H-NMR (400 MHz, CD₃OD): δ=8.62 (s, 1H), 8.12 (s, 1H), 7.64 (m, 2H), 7.23 (d, J=6.5 Hz, 1H), 6.85 (s, 1H), 5.93 (s, 1H), 4.60 (m, 4H), 4.12 (m, 4H) 3.82 (m, 4H), 3.75 (m, 2H), 3.24 (t, J=6.6 Hz, 2H), 3.04 (t, J=6.5 Hz, 2H), 1.21 (t, J=6.99 Hz, 6H).

### Example 23

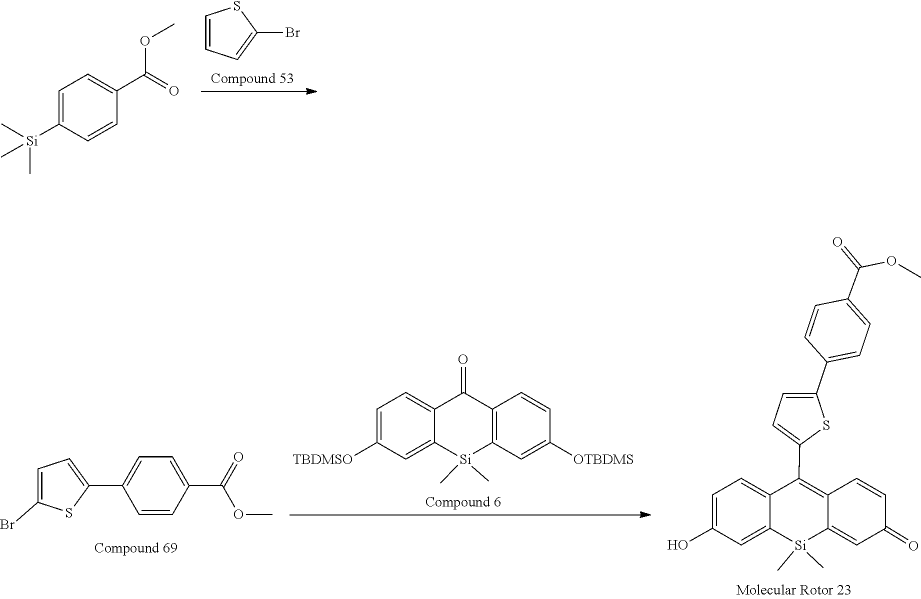

Synthesis of Compound 69: Refer to the literature J. Am. Chem. Soc. 2014, 136, 254-264 for synthesis, with a yield of 83%. ¹H-NMR (400 MHz, CDCl₃): δ=8.05 (d, J=8.5 Hz, 2H), 7.58 (d, J=8.5 Hz, 2H), 7.17 (d, J=3.9 Hz, 1H), 7.08 (d, J=3.9 Hz, 1H), 3.94 (s, 3H).

Synthesis of Molecular Rotor 23: Refer to the synthesis method of Molecular Rotor 2, with a yield of 62%. ¹H-NMR (400 MHz, CDCl₃): δ=7.94 (dt, J=7.8, 1.0 Hz, 1H), 7.80 (td, J=7.8, 1.0 Hz, 1H), 7.67 (td, J=7.8, 1.0 Hz, 2H), 7.38 (dt, J=7.8, 1.0 Hz, 2H), 7.23 (d, J=2.4 Hz, 2H), 6.83 (d, J=8.8 Hz, 2H), 6.76 (dd, J=8.8, 2.4 Hz, 2H), 3.92 (s, 3H), 0.64 (s, 3H), 0.56 (s, 3H).

### Example 24

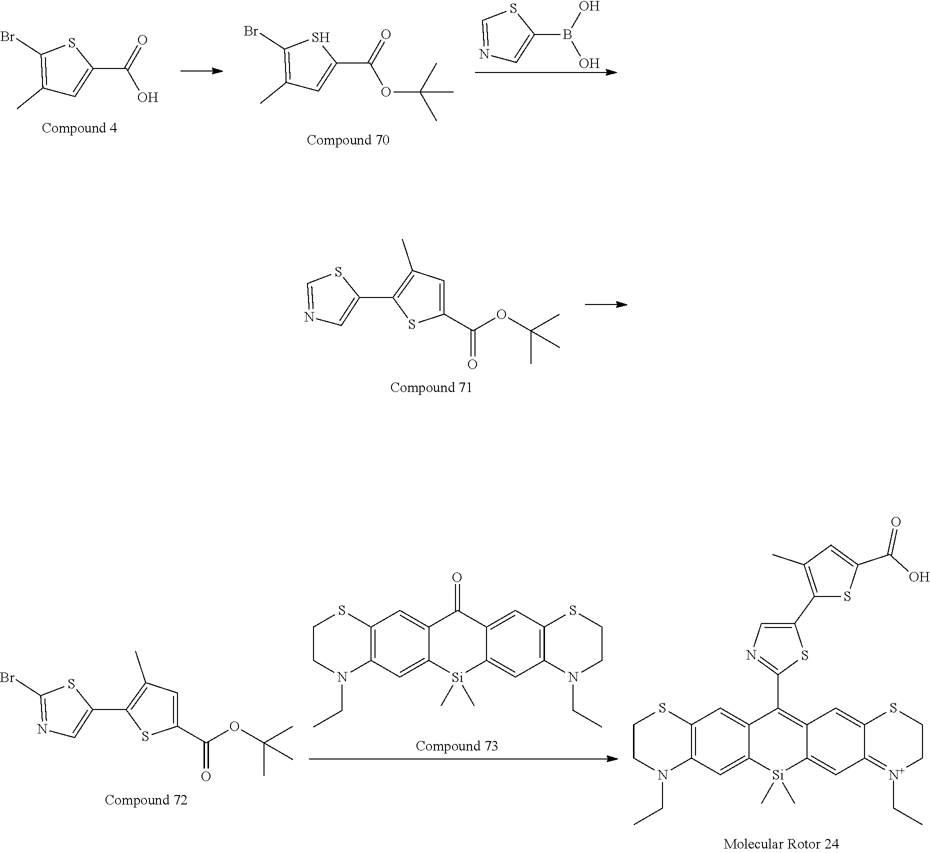

Synthesis of Compound 70: Refer to the synthesis method of Compound 2, with a yield of 85%. ¹H-NMR (400 MHz, CDCl₃): δ=7.54 (s, 1H), 2.57 (s, 3H), 1.56 (s, 9H).

Synthesis of Compound 71: Refer to the synthesis method of Compound 40, with a yield of 95%. ¹H-NMR (400 MHz, CDCl₃): δ=7.94 (s, 1H), 7.21 (d, J=3.5 Hz, 1H), 6.56 (d, J=3.4 Hz, 1H), 3.91 (s, 3H), 1.74 (s, 9H).

Synthesis of Compound 72: Refer to the synthesis method of Compound 9, with a yield of 89%. ¹H-NMR (400 MHz, CDCl₃): δ=7.82 (s, 1H), 7.76 (dd, J=8.3, 2.6 Hz, 1H), 2.56 (s, 3H), 1.75 (s, 9H).

Synthesis of Compound 73: Refer to the literature J. Am. Chem. Soc. 2012, 134, 5029-5031 for synthesis, with a yield of 20%. ¹H-NMR (400 MHz, CD₃OD): δ=7.15 (s, 2H), 7.02 (s, 2H), 3.60 (t, J=5.8 Hz, 4H), 3.48 (t, J=5.9 Hz, 4H), 3.18 (t, J=5.9 Hz, 4H), 2.85 (s, 6H), 0.60 (s, 6H).

Synthesis of Molecular Rotor 24: Refer to the synthesis method of Molecular Rotor 1. Then dissolve the obtained compound in 8 mL of anhydrous DCM, add 2 mL of trifluoroacetic acid, stir at room temperature for 2 hours, remove the solvent by distillation under reduced pressure, and purify by column chromatography to obtain the product, with a yield of 52%. ¹H-NMR (400 MHz, CD₃OD): δ=7.49-7.32 (m, 3H), 7.22 (s, 1H), 7.08 (d, J=7.3 Hz, 1H), 6.68 (s, 1H), 3.68 (t, J=5.9 Hz, 4H), 3.52 (t, J=5.9 Hz, 4H), 3.23 (t, J=5.9 Hz, 4H), 2.19 (s, 3H), 1.12 (s, 6H), 0.55 (s, 3H), 0.57 (s, 3H).

### Example 25

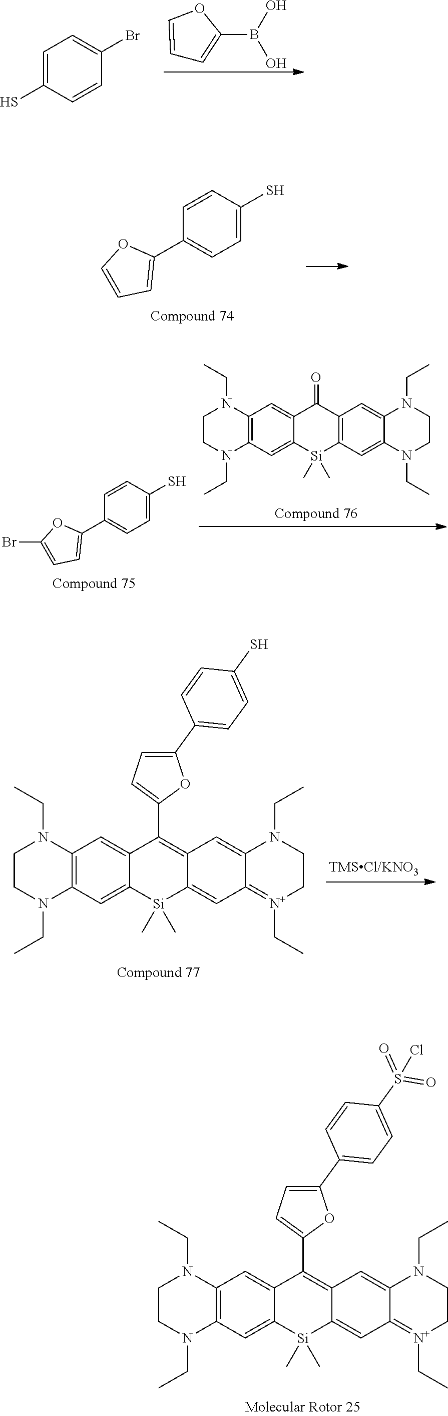

Synthesis of Compound 74: Refer to the synthesis method of Compound 40, with a yield of 92%. ¹H-NMR (400 MHz, CDCl₃): δ=7.68 (s, 1H), 7.49-7.32 (m, 4H), 7.01 (d, J=8.3 Hz, 1H), 6.82 (d, J=8.3 Hz, 1H).

Synthesis of Compound 75: Refer to the synthesis method of Compound 9, with a yield of 85%. ¹H-NMR (400 MHz, CDCl₃): δ=7.52-7.36 (m, 4H), 7.05 (d, J=8.1 Hz, 1H), 6.87 (d, J=8.2 Hz, 1H).

Synthesis of Compound 76: Refer to the literature Dyes and Pigments 99 (2013) 1132-1136 for synthesis, with a yield of 20%. ¹H-NMR (400 MHz, CD₃OD): δ=6.92 (s, 2H), 6.78 (s, 2H), 3.57 (s, 8H), 3.4 (t, J=5.9 Hz, 8H), 1.12 (t, J=6.9 Hz, 12H), 0.66 (s, 6H).

Synthesis of Compound 77: Refer to the synthesis method of Molecular Rotor 1, with a yield of 16%. ¹H-NMR (400 MHz, CD₃OD): δ=8.12 (s, 1H), 7.61-7.55 (m, 4H), 7.44 (m, 2H), 7.23 (d, J=6.5 Hz, 1H), 6.87 (s, 2H), 3.60 (m, 8H), 3.24 (m, 6H), 3.01 (m, 6H), 1.21 (t, J=6.8 Hz, 6H), 0.86 (t, J=6.7 Hz, 6H).

Synthesis of Molecular Rotor 25: Dissolve Compound 77 (45 mg, 0.072 mmol) in 20 mL of DCM, add trimethylsilyl chloride (TMS·Cl) (16.5 mg, 0.15 mmol) and potassium nitrate (KNO₃) (15.3 mg, 0.15 mmol) respectively, and then stir the reaction system at 30° C. After the reaction is completed, cool the mixture to room temperature, then filter to remove the residue. Extract the filtrate with water, combine the organic phases, dry over anhydrous sodium sulfate, remove the solvent by distillation under reduced pressure. The product can be obtained after purification by column chromatography (45 mg, yield 90%). ¹H-NMR (400 MHz, CD₃OD): δ=8.23 (s, 1H), 7.60-7.52 (m, 4H), 7.45 (m, 2H), 7.19 (d, J=6.6 Hz, 1H), 6.85 (s, 2H), 3.62 (m, 8H), 3.26 (m, 6H), 3.20 (m, 6H), 1.21 (t, J=6.9 Hz, 6H), 0.86 (t, J=6.2 Hz, 6H).

### Example 26

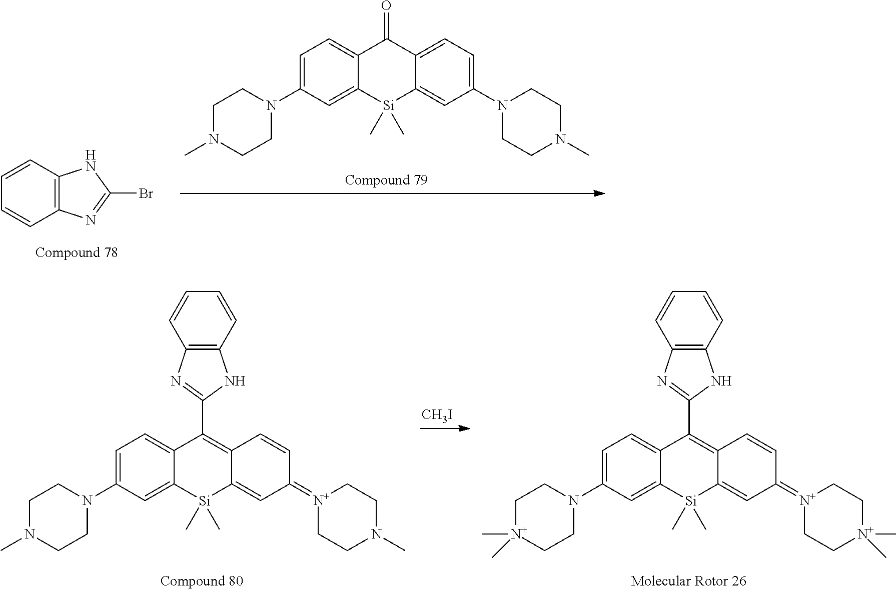

Synthesis of Compound 80: Refer to the synthesis method of Molecular Rotor 1, with a yield of 29%. ¹H-NMR (400 MHz, CD₃OD): δ=8.01 (d, J=7.3 Hz, 1H), 7.79-7.61 (m, 2H), 6.97 (d, J=1.6 Hz, 2H), 6.74-6.59 (m, 4H), 4.32 (t, J=7.3 Hz, 4H), 3.44 (t, J=5.3 Hz, 4H), 2.42 (m, J=6.9 Hz, 8H), 2.36 (s, 6H), 0.73 (t, J=5.5 Hz, 6H).

Synthesis of Molecular Rotor 26: Refer to the literature J. Am. Chem. Soc. 2019, 141, 14491-14495 for synthesis, with a yield of 62%. ¹H-NMR (400 MHz, CD₃OD): δ=8.00 (d, J=7.6 Hz, 1H), 7.79 (t, J=7.4 Hz, 1H), 7.72 (t, J=7.5 Hz, 1H), 7.19 (d, J=7.6 Hz, 1H), 6.87 (s, 2H), 6.80 (d, J=8.8 Hz, 2H), 6.62 (d, J=8.8 Hz, 1H), 3.61 (s, 8H), 3.53 (s, 8H), 3.18 (s, 12H), 0.73 (s, 6H).

### Example 27

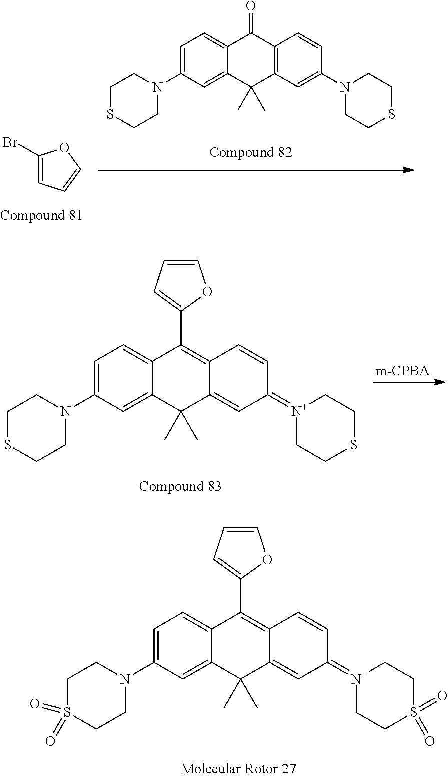 [SMILES: CC1(C)c2cc(N3CCSCC3)ccc2C(=O)c2ccc(N3CCSCC3)cc21 ; Brc1ccco1 ; CC1(C)C2=CC(=[N+]3CCSCC3)C=CC2=C(c2ccco2)c2ccc(N3CCSCC3)cc21 ; CC1(C)C2=CC(=[N+]3CCS(=O)(=O)CC3)C=CC2=C(c2ccco2)c2ccc(N3CCS(=O)(=O)CC3)cc21]

Synthesis of Compound 82: The compound was synthesized by referring to the method reported in Chem. Commun., 2020, 56, 715-718, with a yield of 74%. ¹H-NMR (400 MHz, CDCl₃): δ=8.26 (d, J=7.8 Hz, 2H), 6.95 (s, 2H), 6.91 (d, J=7.8 Hz, 2H), 3.80 (s, 8H), 2.76 (s, 8H), 1.69 (s, 6H).

Synthesis of Compound 83: Referring to the synthesis method of Molecular Rotor 1, the compound was obtained with a yield of 67%. ¹H-NMR (400 MHz, CD₃OD): δ=7.30 (d, J=1.8 Hz, 1H), 7.27 (d, J=7.8 Hz, 2H), 7.05 (d, J=9.0 Hz, 2H), 6.89 (dd, J=1.8, 7.8 Hz, 2H), 4.10 (t, J=4.8 Hz, 8H), 2.79 (t, J=4.8 Hz, 8H), 1.77 (s, 6H).

Synthesis of Molecular Rotor 27: Dissolve Compound 83 (61.3 mg, 0.1 mmol) in 20 mL of dichloromethane (DCM), and slowly add it dropwise at 0° C. to a solution of 10 mL DCM containing 3-chloroperoxybenzoic acid (138 mg, 0.81 mmol). Keep the reaction system stirred at 0° C. for 2 hours, and then cool it to room temperature and continue stirring for 4 hours. After the reaction is completed, wash the reaction solution successively with sodium bisulfite (NaHSO₃) and perchloric acid (HClO₄) solutions. Collect the organic phase, dry it over anhydrous sodium sulfate, remove the solvent by distillation under reduced pressure. The product can be obtained after purification by column chromatography (20 mg, yield 30%). ¹H-NMR (400 MHz, CD₃OD): δ=7.41 (d, J=2.4 Hz, 1H), 7.30 (d, J=7.8 Hz, 2H), 7.18 (d, J=9.6 Hz, 2H), 7.02-7.00 (dd, J=2.4 Hz, 2H), 4.23 (t, J=4.8 Hz, 8H), 3.23 (t, J=4.8 Hz, 8H), 1.81 (s, 6H).

### Example 28

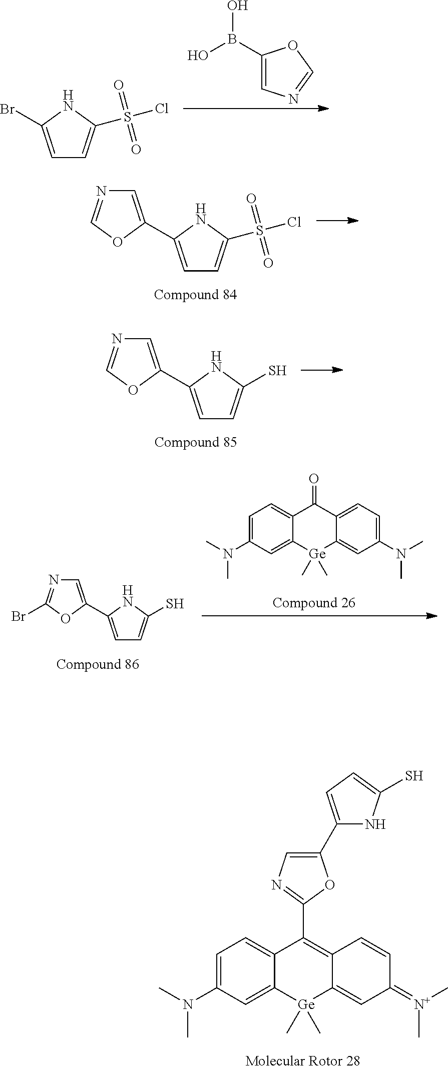

Synthesis of Compound 84: Refer to the synthesis method of Compound 40, with a yield of 90%. ¹H-NMR (400 MHz, DMSO-d₆): δ=11.94 (s, 1H), 7.90 (d, J=7.6 Hz, 1H), 7.09 (d, J=7.4 Hz, 1H), 6.48 (d, J=9.6 Hz, 2H).

Synthesis of Compound 85: Refer to the literature Res Chem Interned (2013) 39:4121-4125 for synthesis, with a yield of 96%. ¹H-NMR (400 MHz, DMSO-d₆): δ=12.46 (s, 1H), 11.94 (s, 1H), 7.90 (d, J=7.6 Hz, 1H), 7.11 (d, J=7.4 Hz, 1H), 6.45 (d, J=9.6 Hz, 2H).

Synthesis of Compound 86: Refer to the synthesis method of Compound 9, with a yield of 88%. ¹H-NMR (400 MHz, DMSO-d₆): δ=12.25 (s, 1H), 11.83 (s, 1H), 7.01 (d, J=7.4 Hz, 1H), 6.51 (d, J=9.4 Hz, 2H).

Synthesis of Molecular Rotor 28: Refer to the synthesis method of Molecular Rotor 1, with a yield of 68%. ¹H-NMR (400 MHz, CD₃OD): δ=12.02 (s, 1H), 11.71 (s, 1H), 7.85 (d, J=7.4 Hz, 2H), 7.54 (dd, J=7.6, 2.2 Hz, 2H), 7.06 (d, J=2.8 Hz, 2H), 6.62 (d, J=9.4 Hz, 2H), 6.53 (dd, J=9.2 Hz, 1H), 3.06 (s, 12H), 0.68 (s, 6H).

### Example 29

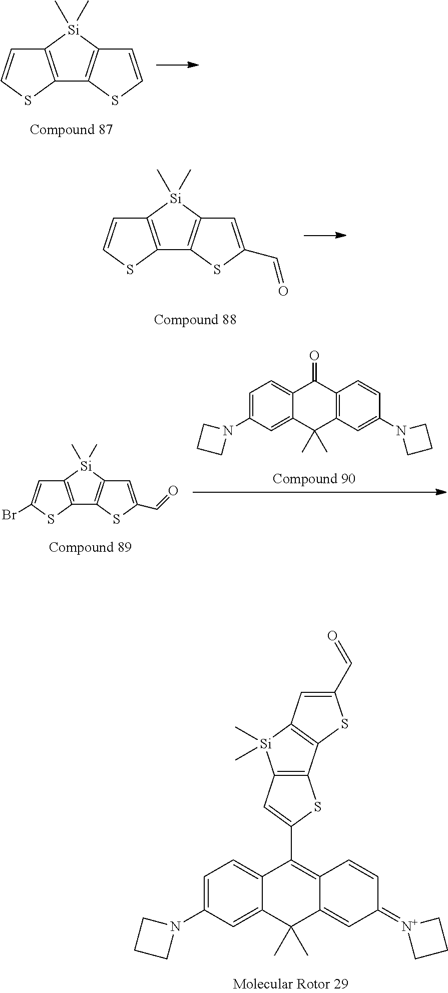 [SMILES: C[Si]1(C)c2ccsc2-c2sccc21 ; C[Si]1(C)c2ccsc2-c2sc(C=O)cc21 ; C[Si]1(C)c2cc(Br)sc2-c2sc(C=O)cc21 ; CC1(C)c2cc(N3CCC3)ccc2C(=O)c2ccc(N3CCC3)cc21 ; CC1(C)C2=CC(=[N+]3CCC3)C=CC2=C(c2cc3c(s2)-c2sc(C=O)cc2[Si]3(C)C)c2ccc(N3CCC3)cc21]

Synthesis of Compound 87: Refer to the literature ACS Appl. Energy Mater. 2019, 2,5600-5612 for synthesis, with a yield of 75%. ¹H-NMR (400 MHz, CDCl₃): δ=7.32 (d, J=4.8 Hz, 2H), 7.20 (d, J=4.8 Hz, 2H), 0.55 (s, 6H).

Synthesis of Compound 88: Refer to the literature ACS Appl. Energy Mater 2019, 2, 5600-5612 for synthesis, with a yield of 69%. ¹H-NMR (400 MHz, CDCl₃): δ=9.88 (s, 1H), 7.74 (s, 1H), 7.42 (d, J=4.8 Hz, 1H), 7.15 (d, J=4.8 Hz, 1H), 0.47 (s, 6H).

Synthesis of Compound 89: Refer to the synthesis method of Compound 9, with a yield of 93%. ¹H-NMR (400 MHz, CDCl₃): δ=9.86 (s, 1H), 7.73 (s, 1H), 7.11 (s, 1H), 0.46 (s, 6H).

Synthesis of Compound 90: Refer to the literature Nat Methods 17, 815-821 (2020) for synthesis, with a yield of 43%. ¹H-NMR (400 MHz, CD₃OD): δ=7.69-7.59 (m, 2H), 7.33 (d, J=9.2 Hz, 2H), 7.21-7.16 (m, 2H), 4.27-4.13 (m, 8H), 3.94 (m, 4H), 1.51 (s, 6H).

Synthesis of Molecular Rotor 29: Refer to the synthesis method of Molecular Rotor 1, with a yield of 76%. ¹H-NMR (400 MHz, CD₃OD): δ=10.41 (d, J=8.1 Hz, 1H), 8.37 (dd, J=8.2, 1.6 Hz, 1H), 7.89 (d, J=1.6 Hz, 1H), 7.18 (d, J=9.2 Hz, 2H), 6.70 (dd, J=9.2, 2.0 Hz, 2H), 6.45 (d, J=2.0 Hz, 2H), 4.30-4.21 (m, 8H), 4.08 (m, 4H), 1.54 (s, 6H).

### Example 30

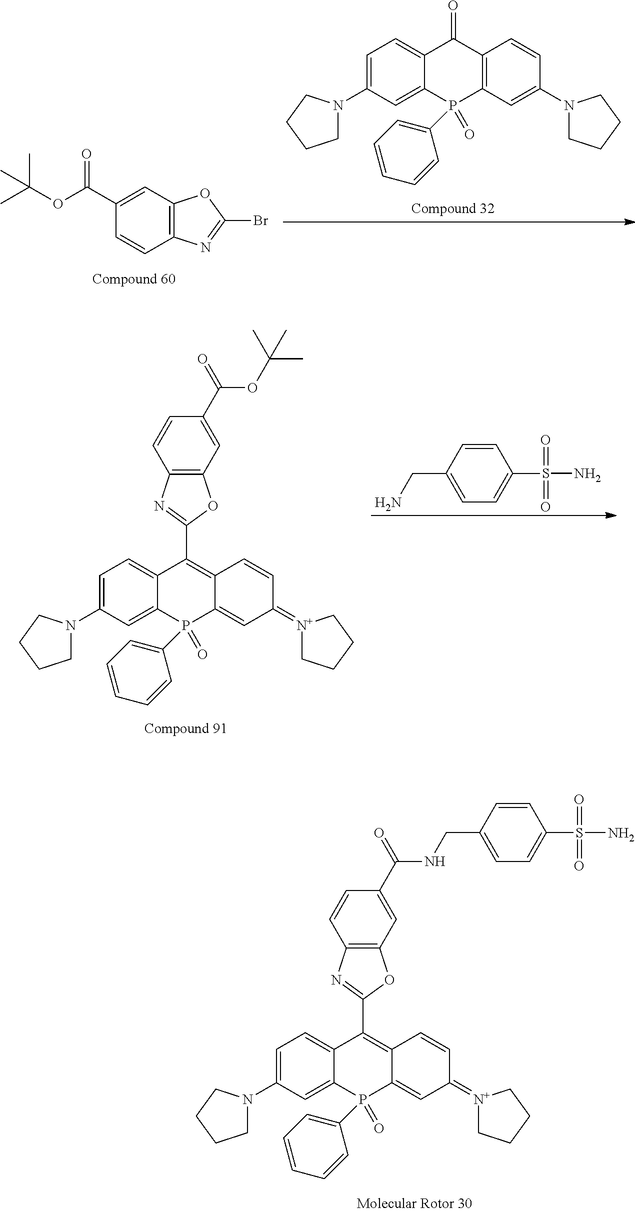 [SMILES: CC(C)(C)OC(=O)c1ccc2nc(Br)oc2c1 ; O=C1c2ccc(N3CCCC3)cc2P(=O)(c2ccccc2)c2cc(N3CCCC3)ccc21 ; CC(C)(C)OC(=O)c1ccc2nc(C3=C4C=CC(=[N+]5CCCC5)C=C4P(=O)(c4ccccc4)c4cc(N5CCCC5)ccc43)oc2c1 ; NCc1ccc(S(N)(=O)=O)cc1 ; NS(=O)(=O)c1ccc(CNC(=O)c2ccc3nc(C4=C5C=CC(=[N+]6CCCC6)C=C5P(=O)(c5ccccc5)c5cc(N6CCCC6)ccc54)oc3c2)cc1]

Synthesis of Compound 91: Refer to the synthesis method of Molecular Rotor 1, with a yield of 66%. ¹H-NMR (400 MHz, CD₃OD): δ=7.75-7.67 (m, 2H), 7.53-7.46 (m, 5H), 7.39 (s, 1H), 6.78 (dd, J=13.8, 2.6 Hz, 2H), 6.77 (d, J=8.8, 5.9 Hz, 2H), 6.45 (dd, J=8.8, 2.5 Hz, 2H), 3.71 (t, J=7.4 Hz, 8H), 2.65 (t, J=7.3 Hz, 8H), 1.46 (s, 9H).

Synthesis of Molecular Rotor 30: Dissolve Compound 91 (50 mg, 0.078 mmol) in 8 mL of anhydrous dichloromethane (DCM), then add 2 mL of trifluoroacetic acid. Stir at room temperature for 2 hours, and then remove the solvent by distillation under reduced pressure. Subsequently, dissolve the obtained solid in anhydrous N,N-dimethylformamide (DMF), add sulfanilamide (16 mg, 0.085 mmol), the condensing agent PyBop (61 mg, 0.12 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (48 mg, yield 82%). ¹H-NMR (400 MHz, CD₃OD): δ=7.77 (m, 2H), 7.71 (m, 5H), 7.44 (m, 5H), 7.24 (m, 2H), 6.86 (m, 2H), 6.20 (m, 1H), 5.89 (m, 1H), 4.35 (s, 2H), 3.75 (t, J=7.4 Hz, 8H), 2.56 (t, J=7.4 Hz, 8H).

### Example 31

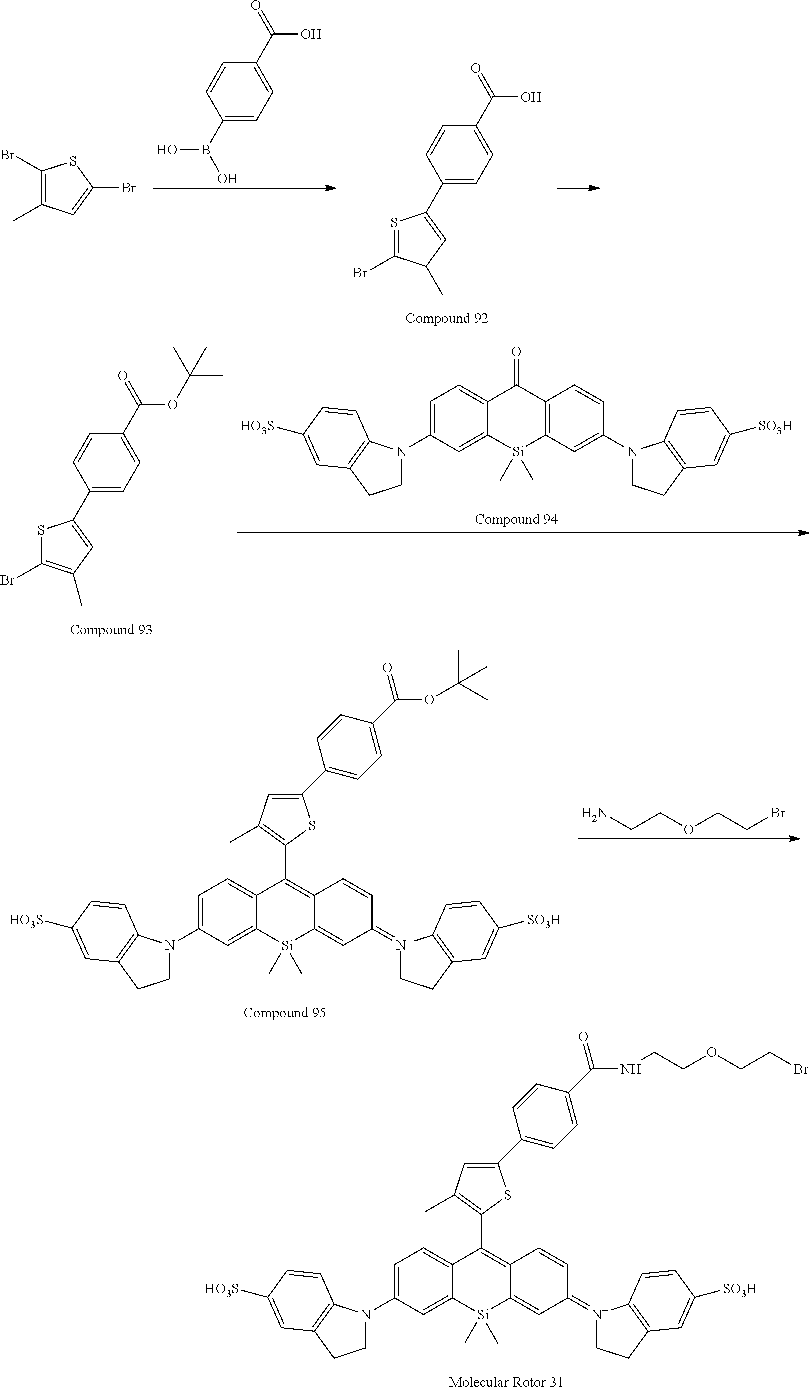

Synthesis of Compound 92: Refer to the synthesis method of Compound 40, with a yield of 92%. ¹H-NMR (400 MHz, CDCl₃): δ=7.72-7.65 (m, 2H), 7.47-7.41 (m, 1H), 7.36-7.33 (m, 1H), 6.84 (m, 1H), 2.64 (s, 3H).

Synthesis of Compound 93: Refer to the synthesis method of Compound 2, with a yield of 65%. ¹H-NMR (400 MHz, CDCl₃): δ=7.69 (m, 2H), 7.44 (m, 1H), 7.40 (m, 1H), 6.84 (m, 1H), 2.64 (s, 3H), 1.76 (m, 9H).

Synthesis of Compound 94: Refer to the literature J. Am. Chem. Soc. 2015, 137, 4759-4765 for synthesis, with a yield of 74%. ¹H-NMR (400 MHz, DMSO-d₆): δ=8.37 (d, J=8.7 Hz, 2H), 7.66-7.63 (m, 4H), 7.49 (s, 2H), 7.45 (dd, J=2.4, 8.7 Hz, 2H), 7.29 (d, J=8.7 Hz, 2H), 4.15 (t, J=9.0 Hz, 4H), 3.22 (t, J=9.0 Hz, 4H), 0.54 (s, 6H).

Synthesis of Compound 95: Refer to the synthesis method of Molecular Rotor 1, with a yield of 76%. ¹H-NMR (400 MHz, DMSO-d₆): δ=7.82 (d, J=2.10 Hz, 2H), 7.59-7.45 (m, 11H), 7.27 (d, J=7.50 Hz, 1H), 7.15 (d, J=9.10 Hz, 2H), 4.41 (t, J=7.50 Hz, 4H), 3.25 (t, J=7.50 Hz, 4H), 2.05 (s, 3H), 1.45 (s, 9H), 0.70 (s, 3H), 0.67 (s, 3H).

Synthesis of Molecular Rotor 31: Dissolve Compound 95 (70 mg, 0.078 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add 2,2′-oxybis(ethylamine) bromide (9 mg, 0.087 mmol), the condensing agent PyBop (61 mg, 0.12 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (66 mg, yield 85%). ¹H-NMR (400 MHz, DMSO-d₆): δ=7.84-7.75 (m, 6H), 7.62 (d, J=8.7 Hz, 2H), 7.43-7.27 (m, 7H), 6.68 (m, 2H), 4.43 (t, J=6.6 Hz, 4H), 4.13 (t, J=6.1 Hz, 4H), 3.87 (t, J=5.6 Hz, 4H), 3.33 (m, 4H), 2.05 (s, 3H), 0.70 (s, 6H).

### Example 32

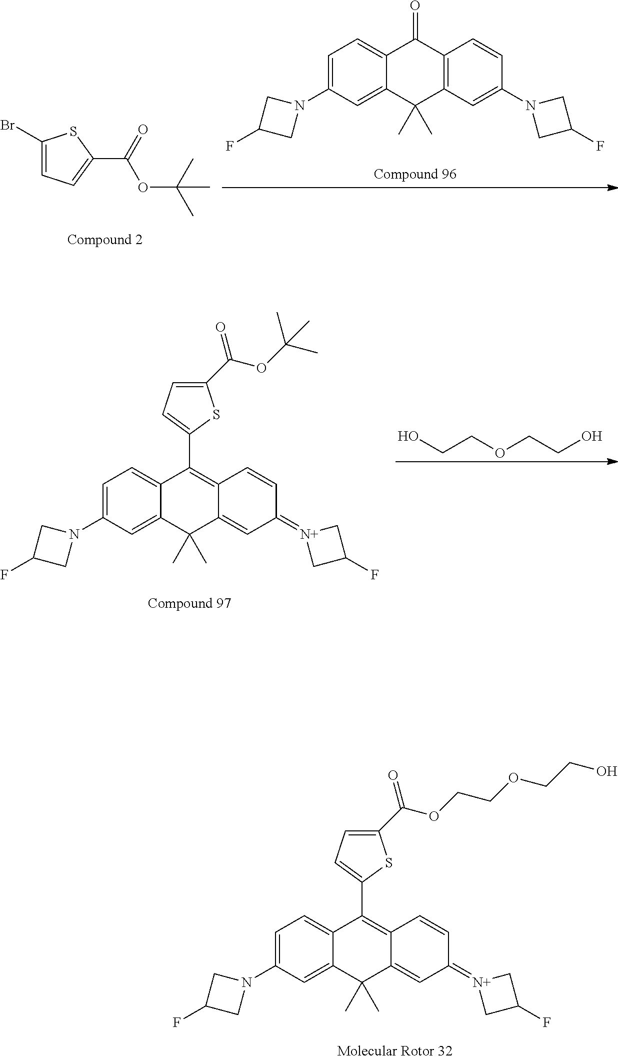

Synthesis of Compound 96: Refer to the literature Nat Methods 17, 815-821 (2020) for synthesis, with a yield of 43%. ¹H-NMR (400 MHz, CD₃OD): δ=7.27 (d, J=9.2 Hz, 2H), 6.78 (dd, J=9.2, 2.0 Hz, 2H), 6.61 (d, J=2.0 Hz, 2H), 5.55 (dd, J=6.0, 3.1 Hz, 2H), 4.61-4.48 (m, 4H), 4.39-4.23 (m, 4H), 4.65-4.56 (m, 8H), 4.15 (s, 2H), 1.70 (s, 6H).

Synthesis of Compound 97: Refer to the synthesis method of Molecular Rotor 1, with a yield of 69%. ¹H-NMR (400 MHz, CD₃OD): δ=8.19-8.13 (m, 1H), 7.69 (td, J=7.6, 1.5 Hz, 1H), 7.64 (td, J=7.4, 1.6 Hz, 1H), 7.49 (d, J=9.2 Hz, 2H), 7.23-7.18 (m, 1H), 6.85 (dd, J=9.2, 2.0 Hz, 2H), 1.55 (s, 9H), 1.70 (s, 6H).

Synthesis of Molecular Rotor 32: Dissolve Compound 97 (75 mg, 0.14 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add diethylene glycol (16 mg, 0.15 mmol), 4-dimethylaminopyridine (DMAP) (26 mg, 0.21 mmol), and 1-ethyl-3-(3-dimethylaminopropyl)carbodiimide (EDC) (40 mg, 0.21 mmol). Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (63 mg, yield 79%). ¹H-NMR (400 MHz, CD₃OD): δ=7.43 (d, J=9.1 Hz, 2H), 6.76 (dd, J=9.2, 2.2 Hz, 2H), 6.62 (d, J=2.2 Hz, 2H), 5.59 (dd, J=6.0, 3.0 Hz, 2H), 4.70-4.56 (m, 4H), 4.47-4.34 (m, 4H), 3.69-3.57 (m, 8H), 1.70 (s, 6H).

### Example 33

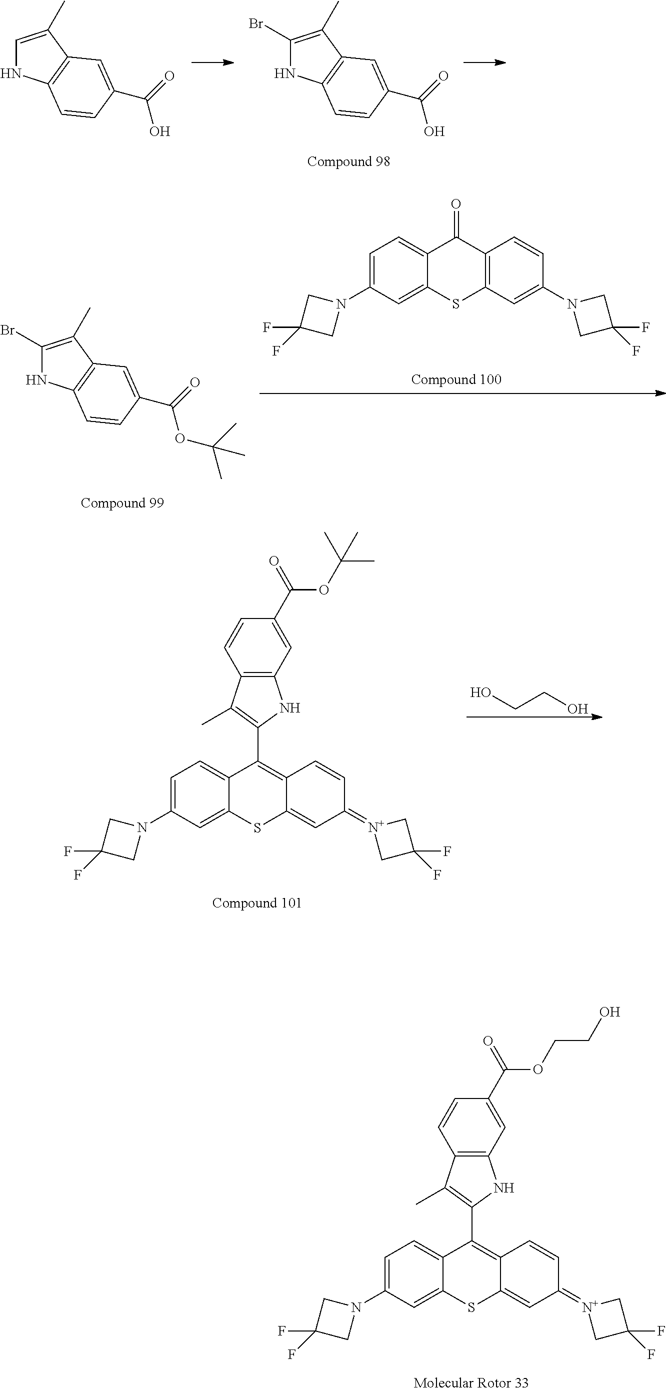

Synthesis of Compound 98: Refer to the synthesis method of Compound 9, with a yield of 92%. ¹H-NMR (400 MHz, CDCl₃): δ=8.29 (d, J=8.7 Hz, 1H), 7.61 (d, J=1.9 Hz, 1H), 7.43 (dd, J=8.7, 1.9 Hz, 1H), 7.16 (s, 1H), 2.57 (s, 3H).

Synthesis of Compound 99: Refer to the synthesis method of Compound 2, with a yield of 95%. ¹H-NMR (400 MHz, CDCl₃): δ=8.43 (d, J=7.2 Hz, 1H), 7.52-7.48 (m, 1H), 7.37 (td, J=7.3, 1.5 Hz, 1H), 7.30 (td, J=7.3, 1.5 Hz, 1H), 2.62 (s, 3H), 1.62 (s, 9H).

Synthesis of Compound 100: Refer to the literature Nat Methods 17, 815-821 (2020) for synthesis, with a yield of 23%. ¹H-NMR (400 MHz, CD₃OD): δ=7.40 (d, J=9.3 Hz, 2H), 6.83 (d, J=2.3 Hz, 2H), 6.68 (dd, J=9.4, 2.3 Hz, 2H), 4.35-4.22 (m, 8H).

Synthesis of Compound 101: Refer to the synthesis method of Molecular Rotor 1, with a yield of 73%. ¹H-NMR (400 MHz, CD₃OD): δ=7.37 (dd, J=9.1, 0.5 Hz, 2H), 6.72 (dd, J=9.2, 2.2 Hz, 2H), 6.58 (d, J=2.2 Hz, 3H), 6.26 (dd, J=6.0, 3.0 Hz, 2H), 4.66-4.53 (m, 4H), 4.44-4.30 (m, 4H), 2.37 (s, 3H), 1.62 (s, 9H).

Synthesis of Molecular Rotor 33: Dissolve Compound 101 (60 mg, 0.098 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add ethylene glycol (6.8 mg, 0.11 mmol), 4-dimethylaminopyridine (DMAP) (18 mg, 0.15 mmol), and 1-ethyl-3-(3-dimethylaminopropyl)carbodiimide (EDC) (28 mg, 0.15 mmol). Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (48 mg, yield 82%). ¹H-NMR (400 MHz, CD₃OD): δ=6.75 (d, J=8.6 Hz, 2H), 6.26 (d, J=2.3 Hz, 3H), 6.22 (dd, J=8.6, 2.3 Hz, 2H), 6.05 (dd, J=6.1, 3.5 Hz, 2H), 4.31-4.19 (m, 4H), 4.12-4.00 (m, 4H), 3.84 (t, J=8.3 Hz, 2H), 3.72 (t, J=8.1 Hz, 2H), 2.37 (s, 3H).

### Example 34

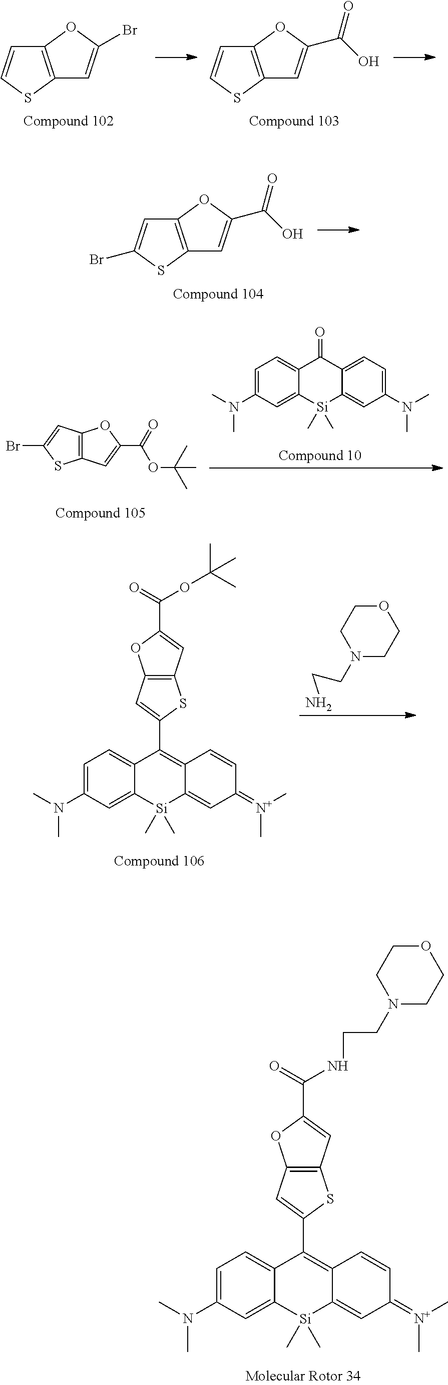

Synthesis of Compound 102: The compound was synthesized by referring to the method reported in Org. Lett. 2009, 11, 14, 3144-3147. ¹H-NMR (400 MHz, CDCl₃): δ=7.57 (d, J=2.1 Hz, 1H), 7.13 (d, J=0.5 Hz, 1H), 6.66 (dd, J=2.1, 0.5 Hz, 1H).

Synthesis of Compound 103: The compound was synthesized by referring to the method reported in Angew. Chem. Int. Ed. 2018, 57, 6910. ¹H-NMR (400 MHz, CDCl₃): δ=7.19 (d, J=5.3 Hz, 1H), 7.03 (dd, J=5.3, 0.5 Hz, 1H), 6.69 (d, J=0.5 Hz, 1H).

Synthesis of Compound 104: Refer to the synthesis method of Compound 9, with a yield of 60%. ¹H-NMR (400 MHz, CDCl₃): δ=7.12 (d, J=5.1 Hz, 1H), 6.96 (dd, J=5.1, 0.4 Hz, 1H).

Synthesis of Compound 105: Refer to the synthesis method of Compound 2, with a yield of 56%. ¹H-NMR (400 MHz, CDCl₃): δ=7.16 (d, J=5.3 Hz, 1H), 6.91 (dd, J=5.2, 0.4 Hz, 1H), 1.42 (s, 9H).

Synthesis of Compound 106: Refer to the synthesis method of Molecular Rotor 1, with a yield of 65%. ¹H-NMR (400 MHz, CD₃OD): δ=7.68 (d, J=6.9 Hz, 1H), 7.29 (m, 2H), 7.10 (d, J=7.5 Hz, 1H), 7.02 (d, J=6.6 Hz, 1H), 6.78 (d, J=8.3 Hz, 1H), 6.68 (d, J=2.5 Hz, 1H), 6.43 (d, J=2.6 Hz, 1H), 3.09 (s, 12H), 1.56 (s, 9H), 1.38 (s, 6H).

Synthesis of Molecular Rotor 34: Dissolve Compound 106 (85 mg, 0.17 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add N-(2-aminoethyl)morpholine (24 mg, 0.19 mmol), the condensing agent PyBop (131 mg, 0.25 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (85 mg, yield 39%). ¹H-NMR (400 MHz, CD₃OD): δ=7.16-7.10 (m, 3H), 6.88 (t, J=7.5 Hz, 1H), 6.81 (t, J=7.5 Hz, 1H), 6.47 (d, J=7.5 Hz, 2H), 6.08 (d, J=9.5 Hz, 1H), 3.70 (q, J=7.2 Hz, 8H), 3.52 (t, J=7.0 Hz, 2H), 3.36 (t, J=5.6 Hz, 2H), 1.54 (t, J=7.2 Hz, 12H), 0.62 (s, 3H), 0.61 (s, 3H).

### Example 35

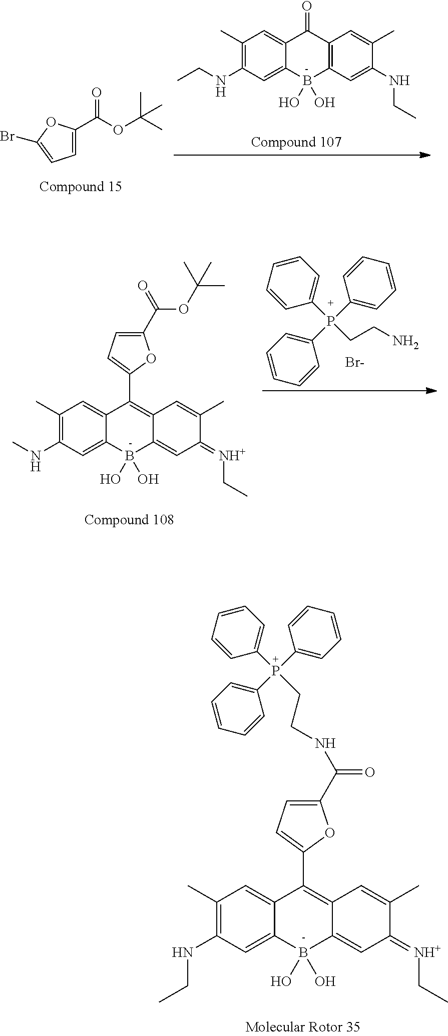

Synthesis of Compound 107: Refer to the literature Angew. Chem. Int. Ed., 2017, 56, 4197-4200 for synthesis, with a yield of 12%. ¹H-NMR (400 MHz, CD₃OD): δ=7.39 (d, J=2.5 Hz, 2H), 6.74 (d, J=8.7 Hz, 2H), 6.40 (dd, J=8.7, 2.5 Hz, 2H), 4.01 (d, J=7.4 Hz, 2H), 3.42 (m, 4H), 2.36 (s, 6H), 1.46 (t, J=7.3 Hz, 6H).

Synthesis of Compound 108: Refer to the synthesis method of Molecular Rotor 1, with a yield of 66%. ¹H-NMR (400 MHz, CD₃OD): δ=8.77 (t, J=5.2 Hz, 1H), 8.39 (d, J=8.3 Hz, 1H), 8.19 (dd, J=8.2, 1.8 Hz, 1H), 7.75 (d, J=1.8 Hz, 1H), 7.19 (d, J=9.2 Hz, 2H), 6.69 (dd, J=9.3, 2.0 Hz, 2H), 4.47 (d, J=2.0 Hz, 2H), 3.54 (m, 4H), 2.36 (s, 6H), 1.76 (s, 9H), 1.56 (t, J=7.3 Hz, 6H).

Synthesis of Molecular Rotor 35: Dissolve Compound 108 (75 mg, 0.15 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add (2-aminoethyl)triphenylphosphonium bromide (65 mg, 0.17 mmol), the condensing agent PyBop (120 mg, 0.23 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (96 mg, yield 87%). ¹H-NMR (400 MHz, CD₃OD): δ=7.44 (t, J=5.2 Hz, 1H), 7.36 (m, 16H), 7.06 (d, J=8.3 Hz, 1H), 6.74 (dd, J=8.2, 1.8 Hz, 1H), 6.09 (d, J=1.8 Hz, 1H), 6.50 (d, J=9.2 Hz, 2H), 6.20 (dd, J=9.3, 2.0 Hz, 1H), 4.07 (d, J=2.0 Hz, 2H), 3.45 (m, 4H), 3.03 (t, J=6.2 Hz, 2H), 2.36 (s, 6H), 1.56 (t, J=7.3 Hz, 6H).

### Example 36

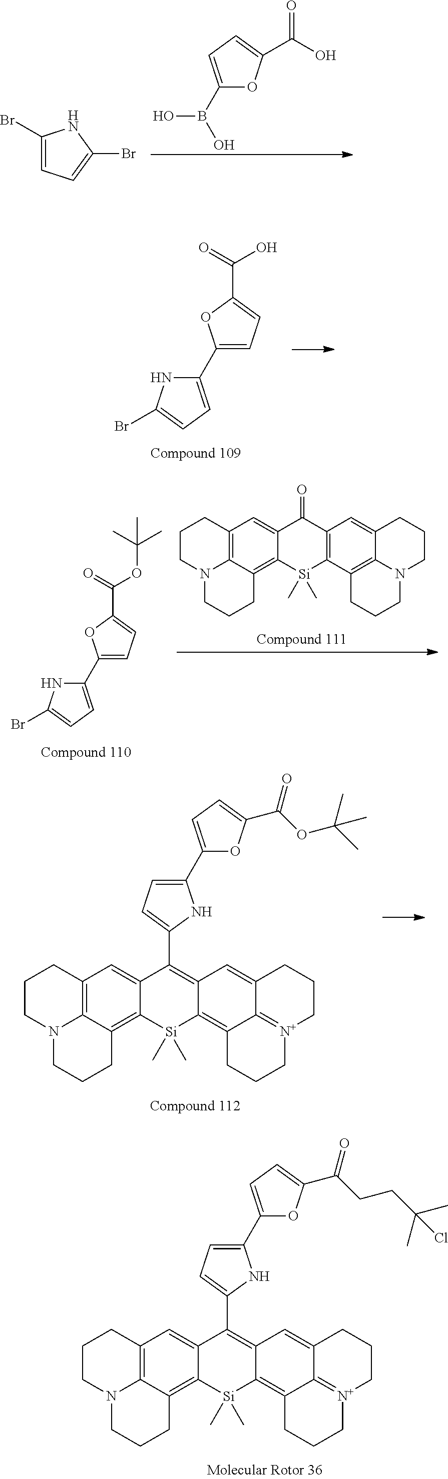

Synthesis of Compound 109: Refer to the synthesis method of Compound 40, with a yield of 76%. ¹H-NMR (400 MHz, CDCl₃): δ=7.49 (s, 1H), 7.02 (d, J=1.5 Hz, 1H), 6.24 (m, 2H).

Synthesis of Compound 110: Refer to the synthesis method of Compound 2, with a yield of 82%. ¹H-NMR (400 MHz, CDCl₃): δ=7.64 (dd, J=8.2 Hz, 1H), 6.94 (dt, J=8.0, 1.6 Hz, 1H), 6.85 (dt, J=7.1 Hz, 1H), 6.78 (m, 1H), 1.64 (s, 9H).

Synthesis of Compound 111: Refer to the literature Chem. Sci., 2019, 10, 9028-9037 for synthesis, with a yield of 20%. ¹H-NMR (400 MHz, CDCl₃): δ=8.07 (s, 2H), 3.29-3.27 (m, 8H), 2.91-2.80 (m, 8H), 2.03-1.96 (m, 8H), 0.59 (s, 6H).

Synthesis of Compound 112: Refer to the synthesis method of Molecular Rotor 1, with a yield of 66%. ¹H-NMR (400 MHz, CDCl₃): δ=7.81 (d, J=8.0 Hz, 2H), 7.38 (d, J=8 Hz, 2H), 6.93 (s, 2H), 4.72 (s, 2H), 3.77-3.75 (m, 4H), 3.64 (m, 4H), 3.39 (s, 4H), 2.95-2.90 (m, 4H), 2.75 (m, 4H), 1.34 (s, 9H), 0.68 (s, 3H), 0.61 (s, 3H).

Synthesis of Molecular Rotor 36: Refer to the literature Nat Commun 12, 4065 (2021) for synthesis, with a yield of 70%. ¹H-NMR (400 MHz, CDCl₃): δ=7.76 (t, J=5.3 Hz, 1H), 7.43 (d, J=8.2 Hz, 1H), 7.22 (dd, J=8.2, 1.8 Hz, 1H), 6.79 (d, J=1.8 Hz, 1H), 6.36 (d, J=9.2 Hz, 2H), 3.35-3.30 (m, 8H), 3.18-3.12 (m, 2H), 2.85-2.82 (m, 8H), 2.15-2.08 (m, 8H), 1.55 (d, J=0.7 Hz, 6H), 0.56 (s, 6H).

### Example 37

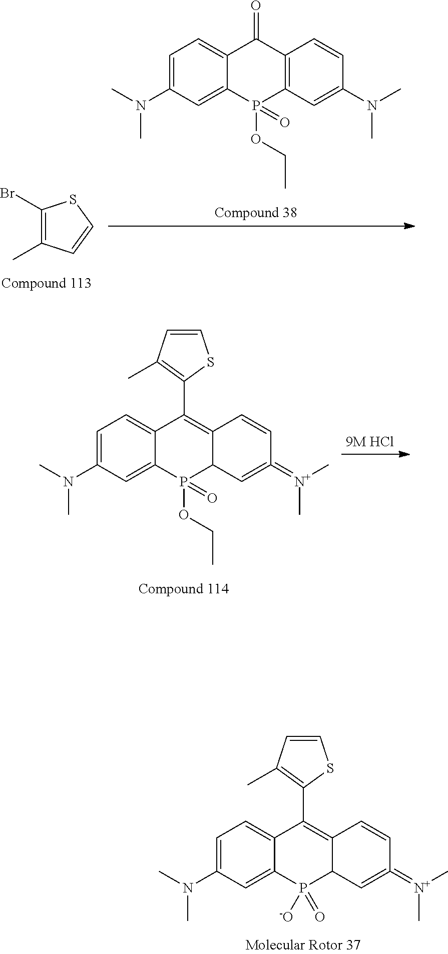

Synthesis of Compound 114: Refer to the synthesis method of Molecular Rotor 1, with a yield of 14%. ¹H-NMR (400 MHz, CD₃OD): δ=7.65-7.37 (m, 2H), 7.18 (t, J=7.8 Hz, 1H), 7.04 (td, J=10.3, 6.8 Hz, 3H), 6.83 (dd, J=9.7, 2.8 Hz, 2H), 4.09 (dp, J=9.6, 7.2 Hz, 2H), 3.38 (s, 12H), 2.06 (d, J=21.9 Hz, 3H), 1.24 (dt, J=18.1, 7.0 Hz, 3H).

Synthesis of Molecular Rotor 37: Refer to the synthesis method of Molecular Rotor 13, with a yield of 14%. ¹H-NMR (400 MHz, CD₃OD): δ=7.46-7.29 (m, 3H), 7.08 (d, J=7.5 Hz, 1H), 6.43 (d, J=6.1 Hz, 3H), 3.12 (s, 12H), 2.40 (s, 3H).

### Example 38

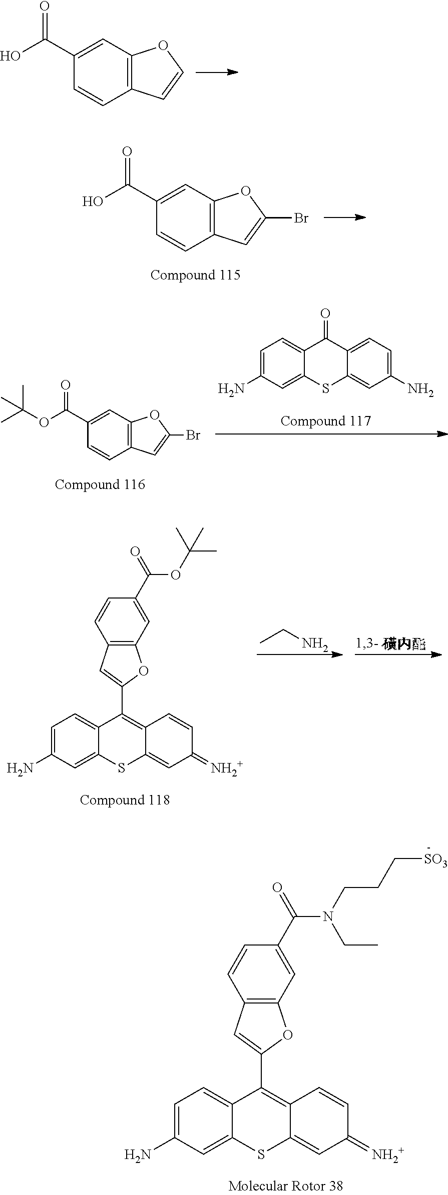

Synthesis of Compound 115: Refer to the synthesis method of Compound 9, with a yield of 90%. ¹H-NMR (400 MHz, DMSO-d₆): δ=12.90 (s, 1H), 8.13 (d, J=1.9 Hz, 2H), 7.73 (s, 1H), 7.16 (s, 1H).

Synthesis of Compound 116: Refer to the synthesis method of Compound 2, with a yield of 96%. ¹H-NMR (400 MHz, DMSO-d₆): δ=8.15 (d, J=1.9 Hz, 2H), 7.65 (s, 1H), 7.21 (s, 1H), 1.65 (s, 9H).

Synthesis of Compound 117: Refer to the literature J. Med. Chem. 2009, 52, 10, 3328-3341 for synthesis, with a yield of 40%. ¹H-NMR (400 MHz, CDCl₃): δ=7.75 (dd, J=2.5, 9.0 Hz, 2H), 6.54 (dd, J=2.5, 8.5 Hz, 2H), 6.49 (d, J=2.5 Hz, 2H), 5.65 (s, 4H).

Synthesis of Compound 118: Refer to the synthesis method of Molecular Rotor 1, with a yield of 74%. ¹H-NMR (400 MHz, CDCl₃): δ=9.11 (s, 2H), 8.11 (d, J=8.5 Hz, 1H), 7.74 (m, 2H), 7.61 (m, 1H), 7.54 (m, 1H), 7.47 (m, 1H), 7.38 (d, J=9.9 Hz, 2H), 7.24 (d, J=8.5 Hz, 2H), 5.47 (s, 2H), 1.56 (s, 9H).

Synthesis of Molecular Rotor 38: Dissolve Compound 118 (69 mg, 0.16 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add ethylamine (8 mg, 0.17 mmol), the condensing agent PyBop (122 mg, 0.23 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The intermediate can be obtained after purification by column chromatography. Place the intermediate (50 mg, 0.12 mmol) and 1,3-propanesultone (30 mg, 0.24 mmol) in a 50 mL round-bottom flask, add 20 mL of acetonitrile to dissolve them. Under the protection of nitrogen, heat the reaction system in an oil bath to 100° C. for 20 hours. After the reaction is completed, cool the reaction system to room temperature, and then remove the organic solvent in the system by distillation under reduced pressure. Extract the residue with 100 mL of ethyl acetate (EA) and water, combine the organic phases, dry over anhydrous sodium sulfate, and remove the solvent by distillation under reduced pressure. The product can be obtained after purification by column chromatography (13 mg, yield 20%). ¹H-NMR (400 MHz, CD₃OD): δ=9.16 (s, 2H), 7.76 (d, J=9.5 Hz, 2H), 7.26 (d, J=2.5 Hz, 2H), 7.21 (d, J=3.5 Hz, 1H), 7.07 (d, J=3.5 Hz, 2H), 6.95 (dd, J=2.5, 9.8 Hz, 2H), 4.99 (s, 2H), 4.02 (s, 2H), 3.28 (m, 6H), 1.81 (t, J=2.5 Hz, 3H).

### Example 39

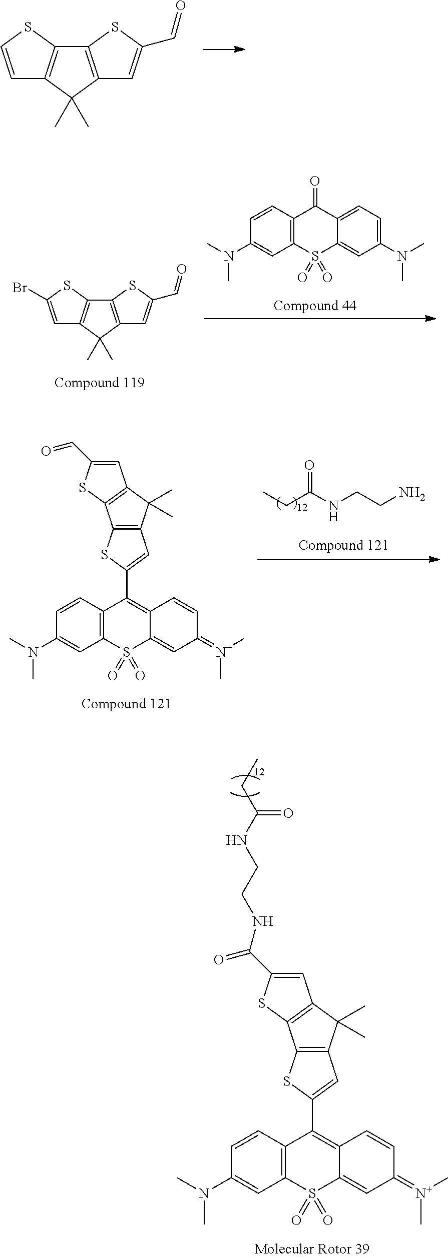

Synthesis of Compound 119: Refer to the literature Dyes and Pigments 99 (2013) 1091-1100 for synthesis, with a yield of 39%. ¹H-NMR (400 MHz, CDCl₃): δ=9.83 (s, 1H), 7.61 (s, 1H), 7.07 (d, 1H), 1.47 (s, 6H).

Synthesis of Compound 120: Refer to the synthesis method of Molecular Rotor 1, with a yield of 74%. ¹H-NMR (400 MHz, CD₃OD): δ=9.84 (s, 1H), 7.43-7.26 (m, 3H), 7.22 (d, J=7.5 Hz, 1H), 7.08 (d, J=2.8 Hz, 2H), 7.02 (s, 1H), 6.99 (s, 1H), 3.20 (s, 12H), 1.49 (s, 6H).

Synthesis of Compound 121: Refer to the literature J. Am. Chem. Soc. 2021, 143, 912-924 for synthesis, with a yield of 97%. ¹H-NMR (400 MHz, CDCl₃): δ=7.37 (s, 1H), 3.58-3.45 (m, 2H), 3.21-3.25 (m, 2H), 1.61-1.48 (m, 2H), 1.33-1.17 (m, 20H), 0.87 (t, J=7.0 Hz, 3H).

Synthesis of Molecular Rotor 39: First, oxidize the aldehyde group of Compound 120 referring to the literature Catalysis Letters volume 151, pages 36-44 (2021) to obtain the intermediate. Then dissolve the intermediate (70 mg, 0.12 mmol) in 10 mL of anhydrous DMF, add HATU (50 mg, 0.13 mmol), HOBt (9 mg, 0.06 mmol), and DIPEA (64 mg, 0.5 mmol), and stir under nitrogen protection. After stirring for 5 minutes, dissolve Compound 121 (33 mg, 0.13 mmol) and DIPEA (32 mg, 0.25 mmol) in 1 mL of anhydrous DCM, and add this solution to the above reaction system. Then stir the mixed system at room temperature for 24 hours under nitrogen protection. After the reaction is completed, remove the organic solvent in the system by distillation under reduced pressure, and then purify the crude product by column chromatography to obtain the product (56 mg, yield 44%). ¹H-NMR (400 MHz, CDCl₃): δ=8.63 (d, J=7.8 Hz, 2H), 8.25 (d, J=7.8 Hz, 1H), 7.71 (td, J=7.5, 1.0 Hz, 1H), 7.63 (td, J=7.5, 1.0 Hz, 1H), 7.56 (d, J=9.0 Hz, 2H), 6.82 (s, 1H), 6.69 (dd, J=9.0, 2.5 Hz, 2H), 3.35-3.52 (m, 8H), 2.31 (t, J=7.3 Hz, 2H), 2.18 (t, J=7.7 Hz, 2H), 1.93-2.03 (m, 2H), 1.47 (s, 6H), 1.16-1.32 (m, 23H), 0.87 (t, J=6.9 Hz, 3H).

### Example 40

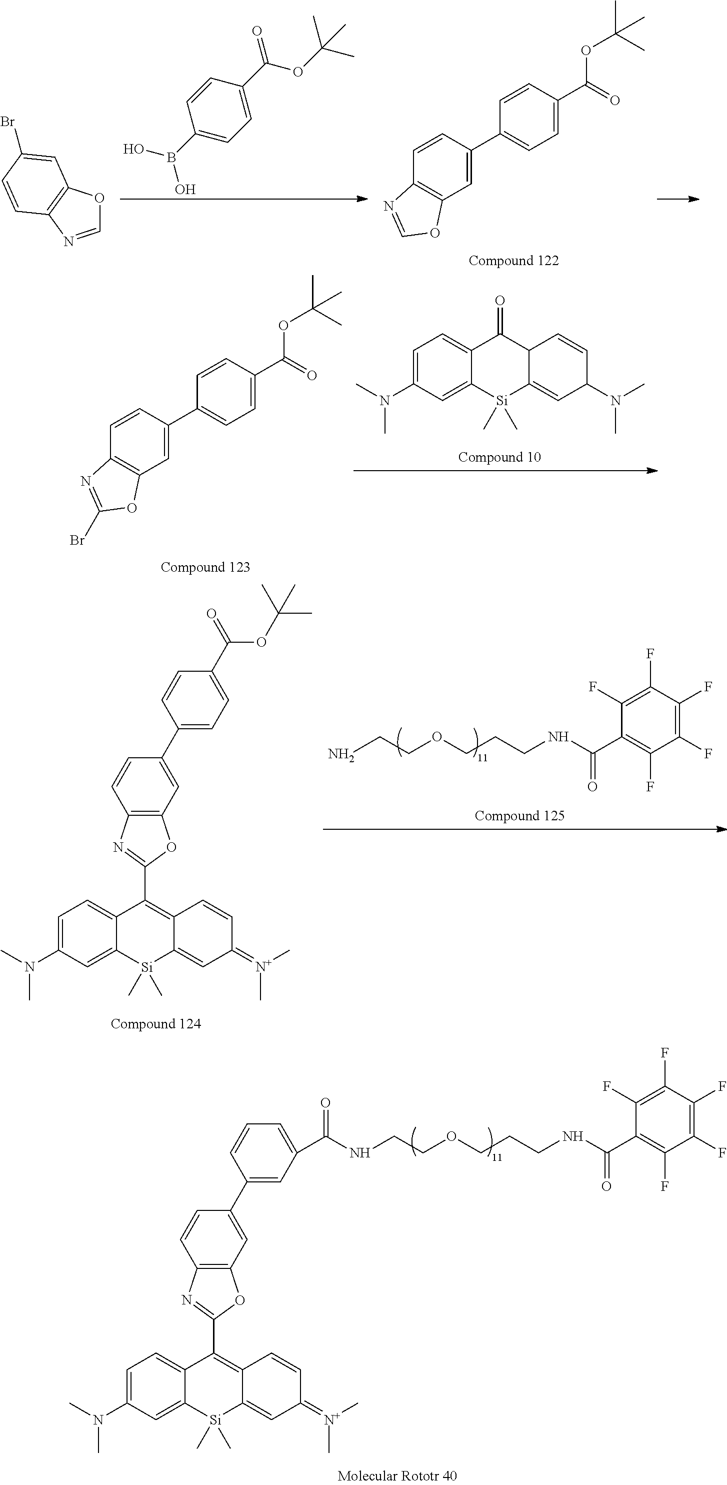

Synthesis of Compound 122: Refer to the synthesis method of Compound 40, with a yield of 92%. ¹H-NMR (400 MHz, CDCl₃): δ=8.14 (s, 1H), 8.07 (s, 1H), 7.99 (m, 1H), 7.80 (d, J=1.6 Hz, 1H), 7.46 (m, 2H), 7.38 (m, 2H), 1.39 (s, 9H).

Synthesis of Compound 123: Refer to the synthesis method of Compound 9, with a yield of 86%. ¹H-NMR (400 MHz, CDCl₃): δ=8.12 (s, 1H), 7.84 (d, J=8.4 Hz, 1H), 7.79 (d, J=1.6 Hz, 1H), 7.62 (m, 2H), 7.48 (t, J=7.2 Hz, 2H), 1.37 (s, 9H).

Synthesis of Compound 124: Refer to the synthesis method of Molecular Rotor 1, with a yield of 74%. ¹H-NMR (400 MHz, CD₃OD): δ=8.12 (s, 2H), 7.75-7.70 (m, 4H), 7.60 (s, 1H), 7.16 (s, 1H), 6.78 (t, J=7.2 Hz, 2H), 6.44 (m, 3H), 3.02 (s, 12H), 1.37 (s, 9H), 0.65 (s, 6H).

Synthesis of Compound 125: Refer to the literature J. Am. Chem. Soc. 2021, 143, 912-924 for synthesis, with a yield of 97%. ¹H-NMR (400 MHz, CDCl₃): δ=7.43 (s, 1H), 7.02 (s, 1H), 3.78 (t, J=5.3 Hz, 2H), 3.71-3.58 (m, 44H), 3.15 (sxt, J=5.4 Hz, 2H).

Synthesis of Molecular Rotor 40: Dissolve Compound 124 (93 mg, 0.15 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add Compound 125 (102 mg, 0.17 mmol), the condensing agent PyBop (117 mg, 0.23 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (162 mg, yield 61%). ¹H-NMR (400 MHz, CDCl₃): δ=8.62 (d, J=7.5 Hz, 1H), 8.25 (d, J=7.5 Hz, 1H), 7.70 (td, J=7.53, 1.2 Hz, 2H), 7.62 (td, J=7.53, 1.2 Hz, 1H), 7.57 (d, J=9.3 Hz, 1H), 7.40-7.32 (m, 3H), 6.70 (dd, J=9.0, 2.5 Hz, 1H), 6.56 (t, J=4.5 Hz, 1H), 6.49 (d, J=2.5 Hz, 1H), 6.35 (s, 1H), 3.69-3.55 (m, 44H), 3.50-3.39 (m, 8H), 3.02 (s, 12H), 2.30 (t, J=7.0 Hz, 2H), 0.67 (t, J=7.0 Hz, 6H).

### Example 41

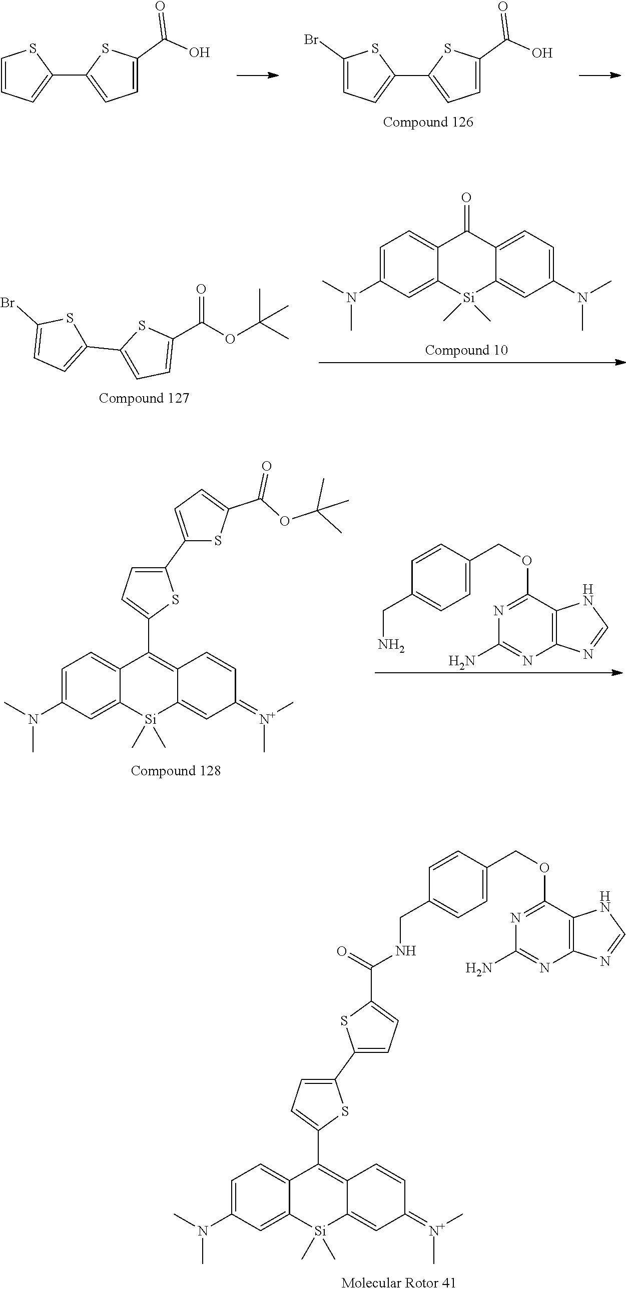

Synthesis of Compound 126: Refer to the literature J. Am. Chem. Soc. 2000, 122, 1820-1821 for synthesis, with a yield of 64%. ¹H-NMR (400 MHz, DMSO-d₆): δ=10.32 (s, 1H), 7.64 (d, J=4.0 Hz, 1H), 7.36-7.22 (m, 3H).

Synthesis of Compound 127: Refer to the synthesis method of Compound 2, with a yield of 62%. ¹H-NMR (400 MHz, CDCl₃): δ=7.87 (d, J=3.9 Hz, 1H), 7.15 (d, J=3.9 Hz, 1H), 7.12 (d, J=3.9 Hz, 1H), 7.05 (d, J=4.0 Hz, 1H), 1.45 (s, 9H).

Synthesis of Compound 128: Refer to the synthesis method of Molecular Rotor 1, with a yield of 70%. ¹H-NMR (400 MHz, CD₃OD): δ=7.68 (d, J=3.9 Hz, 2H), 7.31 (dd, J=6.2, 3.3 Hz, 2H), 7.04 (d, J=8.9 Hz, 2H), 6.83 (dd, J=8.9, 2.8 Hz, 1H), 6.77 (dd, J=9.6, 2.8 Hz, 2H), 6.40 (d, J=2.8 Hz, 1H), 3.33 (s, 12H), 0.60 (s, 3H), 0.56 (s, 3H).

Synthesis of Molecular Rotor 41: Dissolve Compound 128 (40 mg, 0.08 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add 6-[[4-(aminomethyl)phenyl]methoxy]-7H-purin-2-amine (24 mg, 0.089 mmol), the condensing agent PyBop (63 mg, 0.12 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (38 mg, yield 68%). ¹H-NMR (400 MHz, CD₃OD): δ=8.33 (s, 1H), 8.25 (m, 3H), 7.95 (d, J=7.1 Hz, 1H), 7.56 (d, J=8.0 Hz, 2H), 7.46 (d, J=8.0, 2H), 7.37 (d, J=2.6 Hz, 1H), 7.27 (d, J=7.9 Hz, 1H), 7.04 (d, J=9.6 Hz, 2H), 6.75 (dd, J=9.6, 2.6 Hz, 2H), 5.66 (s, 2H), 4.66 (s, 2H), 3.35 (s, 12H), 0.61 (s, 3H), 0.60 (s, 3H).

### Example 42

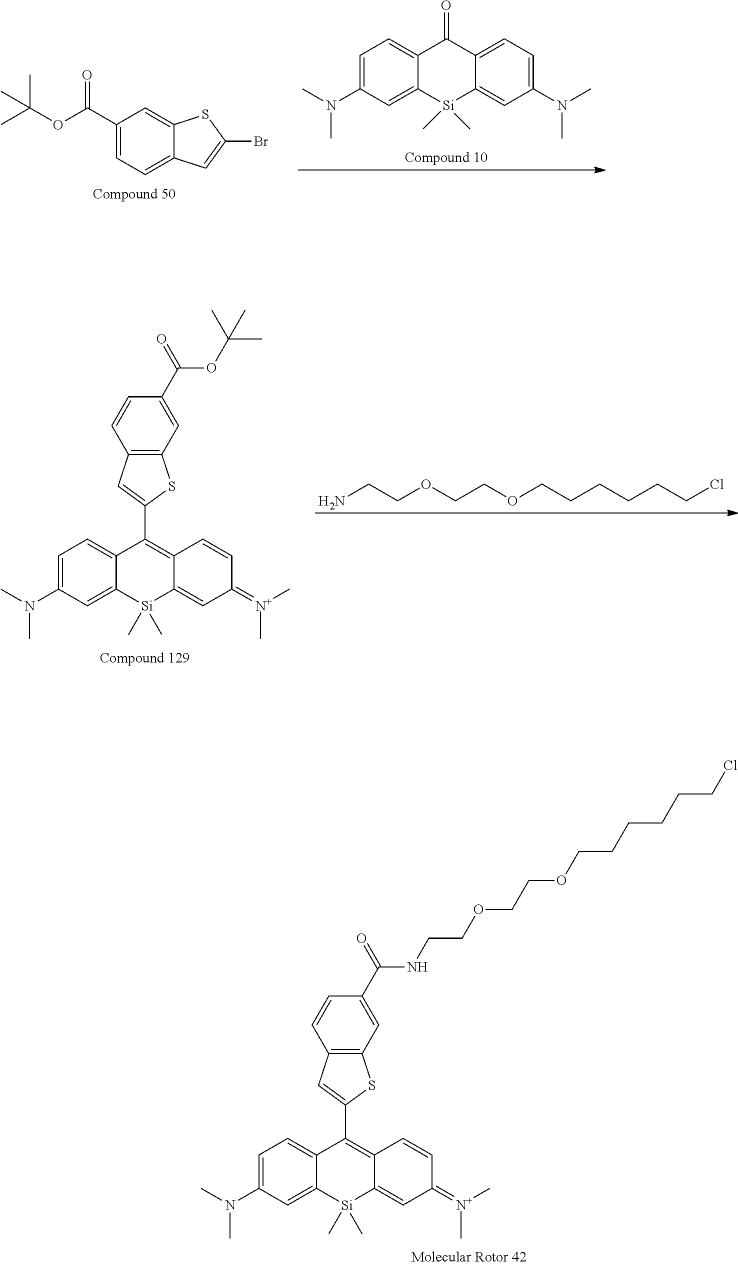

Synthesis of Compound 129: Refer to the synthesis method of Molecular Rotor 1, with a yield of 56%. ¹H-NMR (400 MHz, CD₃OD): δ=8.46 (s, 2H), 8.23 (d, J=8.0 Hz, 1H), 7.88 (t, J=7.5 Hz, 1H), 7.81 (t, J=7.5 Hz, 1H), 7.47 (d, J=7.5 Hz, 1H), 7.08 (d, J=9.5 Hz, 2H), 7.01 (d, J=9.5 Hz, 2H), 3.64 (q, J=6.5 Hz, 12H), 1.51 (s, 9H), 0.65 (s, 3H), 0.63 (s, 3H).

Synthesis of Molecular Rotor 42: Dissolve Compound 129 (40 mg, 0.08 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add 2-(2-(6-chlorohexyloxy)ethoxy)ethylamine (23 mg, 0.089 mmol), the condensing agent PyBop (63 mg, 0.12 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (18 mg, yield 34%). ¹H-NMR (400 MHz, CD₃OD): δ=8.22 (s, 1H), 7.92 (d, J=7.8 Hz, 2H), 7.37 (d, J=2.6 Hz, 2H), 7.26 (d, J=7.8 Hz, 2H), 7.05 (d, J=9.6 Hz, 2H), 6.76 (dd, J=9.6, 2.6 Hz, 2H), 4.37 (s, 2H), 3.71-3.61 (m, 8H), 3.53-3.49 (m, 4H), 3.35 (s, 12H), 1.77-1.70 (m, 2H), 1.63-1.56 (m, 2H), 1.48-1.37 (m, 2H), 0.62 (s, 3H), 0.61 (s, 3H).

### Example 43

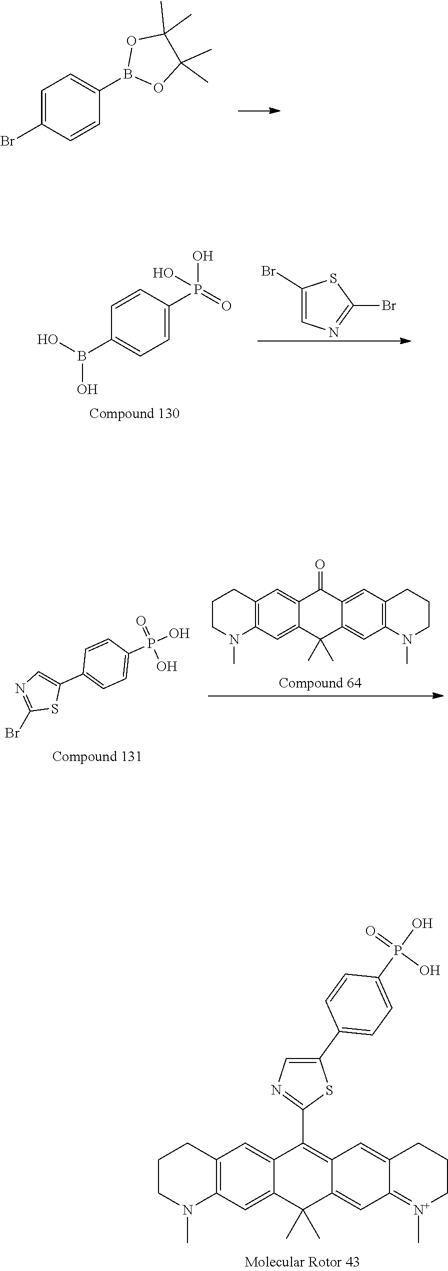

Synthesis of Compound 130: Refer to the literature Science 370, 424 (2020) for synthesis, with a yield of 80%. ¹H-NMR (400 MHz, DMSO-d₆): δ=8.46-8.30 (s, 2H), 7.85-7.82 (m, 2H), 4.62 (m, 4H).

Synthesis of Compound 131: Refer to the synthesis method of Compound 40, with a yield of 92%. ¹H-NMR (400 MHz, DMSO-d₆): δ=8.38 (s, 1H), 8.25 (s, J=15.0 Hz, 1H), 8.00-7.91 (m, 2H), 7.73-7.69 (m, 1H), 3.72 (s, 2H).

Synthesis of Molecular Rotor 43: Refer to the synthesis method of Molecular Rotor 1, with a yield of 70%. ¹H-NMR (400 MHz, CD₃OD): δ=7.40 (m, 4H), 7.22 (s, 2H), 7.08 (d, J=7.3 Hz, 1H), 6.68 (s, 2H), 4.8 (s, 2H), 3.60 (t, J=5.1 Hz, 4H), 3.33 (s, 6H), 2.48 (t, J=5.9 Hz, 4H), 1.91 (dt, J=5.9, 5.1 Hz, 4H), 0.57 (s, 3H), 0.55 (s, 3H).

### Example 44

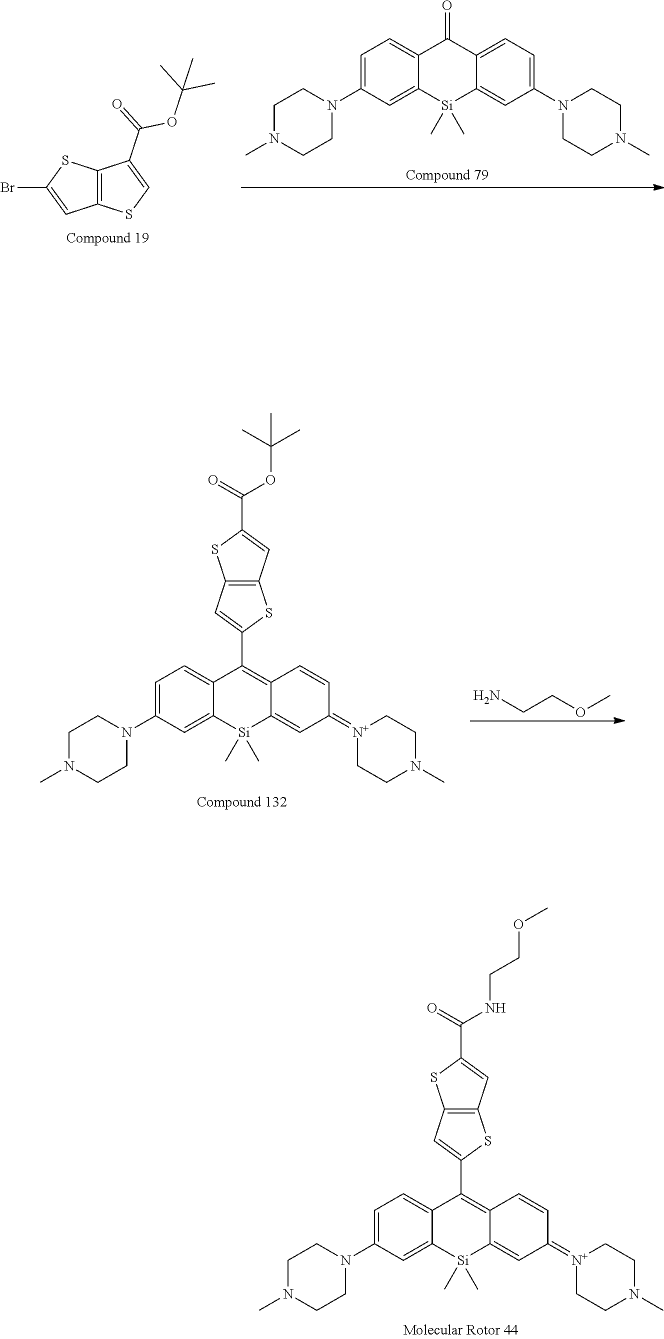

Synthesis of Compound 132: Refer to the synthesis method of Molecular Rotor 1, with a yield of 56%. ¹H-NMR (400 MHz, CD₃OD): δ=8.38 (s, 1H), 8.27 (d, J=7.8 Hz, 1H), 7.30 (d, J=8.0 Hz, 1H), 6.73 (d, J=12.3 Hz, 4H), 6.57 (d, J=8.7 Hz, 1H), 3.23 (s, 8H), 2.47 (s, 8H), 2.24 (s, 6H), 1.42 (s, 9H), 0.66 (s, 6H).

Synthesis of Molecular Rotor 44: Refer to the synthesis method of Molecular Rotor 20, with a yield of 86%. ¹H-NMR (400 MHz, CD₃OD): δ=8.41 (s, 1H), 8.30 (d, J=8.0 Hz, 1H), 7.23 (d, J=8.0 Hz, 1H), 6.89 (s, 2H), 6.82 (d, J=9.0 Hz, 2H), 6.69 (d, J=8.8 Hz, 2H), 3.72 (t, J=8.3 Hz, 2H), 3.67 (t, J=8.3 Hz, 2H), 3.63 (s, 8H), 3.54 (s, 8H), 3.20 (s, 6H), 3.04 (s, 3H), 0.66 (s, 6H).

### Example 45

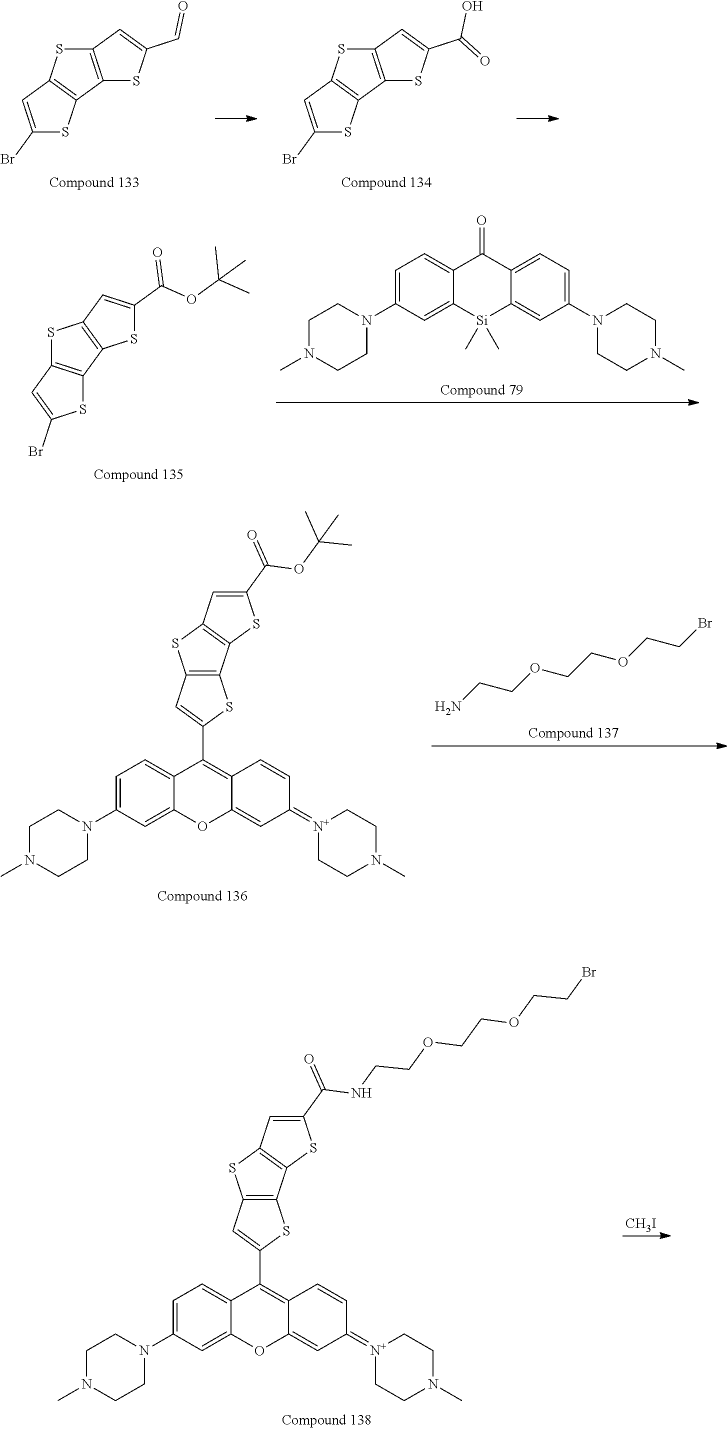

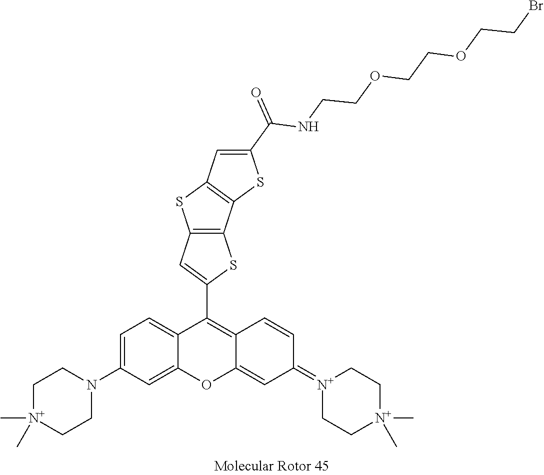 [SMILES: C[N+]1(C)CCN(c2ccc3c(-c4cc5sc6cc(C(=O)NCCOCCOCCBr)sc6c5s4)c4ccc(=[N+]5CC[N+](C)(C)CC5)cc-4oc3c2)CC1]

Synthesis of Compound 133: Refer to the literature Chemistry of Materials (2007), 19(16), 4007-4015 for synthesis, with a yield of 50%. ¹H-NMR (400 MHz, CDCl₃): δ=7.37 (d, J=5.1 Hz, 1H), 7.22 (s, 1H).

Synthesis of Compound 134: Oxidize the aldehyde group referring to the literature Catalysis Letters volume 151, pages 36-44 (2021) to obtain the product, with a yield of 83%. ¹H-NMR (400 MHz, CDCl₃): δ=7.35 (d, J=5.3 Hz, 1H), 7.24 (s, 1H).

Synthesis of Compound 135: Refer to the synthesis method of Compound 2, with a yield of 83%. ¹H-NMR (400 MHz, CDCl₃): δ=7.35 (d, J=5.3 Hz, 1H), 7.24 (s, 1H), 1.51 (s, 9H).

Synthesis of Compound 136: Refer to the synthesis method of Molecular Rotor 1, with a yield of 56%. ¹H-NMR (400 MHz, CD₃OD): δ=8.38 (s, 1H), 8.27 (d, J=7.8 Hz, 1H), 7.30 (d, J=8.0 Hz, 1H), 6.73 (d, J=12.3 Hz, 4H), 6.57 (d, J=8.7 Hz, 1H), 3.23 (s, 8H), 2.47 (s, 8H), 2.24 (s, 6H), 1.51 (s, 9H).

Synthesis of Compound 137: Refer to the literature J. Am. Chem. Soc. 2011, 133, 15280-15283 for synthesis, with a yield of 80%. ¹H-NMR (400 MHz, CDCl₃): δ=3.84-3.81 (t, 2H), 3.87-3.78 (t, 2H), 3.69-3.68 (d, 4H), 3.50-3.48 (t, 2H), 3.27 (s, 2H).

Synthesis of Compound 138: Dissolve Compound 136 (95 mg, 0.14 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add Compound 140 (33 mg, 0.16 mmol), the condensing agent PyBop (111 mg, 0.21 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (82 mg, yield 72%). ¹H-NMR (400 MHz, CD₃OD): δ=8.17 (d, 1H), 7.79 (d, 1H), 7.16 (d, 1H), 6.69 (m, 4H), 6.56 (m, 2H), 3.83, (s, 2H), 3.73 (t, 2H), 3.69 (m, 6H), 3.37 (m, 2H), 3.23 (s, 8H), 2.47 (s, 8H), 2.24 (s, 6H).

Synthesis of Molecular Rotor 45: Refer to the synthesis method of Molecular Rotor 26, with a yield of 83%. ¹H-NMR (400 MHz, CD₃OD): δ=8.41 (s, 1H), 8.30 (d, J=8.0 Hz, 1H), 7.23 (d, J=8.0 Hz, 1H), 6.89 (s, 2H), 6.82 (d, J=9.0 Hz, 2H), 6.69 (d, J=8.8 Hz, 2H), 3.73 (t, 2H), 3.69 (m, 6H), 3.63 (s, 8H), 3.54 (s, 8H), 3.37 (m, 2H), 3.20 (s, 12H).

### Example 46

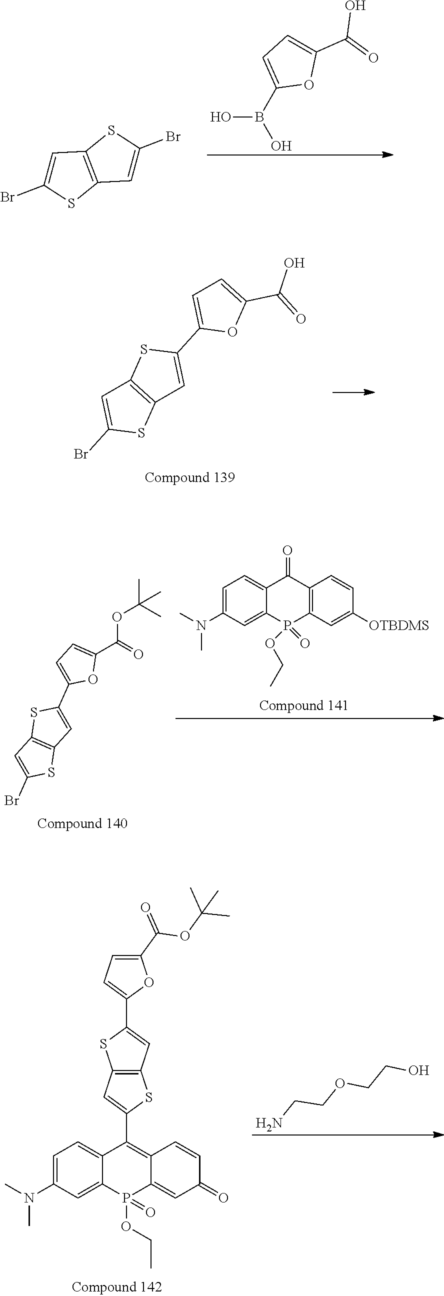

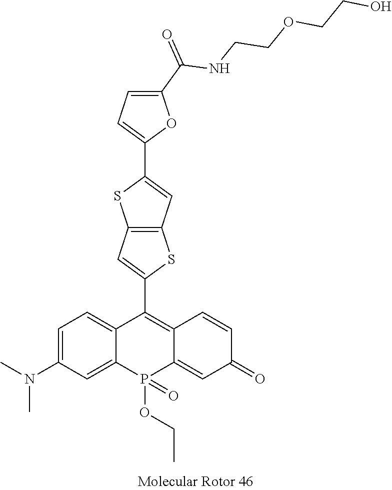 [SMILES: CCOP1(=O)C2=CC(=O)C=CC2=C(c2cc3sc(-c4ccc(C(=O)NCCOCCO)o4)cc3s2)c2ccc(N(C)C)cc21]

Synthesis of Compound 139: Refer to the synthesis method of Compound 40, with a yield of 86%. ¹H-NMR (400 MHz, CDCl₃): δ=7.35 (s, 1H), 7.25 (s, 1H), 7.12-7.01 (m, 2H).

Synthesis of Compound 140: Refer to the synthesis method of Compound 2, with a yield of 66%. ¹H-NMR (400 MHz, CDCl₃): δ=7.34 (s, 1H), 7.29 (s, 1H), 7.22-7.11 (m, 2H), 1.54 (s, 9H).

Synthesis of Compound 141: Refer to the literature Angew. Chem. Int. Ed. 2018,57,15702-15706 and Chem. Commun., 2016, 52, 12290-12293 for synthesis. ¹H-NMR (400 MHz, CDCl₃): δ=8.40 (d, J=3.7 Hz, 1H), 8.37 (d, J=3.7 Hz, 1H), 7.04 (d, J=2.9 Hz, 1H), 6.98 (dd, J=8.8 Hz, 2.2 Hz, 1H), 6.84 (dd, J=9.5 H, 2.9 Hz, 1H), 6.78 (d, J=2.9 Hz, 1H), 4.45 (s, 2H), 3.10 (s, 6H), 1.34 (q, J=6.9, 6.1 Hz, 3H).

Synthesis of Compound 142: Refer to the synthesis method of Molecular Rotor 1, with a yield of 66%. ¹H-NMR (400 MHz, CD₃OD): δ=7.58-7.40 (m, 3H), 7.32 (d, J=7.4 Hz, 1H), 7.26-7.11 (m, 1H), 7.04-6.87 (m, 3H), 6.80 (q, J=8.3 Hz, 2H), 4.10 (s, 2H), 3.21 (s, 6H), 1.56 (s, 9H), 1.34 (q, J=6.9, 6.1 Hz, 3H).

Synthesis of Molecular Rotor 46: Dissolve Compound 142 (73 mg, 0.12 mmol) in 8 mL of anhydrous DCM, then add 2 mL of trifluoroacetic acid. Stir at room temperature for 2 hours, and then remove the solvent by distillation under reduced pressure. Subsequently, dissolve the obtained solid in anhydrous DMF, add diethanolamine (14 mg, 0.13 mmol), the condensing agent PyBop (92 mg, 0.18 mmol), and 0.1 mL of triethylamine. Stir at room temperature for 1 hour. After the reaction is completed, remove the solvent by distillation under reduced pressure, extract with DCM, combine the organic phases, dry over anhydrous sodium sulfate, and remove the solvent by distillation under reduced pressure. The product can be obtained after purification by column chromatography (55 mg, yield 72%). ¹H-NMR (400 MHz, CD₃OD): δ=8.63 (dd, J=8.2, 1.1 Hz, 2H), 8.29 (dd, J=7.8, 1.0 Hz, 1H), 7.72 (td, J=8.0, 1.8 Hz, 1H), 7.65 (td, J=7.8, 1.0 Hz, 1H), 7.58 (d, J=9.3 Hz, 1H), 7.07 (t, J=4.5 Hz, 1H), 6.69 (dd, J=9.3, 2.8 Hz, 1H), 6.49 (d, J=2.8 Hz, 1H), 6.36 (s, 1H), 5.40 (s, 1H), 4.51 (t, J=9.1, 4.1, 2.6 Hz, 2H), 3.90 (m, 6H), 3.32 (d, J=3.7 Hz, 2H), 3.10 (s, 6H), 1.23 (q, J=6.9, 6.1 Hz, 3H).

### Example 47

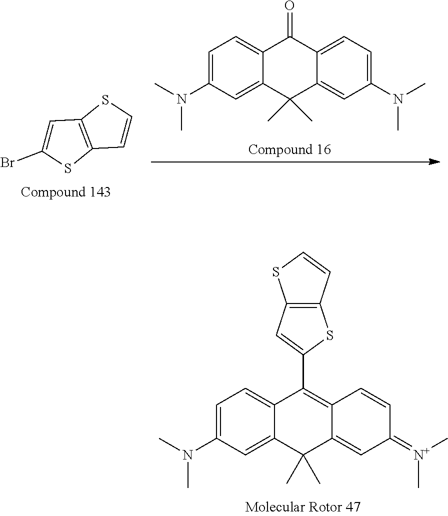

Synthesis of Molecular Rotor 47: Refer to the synthesis method of Molecular Rotor 1, with a yield of 78%. ¹H-NMR (400 MHz, CD₃OD): δ=7.47 (s, 1H), 7.31 (d, J=8.0 Hz, 1H), 7.16-7.12 (m, 2H), 7.03 (s, 1H), 6.77 (s, 1H), 6.20 (s, 1H), 5.91 (s, 1H), 3.64 (q, J=6.5 Hz, 12H), 0.65 (s, 3H), 0.63 (s, 3H).

### Example 48

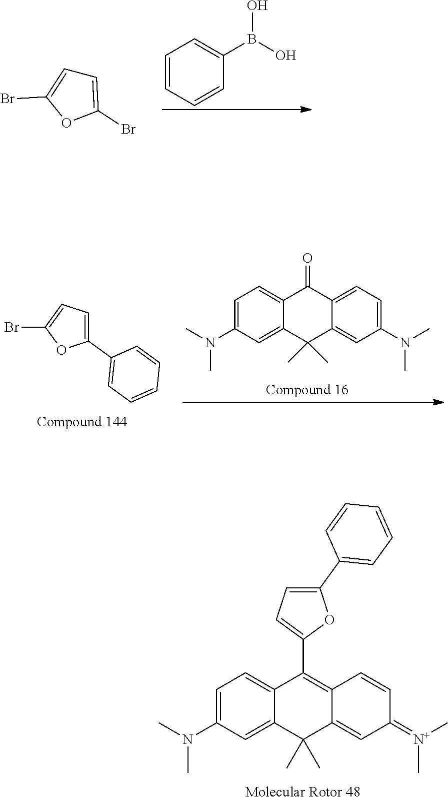

Synthesis of Compound 144: Refer to the synthesis method of Compound 40, with a yield of 86%. ¹H-NMR (400 MHz, CDCl₃): δ=8.07 (d, J=5.3 Hz, 2H), 7.60 (t, J=7.6 Hz, 3H), 7.10 (s, 1H), 6.54 (s, 1H)o Synthesis of Molecular Rotor 48: Refer to the synthesis method of Molecular Rotor 1, with a yield of 78%. ¹H-NMR (400 MHz, CD₃OD): δ=8.12 (d, J=5.2 Hz, 2H), 7.58 (t, J=6.9 Hz, 3H), 7.08 (m, 3H), 6.77 (d, J=7.6 Hz, 2H), 6.20 (d, J=6.5 Hz, 1H), 5.90 (t, J=7.8 Hz, 2H), 3.64 (q, J=6.5 Hz, 12H), 0.65 (s, 3H), 0.63 (s, 3H).

### Comparative Example 1

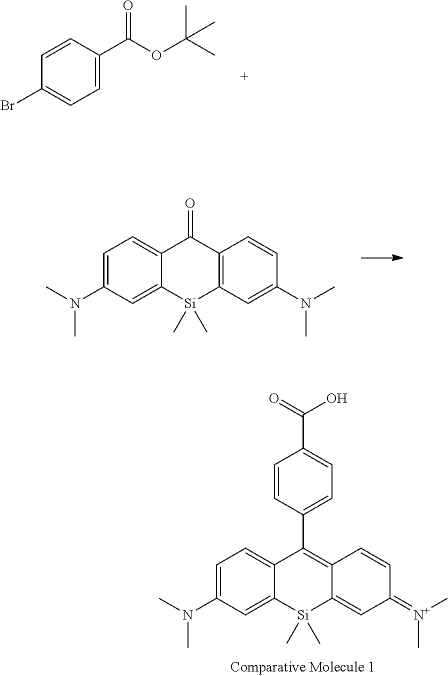

Synthesis of Comparative Molecule 1: The compound was synthesized by referring to the method reported in Analyst, 2020, 145, 7736, with a yield of 12%. ¹H-NMR (400 MHz, DMSO-d₆): δ=7.74 (d, J=8.3 Hz, 2H), 7.09 (d, J=2.9 Hz, 2H), 6.99 (d, J=7.8 Hz, 2H), 6.64 (d, J=9.3 Hz, 2H), 6.40 (dd, J=9.3, 2.9 Hz, 2H), 2.96 (s, 12H), 0.15 (s, 6H).

### Comparative Example 2

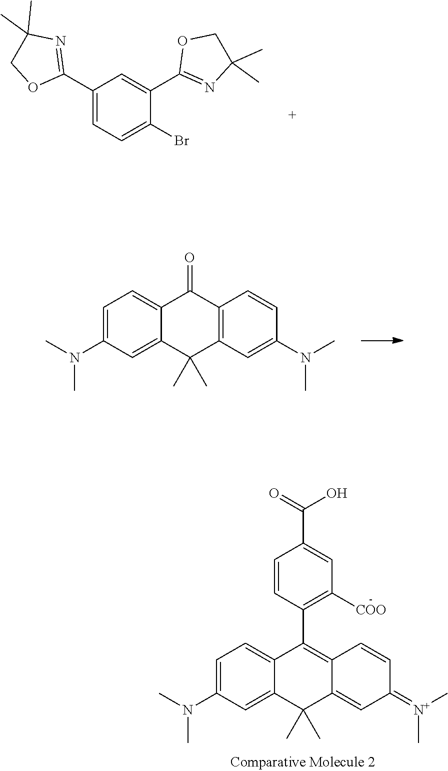

Synthesis of Comparative Molecule 2: The compound was synthesized by referring to the method reported in J. Am. Chem. Soc. 2019, 141, 7562-7571, with a yield of 47%. ¹H-NMR (400 MHz, CD₃OD): δ=8.88 (d, J=1.7 Hz, 1H), 8.40 (dd, J=7.9, 1.8 Hz, 1H), 7.47 (d, J=7.9 Hz, 1H), 7.24 (d, J=2.5 Hz, 2H), 7.01 (d, J=9.4 Hz, 2H), 6.82 (dd, J=9.4, 2.6 Hz, 2H), 3.34 (s, 3H), 1.89 (s, 3H), 1.77 (s, 3H).

### Comparative Example 3

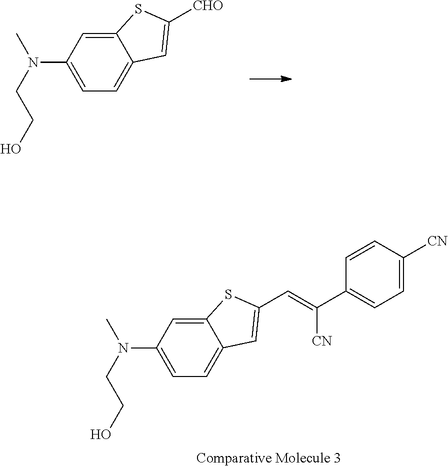 [SMILES: CN(CCO)c1ccc2cc(C=O)sc2c1 ; CN(CCO)c1ccc2cc(/C=C(\C#N)c3ccc(C#N)cc3)sc2c1]

Synthesis of Comparative Molecule 3: The compound was synthesized by referring to the method reported in Nat Biotechnol 37, 1287-1293 (2019), with a yield of 90%. ¹H-NMR (400 MHz, DMSO-d₆): δ=8.45 (s, 1H), 7.92 (d, J=8.6 Hz, 2H), 7.85 (d, J=8.3 Hz, 3H), 7.73 (dd, J=8.6, 3.9 Hz, 1H), 7.21 (d, J=1.9 Hz, 1H), 7.11 (d, J=1.9 Hz, 1H), 6.96 (dd, J=9.1, 2.3 Hz, 1H), 3.63-3.57 (m, 2H), 3.52 (t, J=5.7 Hz, 2H), 3.05 (s, 3H).

### Comparative Example 4

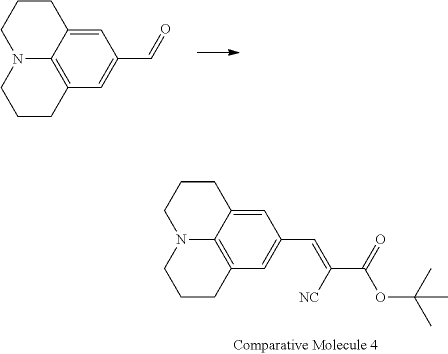 [SMILES: O=Cc1cc2c3c(c1)CCCN3CCC2 ; CC(C)(C)OC(=O)/C(C#N)=C/c1cc2c3c(c1)CCCN3CCC2]

Synthesis of Comparative Molecule 4: The compound was synthesized by referring to the method reported in Bioconjugate Chem. 2018, 29, 215-224, with a yield of 96%. ¹H NMR (400 MHz, DMSO-d₆): δ 7.85 (s, 1H), 7.48 (s, 2H), 3.31 (t, J=5.5 Hz, 4H), 2.65 (t, J=5.9 Hz, 4H), 1.90-1.79 (m, 4H), 1.5 (s, 9H).

### TEST Example 1

The fluorescent dyes (molecular rotors) prepared in Examples 1-48 were dissolved in dimethylsulfoxide, respectively, and a masterbatch of each with a concentration of 1×10⁻² M was prepared, and the respective masterbatch was added to methanol and glycerol, and mixed homogeneously, and a solution with a final concentration of each of them of 1×10⁻⁵ M was prepared, and according to the differences in the fluorescent dyes, the fluorescence emission patterns of the fluorescent dyes were detected in turn with their respective maximal excitation wavelengths under the same conditions, and the results As shown in Table 1, it indicates that the fluorescent dyes of the present invention have long excitation and emission wavelengths, and the fluorescent dyes in the near-infrared wavelength region can realize red excitation and red emission, which is convenient to be applied to in vivo fluorescence imaging. In addition, the fluorescent dyes of the present invention have high fluorescence intensity ratios in glycerol and methanol, indicating that such fluorescent dyes are responsive to viscosity changes.

TABLE 1
Excitation and emission wavelengths of each molecular
rotor in methanol and its fluorescence intensity
ratio in glycerol to methanol solution

| | Maximum | Maximum | Fluorescence |
|---|---|---|---|
| | Excitation | Emission | Intensity Ratio |
| | Wavelength | Wavelength | of Glycerol to |
| Compound Name | (nm) | (nm) | Methanol |
| Molecular Rotor 1 | 648 | 670 | 553 |
| Molecular Rotor 2 | 607 | 619 | 612 |
| Molecular Rotor 3 | 679 | 694 | 465 |
| Molecular Rotor 4 | 624 | 646 | 432 |
| Molecular Rotor 5 | 652 | 670 | 650 |
| Molecular Rotor 6 | 682 | 705 | 356 |
| Molecular Rotor 7 | 666 | 682 | 423 |
| Molecular Rotor 8 | 657 | 675 | 456 |
| Molecular Rotor 9 | 683 | 706 | 320 |
| Molecular Rotor 10 | 795 | 815 | 751 |
| Molecular Rotor 11 | 636 | 655 | 468 |
| Molecular Rotor 12 | 646 | 667 | 655 |
| Molecular Rotor 13 | 689 | 706 | 352 |
| Molecular Rotor 14 | 712 | 739 | 481 |
| Molecular Rotor 15 | 700 | 728 | 499 |
| Molecular Rotor 16 | 690 | 711 | 351 |
| Molecular Rotor 17 | 665 | 686 | 688 |
| Molecular Rotor 18 | 682 | 701 | 678 |
| Molecular Rotor 19 | 730 | 750 | 989 |
| Molecular Rotor 20 | 755 | 802 | 888 |
| Molecular Rotor 21 | 718 | 740 | 544 |
| Molecular Rotor 22 | 645 | 676 | 389 |
| Molecular Rotor 23 | 524 | 552 | 468 |
| Molecular Rotor 24 | 656 | 672 | 412 |
| Molecular Rotor 25 | 655 | 676 | 315 |
| Molecular Rotor 26 | 659 | 677 | 412 |
| Molecular Rotor 27 | 615 | 640 | 401 |
| Molecular Rotor 28 | 654 | 672 | 469 |
| Molecular Rotor 29 | 610 | 631 | 415 |
| Molecular Rotor 30 | 734 | 759 | 513 |
| Molecular Rotor 31 | 776 | 801 | 472 |
| Molecular Rotor 32 | 620 | 641 | 212 |
| Molecular Rotor 33 | 559 | 582 | 431 |
| Molecular Rotor 34 | 591 | 615 | 612 |
| Molecular Rotor 35 | 634 | 656 | 376 |
| Molecular Rotor 36 | 642 | 662 | 501 |
| Molecular Rotor 37 | 681 | 705 | 379 |
| Molecular Rotor 38 | 593 | 625 | 423 |
| Molecular Rotor 39 | 736 | 764 | 421 |
| Molecular Rotor 40 | 689 | 711 | 562 |
| Molecular Rotor 41 | 684 | 702 | 679 |
| Molecular Rotor 42 | 679 | 705 | 598 |
| Molecular Rotor 43 | 741 | 762 | 416 |
| Molecular Rotor 44 | 652 | 683 | 231 |
| Molecular Rotor 45 | 564 | 592 | 212 |
| Molecular Rotor 46 | 597 | 628 | 502 |
| Molecular Rotor 47 | 675 | 698 | 402 |
| Molecular Rotor 48 | 681 | 707 | 463 |

### Test Example 2

Molecular rotors 2, 9, 12, 19, and 40 were added to methanol and glycerol, respectively, and prepared into solutions with a final concentration of 1×10⁻⁵ M. The fluorescence spectra in glycerol and methanol were tested according to their respective optimal excitation wavelengths for excitation, and the absorption values at excitation wavelengths were tested by UV spectrometry, and the relative fluorescence quantum yields were obtained by the method of relative fluorescence quantum yields. quantum yields in methanol and glycerol. In addition, their respective maximum absorption values in methanol were tested by UV spectroscopy, and their respective molar extinction coefficients were calculated by Lambert's Beer's law. The experimental data are summarized in Table 2, which shows that the fluorescent dyes of the present invention have high molar extinction coefficients, low fluorescence quantum yields in the free state (methanol), and high fluorescence quantum yields in environments with high viscosities such as glycerol.

TABLE 2
The Photophysical Properties of Representative
Molecular Rotors in Methanol and Glycerol

| Compound | Eₓ/ | Eₘ/ | ε | QY | QY |
|---|---|---|---|---|---|
| Name | nm | nm | (M⁻¹ cm⁻¹) | (Methanol) | (Glycerin) |
| Molecular | 607 | 619 | 98700 | 0.0012 | 0.62 |
| Rotor 2 | | | | | |
| Molecular | 683 | 706 | 128000 | 0.0005 | 0.39 |
| Rotor 9 | | | | | |
| Molecular | 646 | 667 | 130000 | 0.0046 | 0.55 |
| Rotor 12 | | | | | |
| Molecular | 730 | 750 | 100000 | 0.0001 | 0.15 |
| Rotor 19 | | | | | |
| Molecular | 689 | 711 | 100350 | 0.0024 | 0.50 |
| Rotor 40 | | | | | |

### Test Example 3

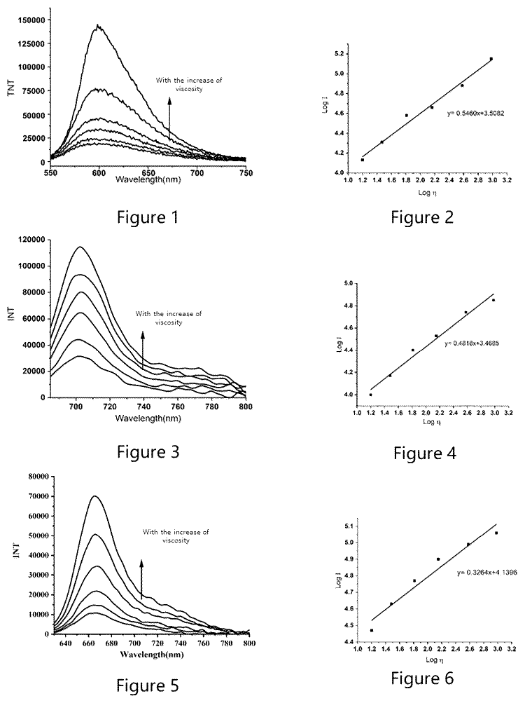

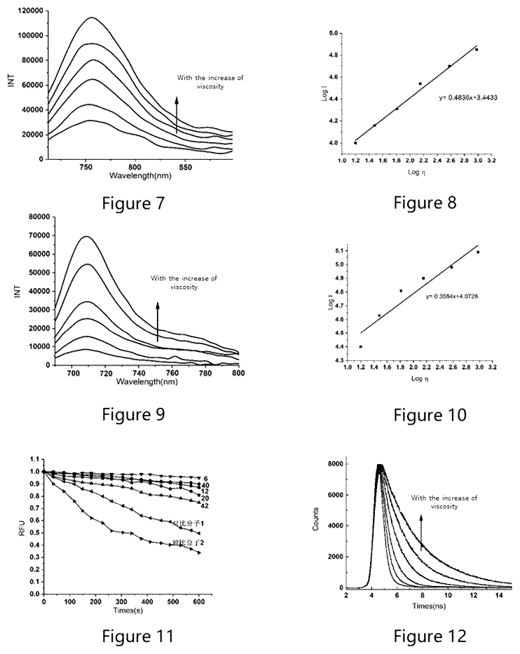

The molecular rotors 2, 9, 12, 19 and 40 were added to the ethylene glycol-glycerol mixed solution and formulated into a solution with a final concentration of 1×10⁻⁵ M. The fluorescence emission spectra under different viscosity conditions were excited according to their respective optimal excitation wavelengths, as shown in FIGS. 1, 3, 5, 7, and 9, and the fluorescence intensities of the same concentration of molecular rotors under different viscosity conditions gradually increased, indicating that the fluorescence intensity of the molecular rotor fluorescence increased with the increase of the ambient viscosity. The fluorescence intensity of the molecular rotor increases with the ambient viscosity, and the relationship between the logarithm of the fluorescence intensity and the logarithm of the solvent viscosity is in accordance with the Huffman equation with a good linear relationship, as shown in FIGS. 2, 4, 6, 8 and 10, which proves that the molecular rotor is sensitive to the viscosity and can be used for viscosity testing of unknown samples.

### Test Example 4

We tested the photostability of Molecular Rotors 6, 12, 20, 40, 42 and Comparative Molecules 1, 2 by using the fluorescence decay method. We placed them on a confocal laser scanning microscope respectively, and continuously irradiated the samples with a laser at the corresponding excitation wavelength and recorded the changes in fluorescence intensity. The test results are shown in FIG. 11. The fluorescence intensity of the molecular rotors of the present invention decreases slightly as the illumination time increases, while the fluorescence intensities of Comparative Molecules 1, 2 which are considered to have good photostability decrease significantly. This indicates that the molecular rotors of the present invention have excellent photostability and are more suitable for long-term dynamic tracking and fluorescence imaging of biological substances.

### Test Example 5

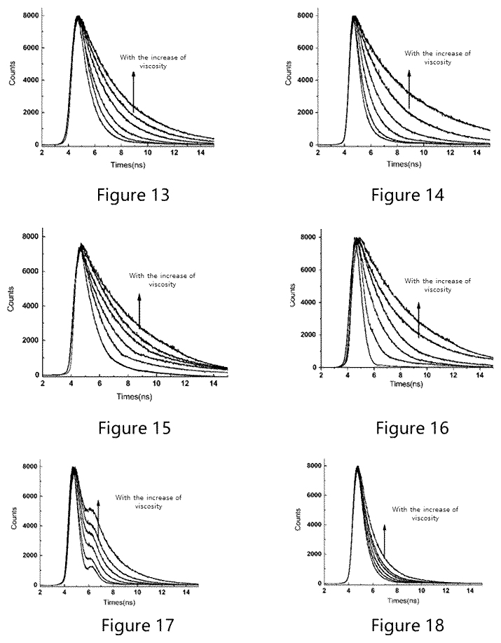

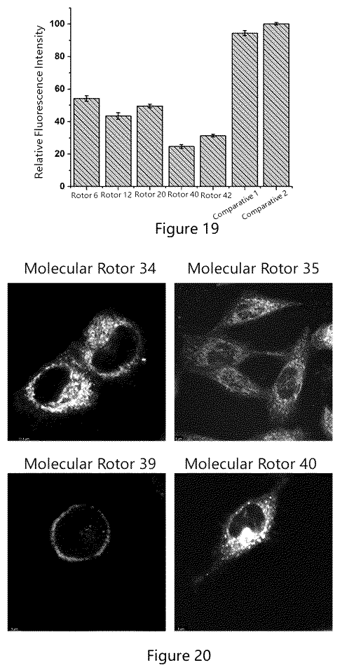

Molecular Rotors 6, 12, 19, 20, 41 and Comparative Molecules 3, 4 were respectively added to an ethylene glycol-glycerol mixed solution to prepare solutions with a final concentration of 1×10⁻⁵ M. They were excited at their respective optimal excitation wavelengths, and the fluorescence lifetimes under different viscosity conditions were tested. The experimental results of Molecular Rotors 6, 12, 19, 20, 41 are shown in FIG. 12, FIG. 13, FIG. 14, FIG. 15 and FIG. 16. For molecular rotors at the same concentration, as the viscosity of the microenvironment they are in increases, the fluorescence lifetimes gradually increase. This indicates that the information of the microenvironment they are in can be reflected by the changes in fluorescence lifetimes, so as to realize the fluorescence lifetime imaging of biological molecules. In addition, the molecular rotors of the present invention have relatively long fluorescence lifetimes, and accurate test results can also be obtained in solutions with low viscosity (ethylene glycol). However, the traditional molecular rotor (Comparative Molecule 3) has a short fluorescence lifetime, and its fluorescence lifetime is only about 0.1 ns in a solution with high viscosity such as glycerol (FIG. 17), so it is difficult to accurately detect in a low-viscosity system and is not suitable for the fluorescence lifetime imaging of biological substances. Moreover, although the fluorescence lifetime of Comparative Molecule 4 reaches the detectable range (FIG. 18), its change is not obvious in environments with different viscosities, so it is also not suitable for the fluorescence lifetime imaging of biological substances. The above shows that the molecular rotor fluorescent dyes of the present invention have relatively long fluorescence lifetimes and the changes are obvious with the change of viscosity, and are more suitable for the fluorescence lifetime imaging of biological substances.

### Test Example 6

Molecular Rotors 6, 12, 20, 40, 42 and Comparative Molecules 1, 2 were added to the cell culture medium to prepare solutions with a final concentration of 1×10⁻⁶ M, which were then used to culture Hela cells. After incubation for 2 hours, each compound was excited at its maximum excitation wavelength, and their fluorescence intensities were detected. All samples were normalized with the strongest fluorescence of all groups set as 100, as shown in FIG. 19. The results indicate that, compared with the dyes having similar structures (Comparative Molecules 1 and 2), the molecular rotors of the present invention have lower background fluorescence.

### Test Example 7

Molecular Rotors 34, 35, 39 and 40 can specifically localize to subcellular organelles (such as lysosomes, mitochondria, cell membranes, endoplasmic reticulum, etc.) and perform specific fluorescence imaging of these subcellular organelles, as shown in FIG. 20. The dyes emit bright fluorescence only at the locations of the subcellular organelles, while the non-specific fluorescence at other locations in the cells and the background fluorescence in the culture medium are very weak. This indicates that the dyes of the present invention have good cell membrane permeability. After targeting the subcellular organelles, their fluorescence can be activated by the microenvironment of the organelles, and they can be used for specific fluorescence imaging of subcellular organelles.

### Test Example 8

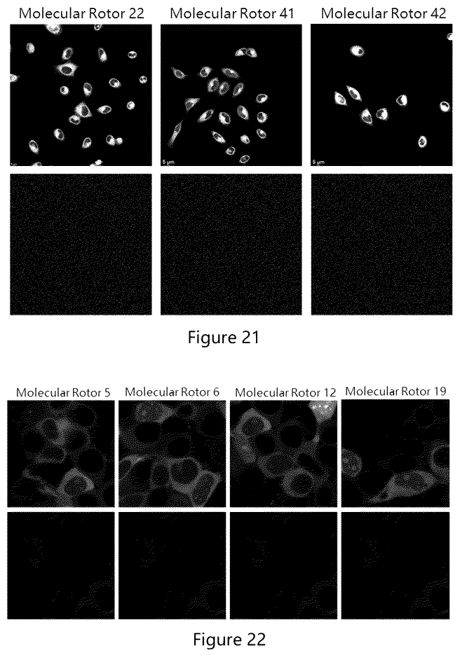

Molecular Rotors 22, 41 and 42 can specifically bind to the protein tags Halo-Tag and SNAP-Tag. The dyes basically do not emit fluorescence before binding, and the fluorescence is significantly activated after binding. As shown in FIG. 21, the cells expressing the tagged proteins emit bright fluorescence, while the cells without protein expression do not have fluorescence. This indicates that the dyes of the present invention can be used for the labeling of proteins in living cells.

### Test Example 9

Molecular Rotors 5, 6, 12 and 19 can be used for the screening of aptamers and can specifically bind to the obtained RNA aptamers. The background of the dyes is very low before binding, and the fluorescence is significantly activated after binding. As shown in FIG. 22, the dyes can also bind to the RNA aptamers in living cells and emit bright fluorescence, with basically no non-specific fluorescence activation. This indicates that the dyes of the present invention have very low background fluorescence and low non-specific fluorescence in cells, and can be used for the labeling of low-abundance biological macromolecules such as nucleic acids. The sequence of one of the RNA a tamers is:

AGACGCAACUGAAUGAAACAUUCCCACCUUAGAGUGACUGUGCCUG
CUUCGGCAGGACAGGUGCAUGUCAACCGCUGUCACUCCGUAACUAGUCG
CGUCAC.

### Test Example 10

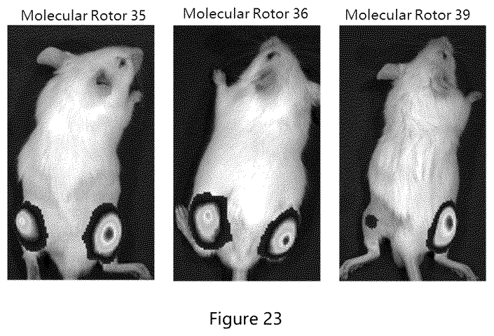

Molecular Rotors 35, 36 and 39 can achieve fluorescence imaging of tumors in vivo. Subcutaneous sarcomas were implanted in the legs of mice, and the dyes were injected intravenously. The experimental results are shown in FIG. 23. The tumor sites on the legs of the mice showed obvious fluorescence signals. This indicates that the fluorescent dyes of the present invention can specifically accumulate at the tumor sites and exhibit strong fluorescence signals, and can be used for in vivo fluorescence imaging of tumors.

1. A fluorescent dye having a structure as shown in formula (I), 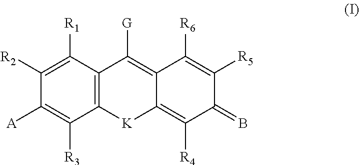
wherein:
A is —N(X₁)(X₂) or HO—, B is ═N⁺(X₃)(X₄) or ═O, or X₁, X₂ is linked to R₂ and/or R₃, or X₃, X₄ is linked to R₄ and/or R₅ as a heterocyclic group;
X₁, X₂, X₃, and X₄ are each independently selected from the group consisting of hydrogen, alkyl, modified alkyl, aryl, heteroaryl, unsaturated or saturated heterocyclic groups, or optionally, X₁ and X₂, or X₃ and X₄ may be connected to form a saturated or unsaturated, unsubstituted or R¹⁻¹-substituted heterocyclic or heteroaromatic ring; Additionally, X₁, X₂ together with R₂ and/or R₃, or X₃, X₄ together with R₄ and/or R₅, may optionally be connected to form a saturated or unsaturated, unsubstituted or R¹⁻¹-substituted heterocyclic ring;
and/or R₁, R₂, R₃, R₄, R₅, and R₆ may optionally be independently selected from the group consisting of hydrogen, halogen atoms, nitro groups, or alkyl groups;
when K is selected from S, Se, Te, Te═O, unsubstituted or singly or doubly R¹⁻¹-substituted carbon, unsubstituted or singly or doubly R¹⁻¹-substituted Ge, 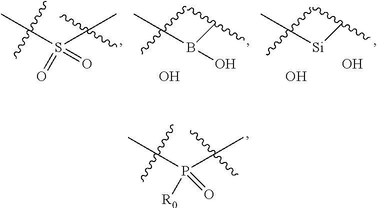
(wherein R₀ is independently and optionally selected from 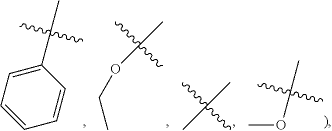
G is selected from the group consisting of formula (I-1-1), formula (I-1-2), formula (I-1-3), or formula (I-1-4);
when K is selected from O or unsubstituted or singly or doubly R¹⁻¹-substituted silicon, and A is not —N(X₁)(X₂), G is selected from the group consisting of formula (I-1-1), formula (I-1-2), formula (I-1-3), or formula (I-1-4);
when K is selected from O or unsubstituted or singly or doubly R¹⁻¹-substituted silicon, A is —N(X₁)(X₂), and B is ═N⁺(X₃)(X₄), G is selected from the group consisting of formula (I-1-2) or formula (I-1-3); 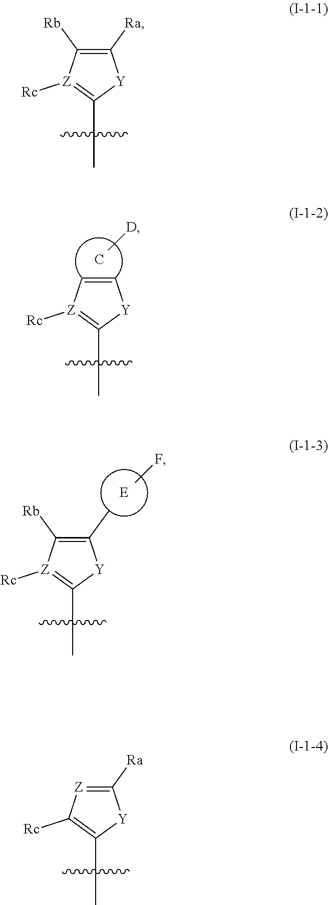
wherein:
each R¹⁻¹ is independently selected from the group consisting of halogen atoms or alkyl groups;
each Y is independently selected from the group consisting of O, S, NH, Se, or Te;
each Z is independently selected from the group consisting of C or N;
each Rₐ, R_{b}, and R_{c} is independently selected from the group consisting of hydrogen, sulfonic acid groups, haloacyl groups, cyano groups, cyanate esters, isothiocyanates, carboxyl groups, ester groups, amide groups, nitro groups, halogen atoms, alkyl groups, haloalkyl groups, or modified alkyl groups;
the C ring of formula (I-1-2), the E ring of formula (I-1-3) are each independently selected from at least one of an aromatic ring or an aromatic heterocycle;
each hydrogen atom in formula (I-1-2) and in formula (I-1-3) is optionally independently substituted with an atom selected from a halogen atom, or an alkyl group;
D in formula (I-1-2), F in formula (I-1-3) are each independently selected from hydrogen, cyano, amino, isothiocyano, carboxy, sulfone, phosphite, ester, amide, nitro, halogen atom, alkyl, aryl, heteroaryl, or modified alkyl;
wherein:
the term “alkyl” refers to a C₁-C₁₀ straight-chain or branched-chain alkyl group; preferably, it is a C₁-C₇ straight-chain or branched-chain alkyl group; more preferably, it is a C₁-C₅ straight-chain or branched-chain alkyl group; most preferably, it is selected from the group consisting of methyl, ethyl, n-propyl, isopropyl, n-butyl, isobutyl, tert-butyl, sec-butyl, n-pentyl, 1-methylbutyl, 2-methylbutyl, 3-methylbutyl, isopentyl, 1-ethylpropyl, neopentyl, n-hexyl, 1-methylpentyl, 2-methylpentyl, 3-methylpentyl, isohexyl, 1,1-dimethylbutyl, 2,2-dimethylbutyl, 3,3-dimethylbutyl, 1,2-dimethylbutyl, 1,3-dimethylbutyl, 2,3-dimethylbutyl, 2-ethylbutyl, n-heptyl, 2-methylhexyl, 3-methylhexyl, 2,2-dimethylpentyl, 3,3-dimethylpentyl, 2,3-dimethylpentyl, 2,4-dimethylpentyl, 3-ethylpentyl, or 2,2,3-trimethylbutyl;
the term “modified alkyl” refers to an alkyl group in which any carbon atom is substituted by at least one group selected from the group consisting of halogen atoms, —O—, —OH, —CO—, —NO₂, —CN, —S—, —SO₂—, —(S═O)—, optionally halogen-substituted aryl groups, amino groups, secondary amino groups, tertiary amino groups, heterocyclic groups, and saturated or unsaturated monocyclic or heterocyclic groups; The modified alkyl group contains 1 to 30 carbon atoms (preferably 1 to 20 carbon atoms, more preferably up to 10 carbon atoms), and any carbon-carbon single bond therein may optionally and independently be replaced by a carbon-carbon double bond or a carbon-carbon triple bond;
the substitution of the aforementioned carbon atom means that the carbon atom or the carbon atom together with the hydrogen atoms bonded to it is replaced by the corresponding group;
the term “heterocyclic group” refers to a 4-10 membered monocyclic or polycyclic heterocyclic group containing at least one heteroatom selected from N, O, S, or Si in the ring;
the term “aryl” or “aromatic ring” refers to a 5-10 membered monocyclic or fused bicyclic aromatic group;
the term “heteroaryl” or “aromatic heterocycle” refers to a 5-10 membered monocyclic or fused bicyclic heteroaromatic group containing at least one heteroatom selected from N, O, S, or Si in the ring;
the halogen atoms in the so-called “halogen atom”, “haloalkyl group”, “haloacyl group” and “aryl group substituted by halogen atoms” are each independently selected from F, Cl, Br and I;
the term “ester group” refers to the —(C═O)OR¹⁻² group;
the term “amide group” refers to the —CONR¹⁻² group;
the term “sulfonyl group” refers to the —SO₂R¹⁻² group;
the term “sulfoxide group” refers to the —SOR¹⁻² group;
the term “secondary amino group” refers to the —NHR¹⁻² group;
the term “tertiary amino group” refers to the —NR¹⁻²R¹⁻³ group;
each of R¹⁻² and R¹⁻³ is independently selected from H, alkyl, modified alkyl, or a halogen atom.

2. The fluorescent dye according to claim 1, wherein,
X₁ and X₂ may optionally be connected to R₂ and/or R₃ to form a saturated or unsaturated heterocyclic group, which is selected from any of the following groups: 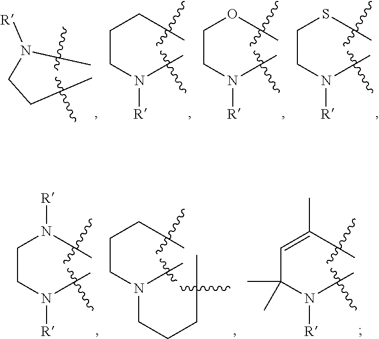
X₃ and X₄ may optionally be connected to R₄ and/or R₅ to form a saturated or unsaturated heterocyclic group, which is selected from any of the following groups: 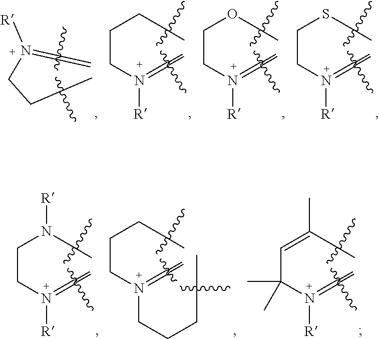
preferably, the heterocyclic group formed by X₁, X₂, and R₂ and/or R₃, or by X₃, X₄, and R₄ and/or R₅, has a symmetrical structure;
preferably, each R′ is independently selected from hydrogen or an alkyl group;
preferably, X₁ and X₂ may optionally interconnect to form a saturated or unsaturated heterocyclic group selected from any of the groups represented by the following formulas, wherein the groups may optionally be substituted with a halogen atom; 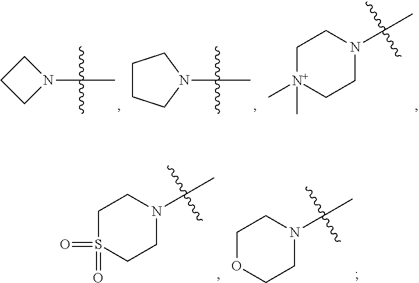
preferably, X₃ and X₄ may optionally interconnect to form a saturated or unsaturated heterocyclic group selected from any of the groups represented by the following formulas, wherein the groups may optionally be substituted with a halogen atom; 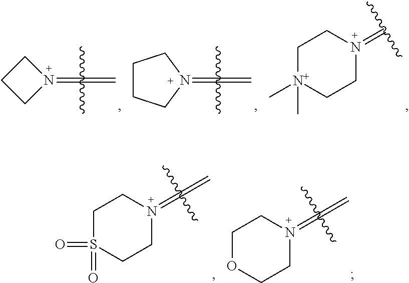
preferably, the heterocyclic groups formed by X₁ and X₂, and by X₃ and X₄, have a symmetrical structure;
preferably, K is selected from oxygen, sulfur, selenium, tellurium, or the following groups: 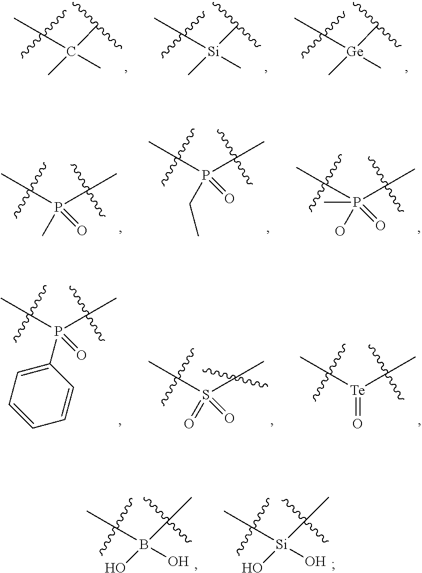
preferably, formula (I-1-1) and (I-1-4) are selected from any of the following groups: 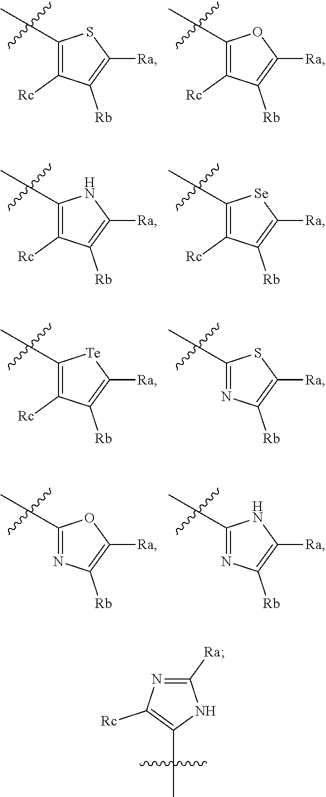
preferably, formula (I-1-2) may be selected from any of the following groups: 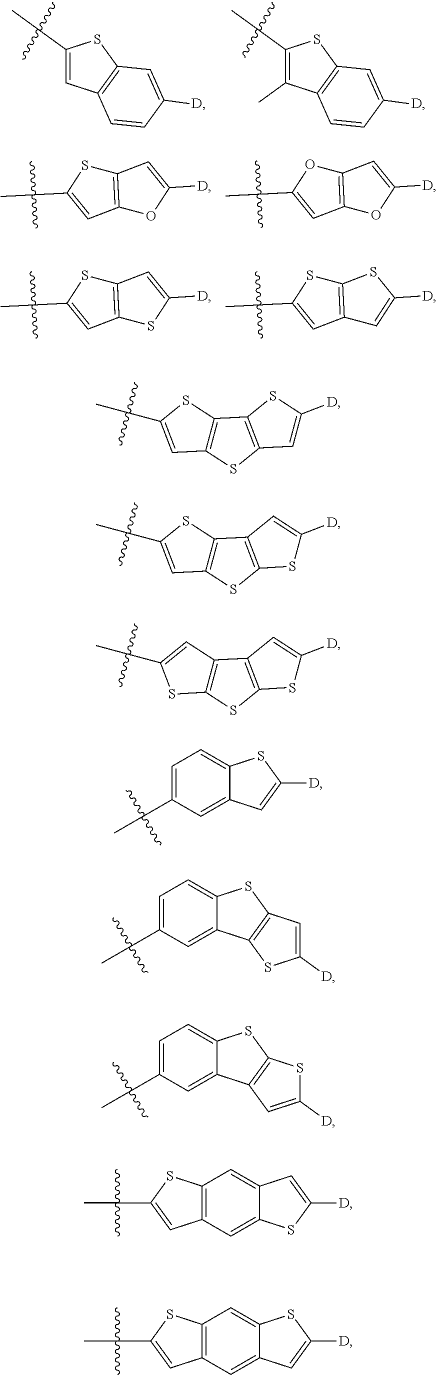 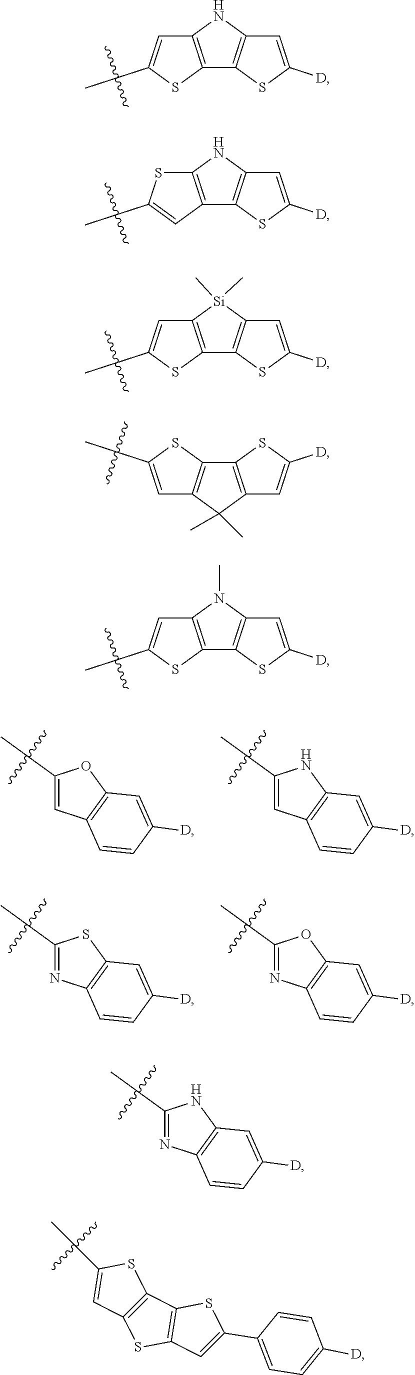 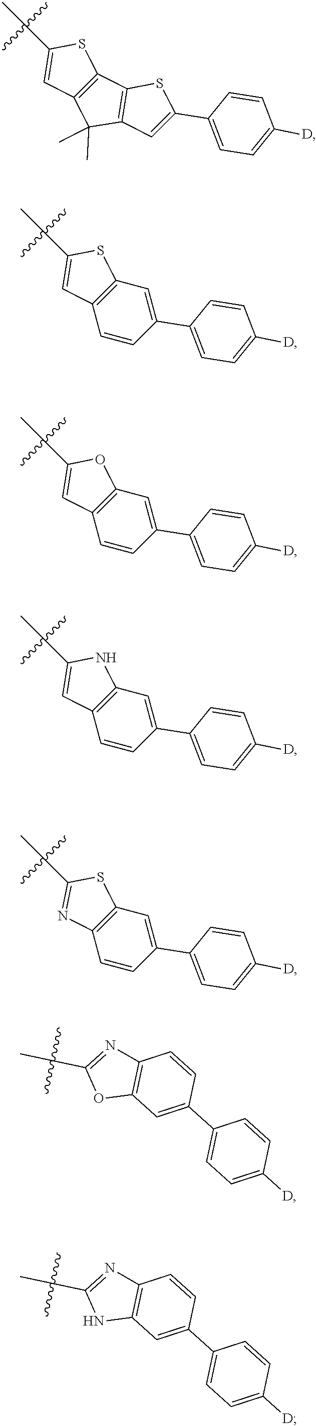
preferably, formula (I-1-3) may be selected from any of the following groups:

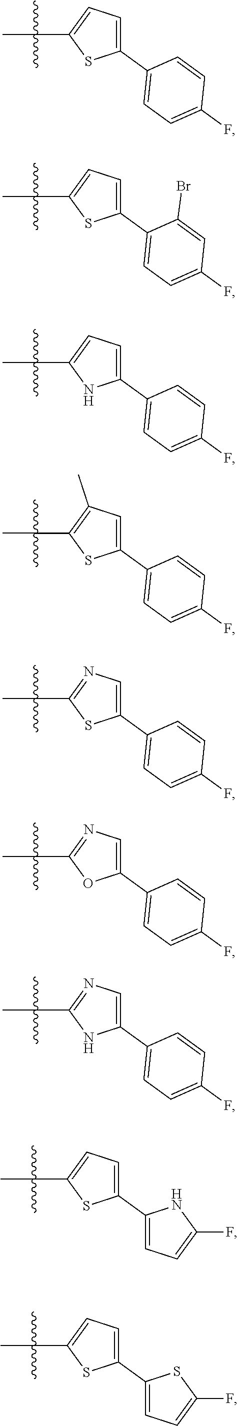

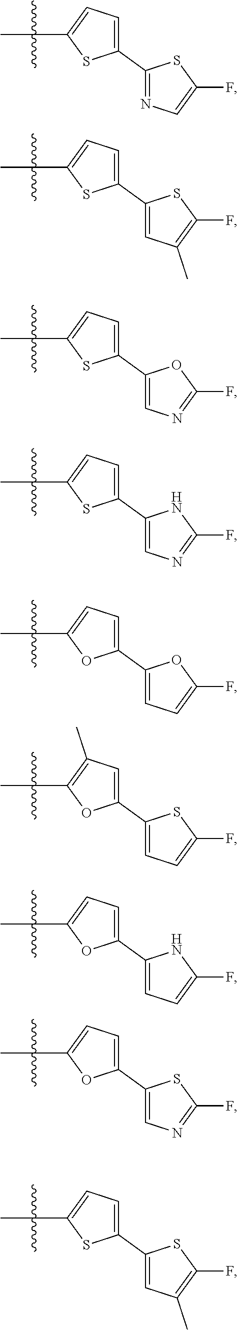

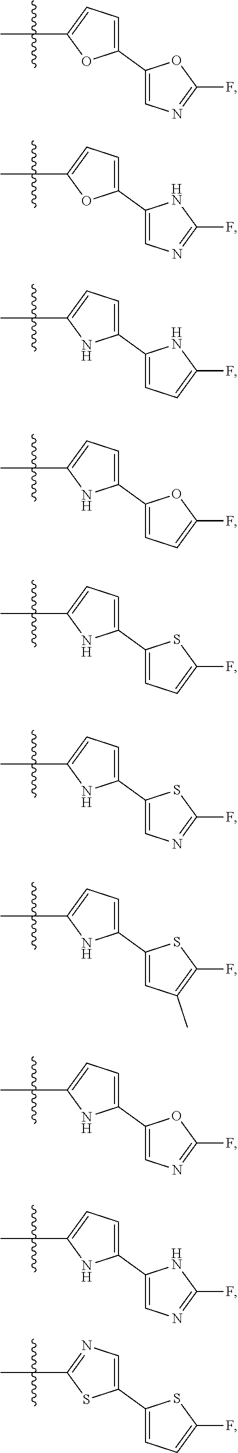

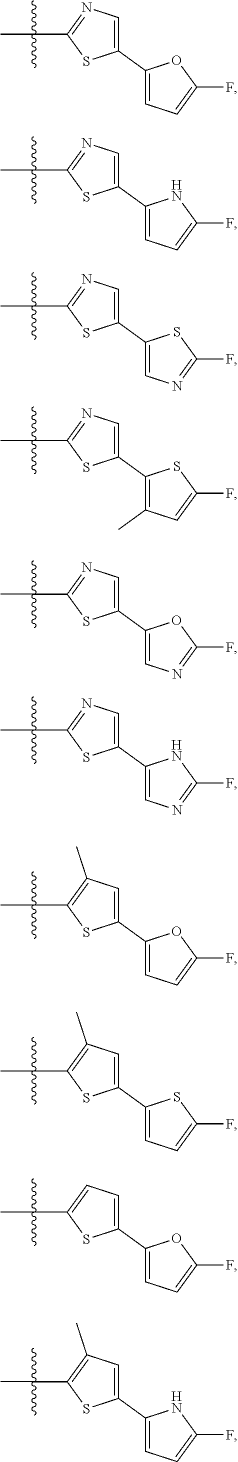

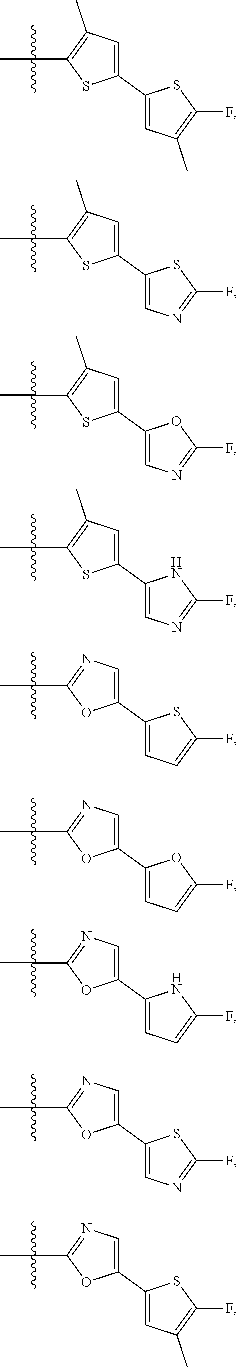

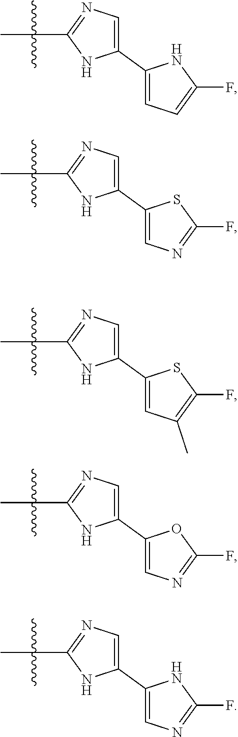 [SMILES: *c1ncc(-c2ccc(F)[nH]2)[nH]1 ; *c1ncc(-c2cnc(F)s2)[nH]1 ; *c1ncc(-c2cc(C)c(F)s2)[nH]1 ; *c1ncc(-c2cnc(F)o2)[nH]1 ; *c1ncc(-c2cnc(F)[nH]2)[nH]1]

3. The fluorescent dye according to claim 2, wherein the fluorescent dye is selected from the following compounds:

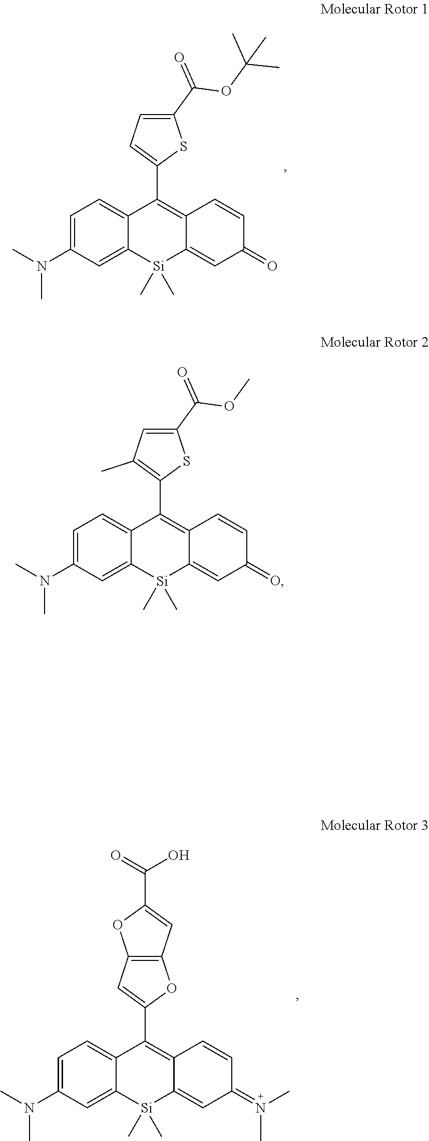

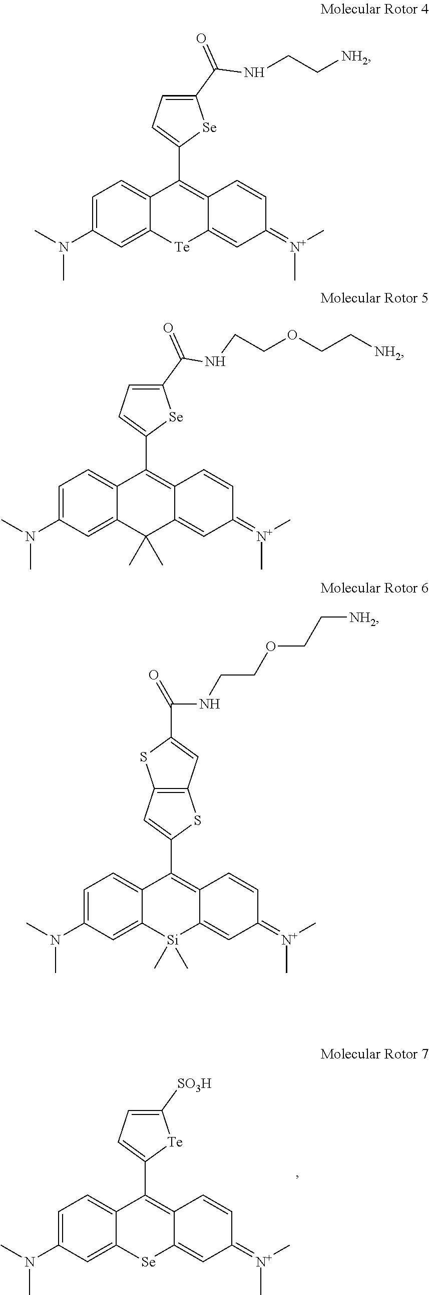

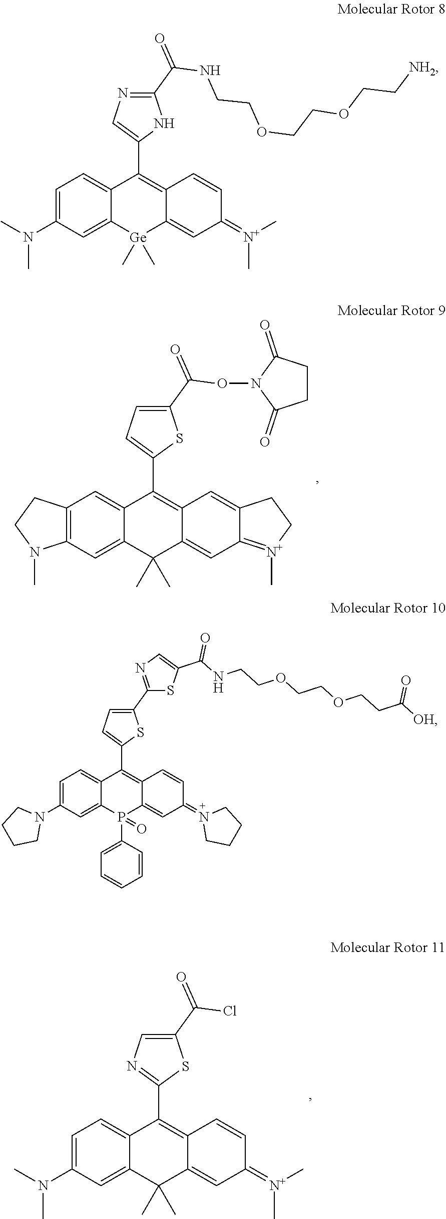

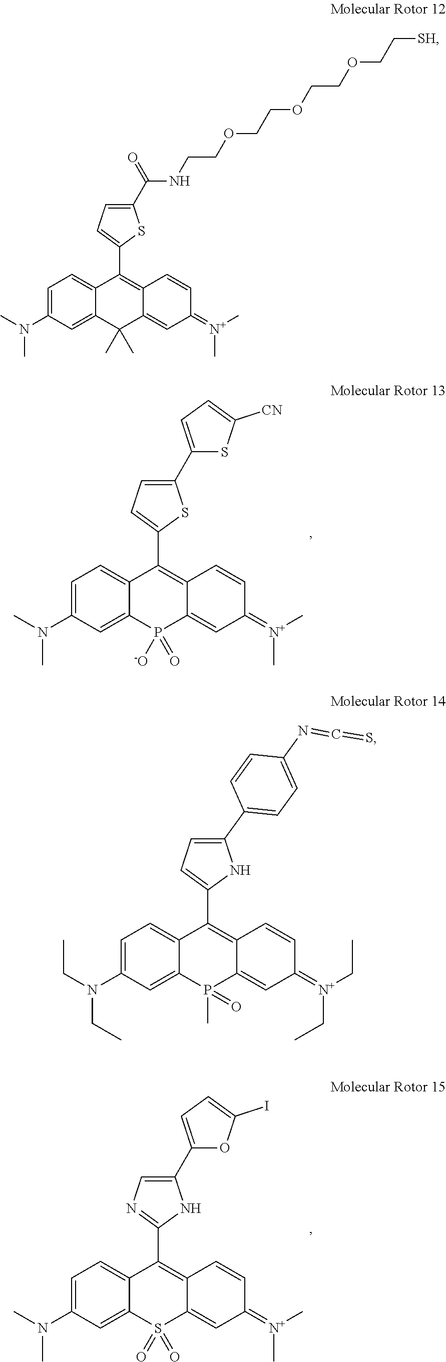

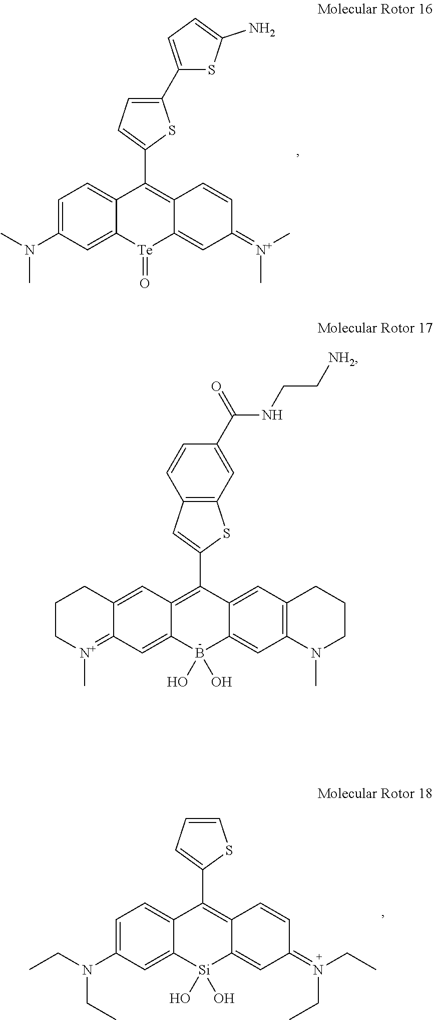

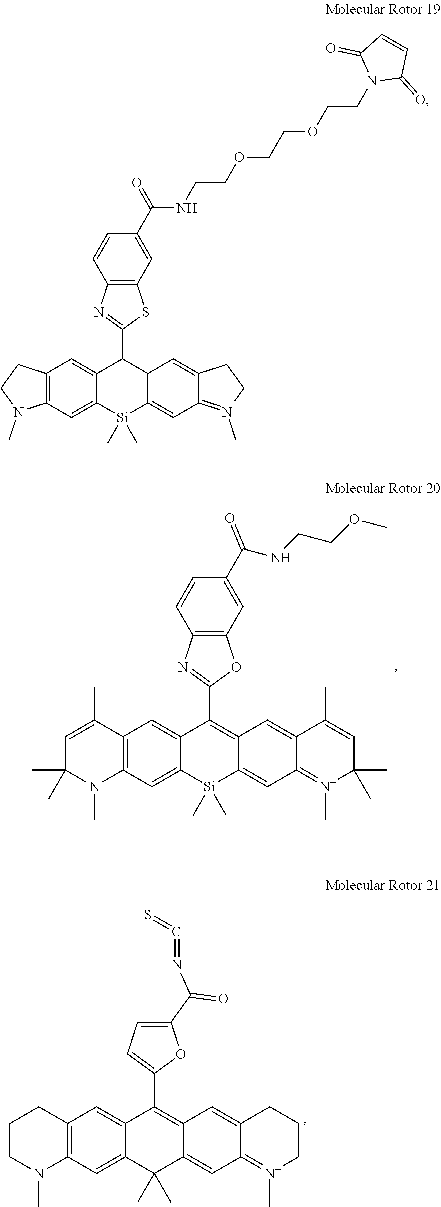

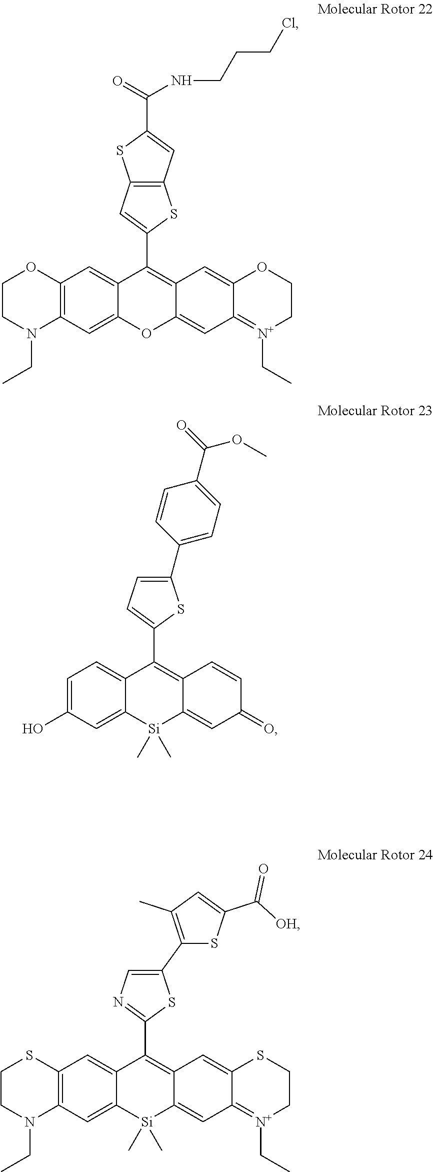 [SMILES: CCN1CCOc2cc3c(cc21)Oc1cc2c(cc1=C3c1cc3sc(C(=O)NCCCCl)cc3s1)OCC[N+]=2CC ; COC(=O)c1ccc(-c2ccc(C3=C4C=CC(=O)C=C4[Si](C)(C)c4cc(O)ccc43)s2)cc1 ; CCN1CCSc2cc3c(cc21)[Si](C)(C)c1cc2c(cc1=C3c1ncc(-c3sc(C(=O)O)cc3C)s1)SCC[N+]=2CC]

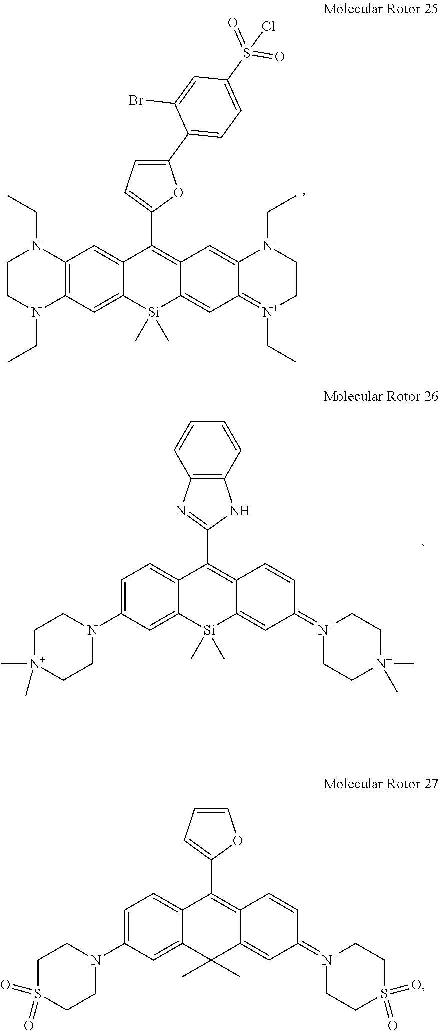

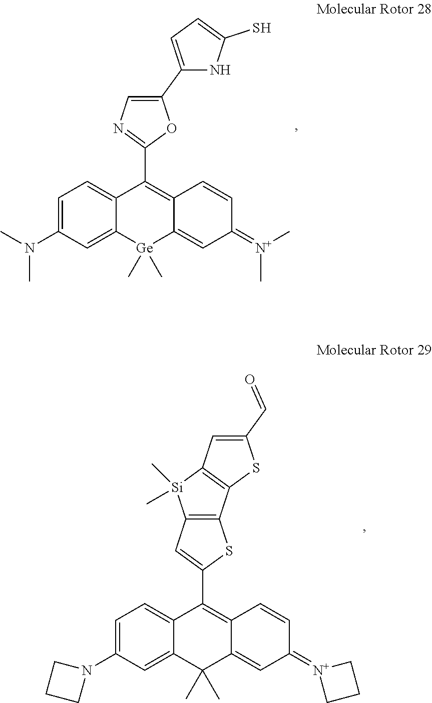

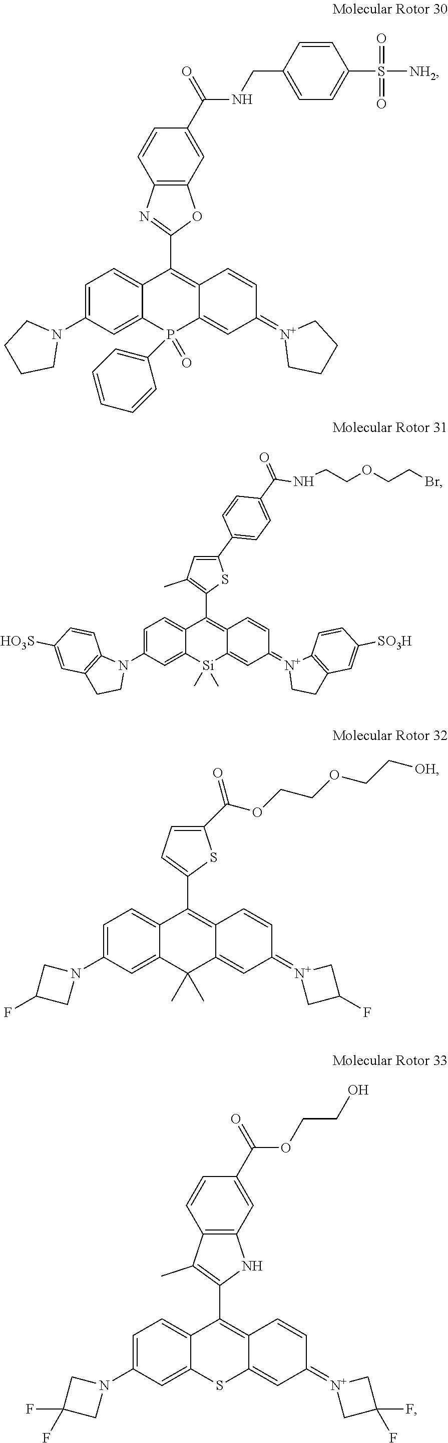 [SMILES: NS(=O)(=O)c1ccc(CNC(=O)c2ccc3nc(C4=C5C=CC(=[N+]6CCCC6)C=C5P(=O)(c5ccccc5)c5cc(N6CCCC6)ccc54)oc3c2)cc1 ; Cc1cc(-c2ccc(C(=O)NCCOCCBr)cc2)sc1C1=C2C=C/C(=[N+]3/CCc4cc(S(=O)(=O)O)ccc43)C=C2[Si](C)(C)c2cc(N3CCc4cc(S(=O)(=O)O)ccc43)ccc21 ; CC1(C)C2=CC(=[N+]3CC(F)C3)C=CC2=C(c2ccc(C(=O)OCCOCCO)s2)c2ccc(N3CC(F)C3)cc21 ; Cc1c(-c2c3ccc(=[N+]4CC(F)(F)C4)cc-3sc3cc(N4CC(F)(F)C4)ccc23)[nH]c2cc(C(=O)OCCO)ccc12]

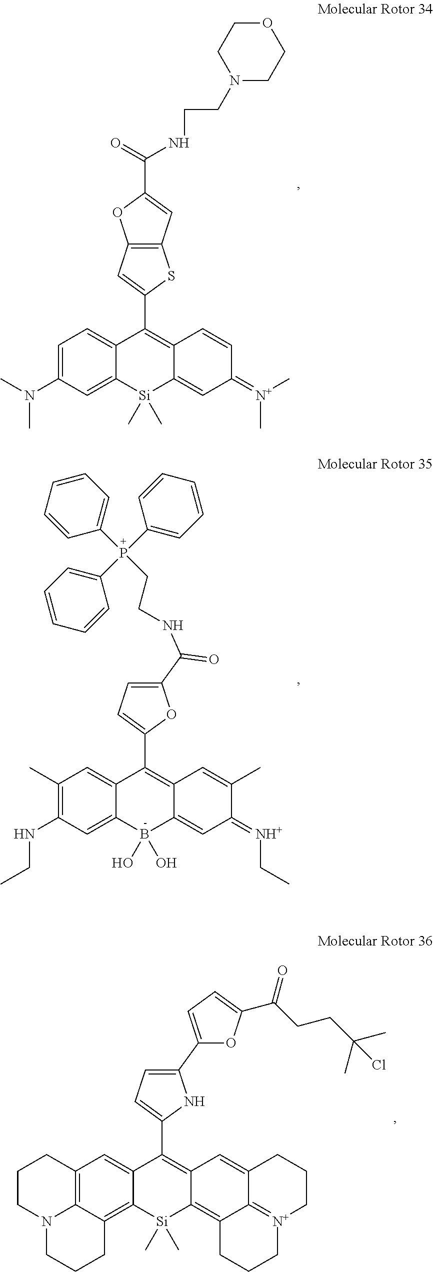

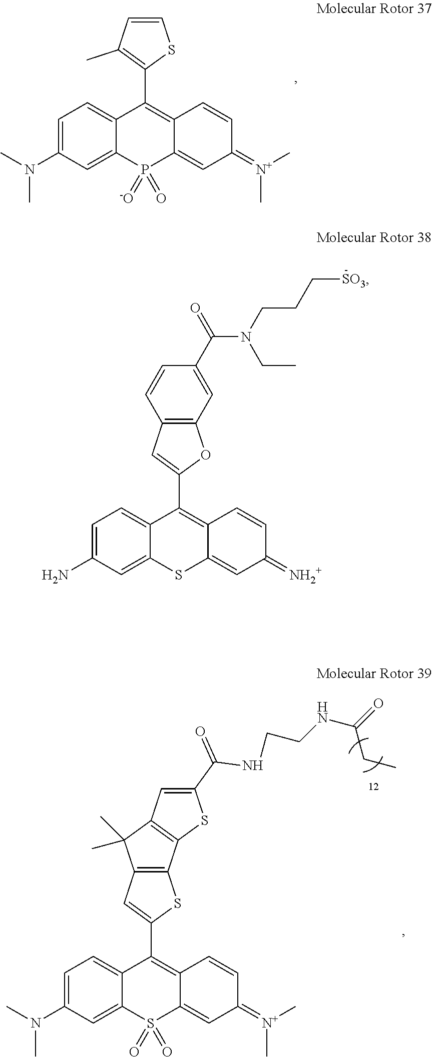

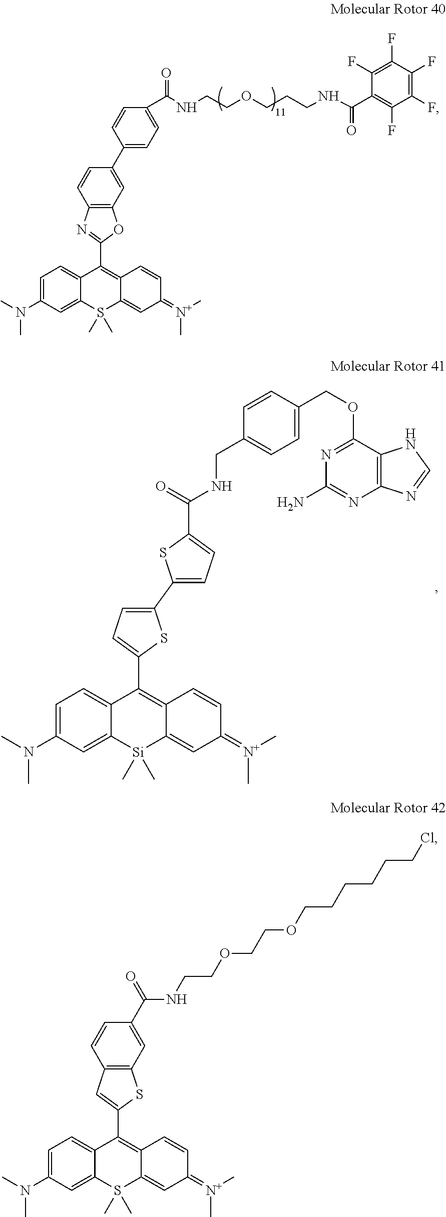

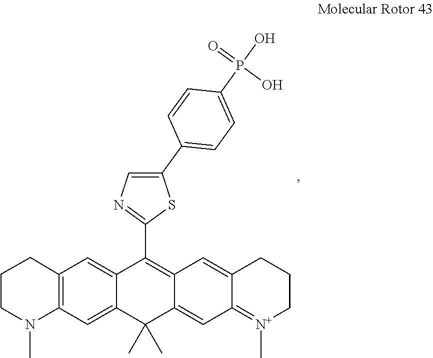 [SMILES: CN1CCCc2cc3c(cc21)C(C)(C)c1cc2c(cc1=C3c1ncc(-c3ccc(P(=O)(O)O)cc3)s1)CCC[N+]=2C]

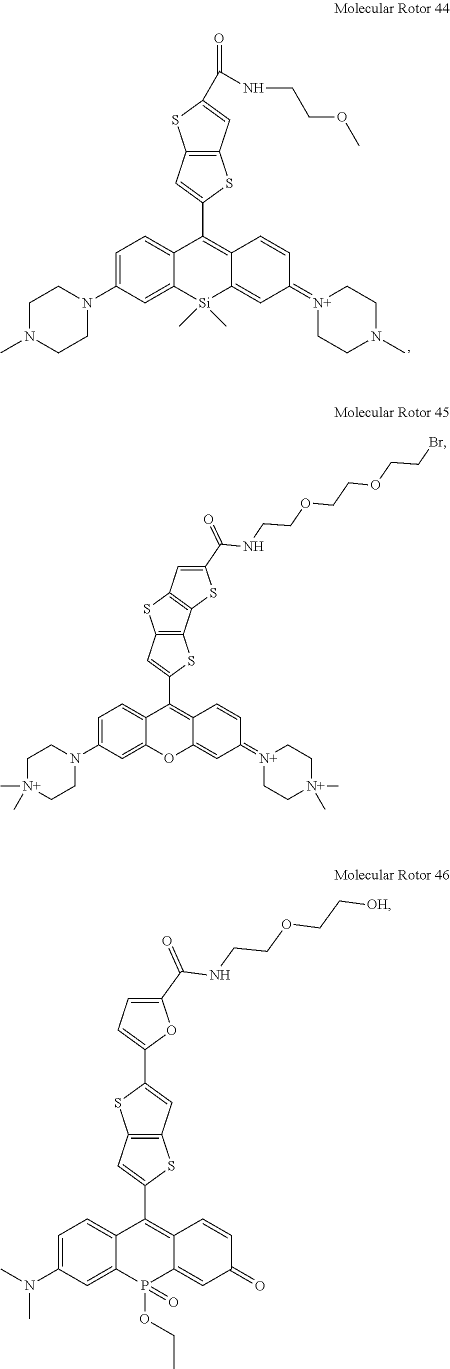

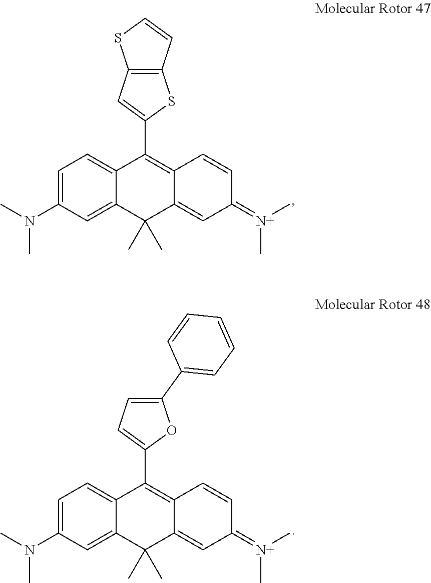

4. A method for preparing the fluorescent dye according to claim 3, further comprising a step of reacting a brominated aromatic ring compound represented by formula (II-1-1), (II-1-2), (II-1-3), or (II-1-4) with a ketone represented by formula (II-2) to obtain the fluorescent dye represented by formula (I):

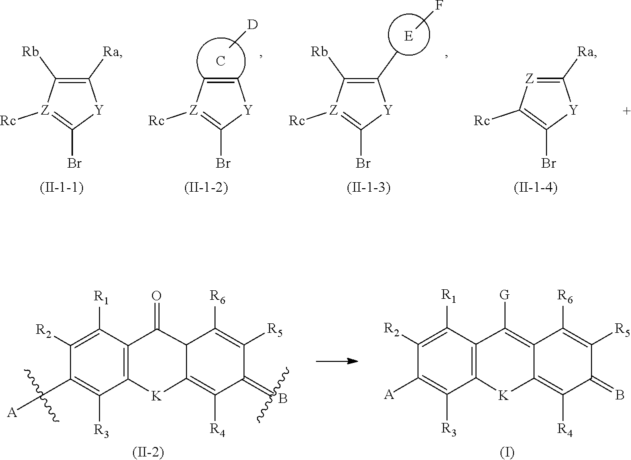

5. A fluorescence-activated biospecific labeling method, comprising the following steps:
contacting the fluorescent dye according to claim 3 with a biological substance selected from the group consisting of proteins, RNA, and DNA, wherein the fluorescent dye forms a covalent or non-covalent bond with the biological substance, thereby specifically activating the fluorescence of the dye molecule to achieve specific fluorescent labeling of the biological substance;
wherein, the reaction medium for the labeling reaction is selected from the group consisting of a pure analyte solution (pure protein, pure DNA, and pure RNA), a cell lysate, and an in situ medium in which the analyte resides; and wherein, the in situ medium is selected from the group consisting of an intracellular medium, an organelle medium, a living tissue medium, blood, and bodily fluids.

6. A probe kit comprising:
the fluorescent dye according to claim 3; and
a biocompatible medium selected from the group consisting of dimethyl sulfoxide, a buffer solution, a phosphate buffer solution, and physiological saline.

7. A method of preparing a pharmaceutical composition comprising:
selecting one of the fluorescent dyes according to claim 3; and
combining the fluorescent dye with a carrier compound to form the pharmaceutical composition, wherein the pharmaceutical composition is suitable for administration to animals or humans, and further wherein the carrier compound is selected from the group consisting of
water, saline, buffers, and preservatives.

8. A method for preparing a pharmaceutical formulation for use in disease detection comprising:
selecting at least one of the fluorescent dyes according to claim 3; and
incorporating the selected fluorescent dye into the pharmaceutical formulation.

9. The method according to claim 8, further comprising:
obtaining a biological sample from a subject;
adding the pharmaceutical formulation to the biological sample to obtain a test sample,
wherein the selected fluorescent dye is configured to bind to a target nucleic sequence associated with a disease; and
examining the test sample for the presence of the selected fluorescent dye,
wherein the presence of the selected fluorescent dye corresponds to the presence of the target nucleic sequence, and further wherein the target nucleic sequence is selected from the group consisting of antibodies, nucleic acid aptamers, and disease biomarkers.

10. The method according to claim 9 wherein:
the step of examining the test sample further comprises an operation of
in vivo fluorescence imaging of the subject;
fluorescence imaging of an ex vivo tissue sample from the subject; or fluorescence lifetime imaging of the subject, wherein the subject is human or another mammal.

11. The method according to claim 10 wherein:
the step of examining the test sample is conducted with an optical imaging system selected from the group consisting of imaging devices, optical fibers, endoscopes, and laparoscopes configured for fluorescence imaging.
